(19) United States
(12) Patent Application Publication
(10) Pub. No.: US 2005/0036951 A1
(43) Pub. Date: Feb. 17, 2005

(54) Methods of treating lung diseases

(75) Inventor: Daniel R. Henderson, Del Mar, CA (US)

Correspondence Address: FOLEY & LARDNER, P.O. BOX 80278, SAN DIEGO, CA, 92138-0278, US

(73) Assignee: Arizeke Pharmaceuticals, Inc.

(21) Appl. No.: 10/754,485

(22) Filed: Jan. 9, 2004

Related U.S. Application Data
(60) Provisional application No. 60/439,373, filed on Jan. 9, 2003.
(60) Provisional application No. 60/480,047, filed on Jun. 20, 2003.
(60) Provisional application No. 60/494,841, filed on Aug. 12, 2003.

(51) Int. Cl. A61K 48/00; A61L 9/04; A61K 9/14; A61K 39/00

(52) U.S. Cl. 424/46; 424/185.1; 514/44

(57) ABSTRACT

The present invention discloses compositions and methods for treating lung diseases. In preferred embodiments the methods involve administering to the subject via a pulmonary, oropharyngeal, or nasopharyngeal route a compound or composition that contains a therapeutic agent and a targeting element directed to a ligand. The ligand is preferably an epitope on pIgR receptor.

### RELATED APPLICATIONS

This application claims the benefit of U.S. Provisional Application No. 60/439,373, filed Jan. 9, 2003, 60/480,047 filed Jun. 20, 2003, and 60/494,841 filed Aug. 12, 2003, the contents of each of which are incorporated herein in their entirety.

### FIELD OF THE INVENTION

The present invention relates to the field of compositions and methods for treating lung diseases.

### BACKGROUND OF THE INVENTION

The following description of the background of the invention is provided simply as an aid in understanding the invention and is not admitted to describe or constitute prior art to the invention.

Lung diseases comprise a spectrum of manifestations and etiologies, and may be particularly difficult to treat with systemic administration of potential therapeutics. Broad categories of disease classifications exemplify this spectrum of lung diseases. Over 150 diseases of the interstitium are recognized, including many types of fibrosis. Another category includes disorders of gas exchange and blood circulation. Disorders of the airways and disorders of the pleura constitute two additional categories. Lung cancers include both primary lung cancers and metastases from primary cancers of various other organs or tissues. Infectious diseases include viral, bacterial, and fungal infectious agents.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. However, not all low molecular weight drugs can be efficaciously administered through the lung. Moreover, pulmonary delivery of higher molecular weight therapeutics, such as polypeptides or proteins, is much more difficult.

The anatomy and physiology of the lung presents several barriers to pulmonary administration. Initially, after passing through the nose or mouth, inhaled air (and any particles contained therein) moves into the respiratory tree, which is composed of numerous dichotomous branches between the trachea and the alveoli. Bronchi, bronchioles, and terminal bronchioles comprise the conducting zone. The epithelium of these conducting airways is pseudo stratified and largely ciliated. The more distal levels of branching form the transitional and respiratory zones, comprised of respiratory bronchioles, alveolar ducts, and alveoli, is where gas exchange and pulmonary absorption occur. The respiratory zone, in contrast to the conducting zone, is non-ciliated and comprised of a single cell layer.

The air-blood barrier is comprised of the alveolar epithelium, the capillary endothelium, and the lymph-filled interstitial space separating these two cell layers. In the alveolar epithelium, adjacent cells overlap and are bound by non-leaky tight junctions, which, in conjunction with the non-leaky single cell layer comprising the capillary endothelium, limits the movement of fluids, cells, salts, proteins, and numerous other macromolecules from the blood and intercellular spaces into the lumen of the alveoli. Most molecules, including proteins and polypeptides, must be actively or passively transported across this barrier in the absence of lung injury. Also, mucosal secretions from epithelial cells and cilia provide additional physical barriers to the delivery of a potential therapeutic.

Other cell types present in the alveolar lumen and in the interstitial space separating the alveolar epithelium from the capillary endothelium may also serve as barriers for delivery. Alveolar macrophages migrate from the blood across the air-blood barrier. Additionally, other cell types, such as neutrophils and lymphocytes, can move into the alveoli from the blood in response to infection.

Immunotherapy directed to tumor-associated or tumor-specific antigens has long been considered an attractive method for safe, nontoxic treatment of tumors. Translating such methods into clinical benefit, however, has been somewhat less successful than might have been hoped. While many tumors express antigens that could be used to generate an in vitro or in vivo immune response, direct targeting of such antigens may not be the most effective mode of providing immunotherapy. Cytokines, such as interleukin-2 (“IL-2”), have also been employed to stimulate immune response to tumors. Such therapies, either alone or with conventional therapies, may provide a more attractive means for achieving clinical benefit in malignant and non-malignant diseases. See, e.g., Xu et al., Cancer Res. 60: 4475-84 (2000); Christ et al., Clinical Cancer Res. 7: 1385-97 (2001); Steven A. Rosenberg, The Transformed Cell: Unlocking the Mysteries of Cancer, Putnam Group, 1992.

Experimental treatment of certain tumors with cytokines has been performed by various artisans. Cytokines, such as IL-2, have been administered systemically (e.g., by intravenous infusion and/or subcutaneous administration), with the demonstration of some antitumor response. However, serious side effects have also been observed in such treatments, including fever, pulmonary vascular leakage, weight gain, malaise, rigor, anemia, and thrombocytopenia. See, e.g., Heinzer et al., J. Clin. Oncol. 17: 3612-20 (1999). More recently, aerosol delivery of cytokines such as IL-2 have been shown to provide reduced toxicity coupled with modest therapeutic benefit. See, e.g., Lorenz et al., Clin. Cancer. Res. 2: 1115-22 (1996); Zissel et al., Cancer Immunol. Immunother. 42: 122-26 (1996); Khanna et al., J. Pharm. Pharmacol. 49: 960-71 (1997).

Acute respiratory infections can affect both the upper or lower respiratory systems. An upper respiratory infection typically involves the ears, nose, throat or sinuses. Examples of upper respiratory tract infections include the common cold (typically viral); the flu (influenza virus); otitis media, pharyngitis, acute sinusitis or chronic sinusitis, and tonsillitis, which involve inflammation of the middle ear, throat, sinuses, and tonsils, respectively. Lower respiratory infections typically involve the trachea, bronchial tubes and the lungs themselves. Examples of lower respiratory tract infections include bronchitis and pneumonia. In a single infection, one or both of the upper and lower respiratory systems can be affected.

Respiratory tract infections are primarily of bacterial, viral, or fungal origin; although there are also rarer types, such as parasitic infections. Pulmonary tuberculosis (TB) is an example of a contagious bacterial infection caused by Mycobacterium tuberculosis. The lungs are primarily involved, but the infection can spread to other organs. TB is one of the most clinically significant infections worldwide, with an incidence of 3 million deaths and 10 million new cases each year. With improved sanitary conditions and the advent of antimicrobial drugs, the incidence of mortality had been steadily declining. However, in most developed countries, there has been a resurgence of TB infection, in part due to immunocompromised individuals (e.g., HIV-positive) and the emergence of multidrug-resistant (MDR) strains of M. tuberculosis.

Severe acute respiratory syndrome (SARS) is a newly recognized viral respiratory tract infection, first detected in China in late 2002. The viral agent has been identified as a previously unrecognized human coronavirus, called SARS-associated coronavirus (SARS-CoV). SARS is also an example of both upper and lower respiratory tract involvement caused by infection with a single organism. Early symptoms include runny nose and sore throat, which are then followed by dyspnea and dry cough, and may develop into adult respiratory distress syndrome requiring intervention with mechanical ventilation.

Pneumonia is an example of a respiratory tract that may be caused by either bacteria, viruses, or parasites. It is generally defined as an inflammation of the lung tissue, whereby white cells in the lungs prevent the alveoli from functioning properly. This condition is potentially life-threatening.

Candida and Aspergillus are the most common fungal respiratory tract infections, tending to appear in immunocompromised subjects, such as transplant recipients. While Candida mainly infests the upper tracheobronchial tree with only an occasional chance of dissemination, Aspergillus has the potential to involve the deeper parenchyma. Other potential fungal pathogens include Cryptococcus, Pseudallerscheria and Coccidioides.

Experimental treatment of certain infections with cytokines has also been performed by various artisans. Cytokines have been used to treat serious bacterial and viral infections (particularly, those caused by drug resistant organisms), either alone or in combination therapies with known treatments or vaccines. For a review of immune modulation in the treatment of respiratory infection, the reader is referred to Kolls and Nelson, Resp. Res. 1:9-11, 2000. For example, tuberculosis, the seventh leading cause or morbidity and mortality in the world, has been successfully treated with recombinant interferon-γ in aerosol form (Condos et al., Lancet 349:1513-5, 1997). As another example, intranasal interferon-α 2b has been shown to prevent rhinovirus infection, and to lessen symptoms associated with parainfluenza infections (Monto et al., J. Infect. Dis. 154:128-133, 1986). Other examples of therapeutic molecules for the treatment of infections include chemokines such as gamma-interferon-inducible protein 10 (IP-10), interferon-inducible T cell alpha chemoattractant (I-TAC) and MIG (monokine induced by interferon-gamma). Antibodies directed against a variety of epitopes of infectious agents causing infection are also known in the art, for both treatment and prevention (e.g., vaccines) of infection.

To achieve maximum therapeutic impact in the treatment of any lung disease, potential therapeutic agents should be optimally directly delivered to the respiratory tract. A number of general methods have been described for delivering medically important molecules, including small molecules, nucleic acids, and/or protein or peptide compositions, in an effort to improve bioavailability and/or to target delivery to particular locations within the body. Such methods include the use of prodrugs, encapsulation into liposomes or other particles, co-administration in uptake enhancing formulations, and targeting to specific tissues. For review see, e.g., Critical Reviews in Therapeutic Drug Carrier Systems, Stephen D. Bruck, ed., CRC Press, 1991. In the case of cytokines such as IL-2, pulmonary delivery has relied upon both inhalation of free cytokine (either alone or in combination with intravenous delivery of additional cytokine), and inhalation of liposomal formulations. See, e.g., Enk et al., Cancer 88: 2042-46 (2000); Khanna et al., J. Pharm. Pharmacol. 49: 960-71 (1997). Such delivery modes can provide high cytokine levels within the lung, but relatively modest systemic cytokine levels.

Certain modes for delivering medically important molecules (e.g., oral, nasopharyngeal, oropharyngeal, pulmonary, buccal, sublingual, mucosal, vaginal, or rectal delivery modes) require that the molecule(s) of interest be delivered across “polarized” cells (e.g., epithelial cells) that have two distinct surfaces. In the case of pulmonary epithelium, these surfaces are referred to as the apical surface, which is exposed to the aqueous or gaseous medium in which the molecule(s) of interest is delivered to the subject; and the opposing basolateral (also known as basal lateral) side that rests upon and is supported by an underlying basement membrane, and that can provide access to the interstitial spaces and the general circulation. Tight junctions between adjacent epithelial cells separate the apical and basolateral sides of an individual epithelial cell. The biological methods that provide and maintain such cellular polarity can also act to limit bioavailability of molecules delivered by these modes.

Molecules are trafficked into, out of, and within a cell by various means, and it is typically these means that are believed to confer bioavailability to a molecule delivered by oral, nasopharyngeal, oropharyngeal, pulmonary, buccal, sublingual, mucosal, vaginal, or rectal delivery modes. “Active transport” is a general term for the energy-dependent carriage of substances across a cell membrane. “Endocytosis” is a general term for the process of cellular internalization of molecules, i.e., processes in which cells take in molecules from their environment, either passively or actively. “Exocytosis” is a general term for processes in which molecules are passively or actively moved from the interior of a cell into the medium surrounding the cell. “Transcytosis” is a general term for processes in which molecules are transported from one surface of a cell to another. “Paracytosis” is a general term for processes in which molecules are transferred through the interstices between cells, often past tight junctions. “Receptor mediated endocytosis” refers to a particular type of trafficking event by which cells internalize molecules, viruses, bacteria, etc. As its name implies, it depends on the interaction of that molecule with a specific binding protein in the cell membrane called a “receptor.” “Forward transport” refers to transport in a basolateral to apical direction, while “reverse transport” refers to transport in an apical to basolateral direction.

Each publication and patent application in the foregoing Background section is hereby incorporated by reference in its entirety, including all tables, figures, and claims.

### SUMMARY OF THE INVENTION

The present invention discloses methods of treating lung diseases. The methods involve administering to a subject via a pulmonary, oropharyngeal, or nasopharyngeal route a compound or composition that contains a therapeutic agent and a targeting element directed to a ligand present on the surface of cells lining the pulmonary or nasopharyngeal system. The ligand preferably confers transcytosis of the compound or composition across polarized epithelial layers, either in vitro or in vivo. The therapeutic agent is preferably a cytokine or a chemokine, more preferably an interleukin or an interferon, IP-10, I-TAC, or MIG. The therapeutic agent may also be an antibody, for example, an antibody directed against an infectious agent. The invention is described herein in detail with regard to targeting elements that target an epitope on pIgR receptor. In particularly preferred embodiments, the targeting element confers apical to basolateral transcytosis to the therapeutic agent in an in vitro transcytotic assay. The subject is preferably a human that is, for example, diagnosed with a lung disease and in need of treatment, or predisposed to a lung disease and in need of prophylaxis.

In various embodiments, exemplary ligands include one or more of the following: pIgR, pIgR stalk, transferrin receptor, apo-transferrin, holo-transferrin, vitamin B12 receptor, FcRn, an integrin, Flt-1, Flk-1, Flt-4, a GPI-linked protein, a scavenger receptor, folate receptor, and low density lipoprotein receptor. In the most preferred embodiment, the ligand is pIgR or the pIgR stalk. In preferred embodiments, the targeting element binds a non-secretory component region of pIgR. In additional embodiments, the therapeutic agent is a polypeptide, preferably an enzyme, a cytokine or a chemokine. In various embodiments, the therapeutic agent is one or more of the following: an enzyme, an interleukin, an interferon, a cytokine, a chemokine or an antibody. The following list of interleukins is not inclusive and is provided by way of example only. Other interleukins, those existing and those yet to be discovered, are also contemplated for use in the invention. However, an exemplary list of interleukins includes any of IL-1, IL-2, IL-3, IL-4, IL-5, IL-6, IL-7, IL-9, IL-10, IL-12, IL-13, IL-15, IL-18, IL-21, and functional derivatives of any of these foregoing exemplary interleukins. Likewise, the following list of interferons is not inclusive and is provided by way of example only. An exemplary list of interferons include interferon α (including interferon alpha-2a and -2b), interferon β, and interferon γ. In the most preferred embodiments, the interleukin is IL-2, or a functional derivative thereof, and the interferon is interferon α or interferon β, or a functional derivative thereof of either. Preferred chemokines include IP-10, I-TAC and MIG. Combinations of any two or more cytokines, chemokines, or other therapeutic agents are also provided herein.

The term “functional derivative” as used herein refers to a chemically modified version, an analog, or a homolog of a compound that retains a biological function of interest of that compound for any given application. In the case of polypeptides, chemical modification may include, by way of non-limiting example, adding chemical groups to a compound (e.g., glycosylation, phosphorylation, thiolation, pegylation, acetylation, amidation, glycosylphosphoinositolyzation, etc.), eliminating parts of a compound that do not impact the function of interest (preparing a truncated form of a protein that retains an activity of interest, e.g., Klenow fragment), extending a compound with sequences that add domains or functions to the compound (e.g., preparing fusion proteins); changing sets of one or more amino acids in the polypeptide (preparing muteins). In preferred embodiments, functional derivatives of therapeutic compounds described herein extend the residence time of the therapeutic compound in the lungs, for example, by slowing their release or metabolism.

Analogs are exemplified by peptidomimetics; and homologs are polypeptides from other species of animals that retain biological activity (e.g., human and porcine insulin, human and salmon calcitonin, etc.) or intraspecies isomers of a polypeptide (protein “families” such as the cytochrome P450 family). Muteins and pegylated functional derivatives of IL-2, for example, are well known to those of skill in the art. See, e.g., Chapes et al., J. Appl. Physiol. 86: 2065-76 (1999); Shanafelt et al., Nature Biotechnol. 18: 1197-202 (2000). IL-2 biological activity of the functional derivatives are preferably tested by evaluating the ability to sustain proliferation of the IL-2-dependent murine cytotoxic T cell line, CTLL-2. See, e.g., Melani et al., Cancer Res. 58:4146-54 (1998). Likewise, functional derivatives of IL-2 linked to Fc or human serum albumin are well known in the art. See, e.g., Zheng et al., J. Immunol. 163: 4041-48 (1999); Melder et al., Modulation of anti-infective responses in mice by Albuleukin, an Interleukin-2/human serum albumin fusion protein, Society for Biological Therapy Meeting. November 2001.

By “pulmonary route” is meant administration of a compound or composition to a subject through the airways leading to the lungs. The pulmonary route includes, but is not limited to, all passageways including the trachea, larynx, bronchioles, bronchus, and alveoli.

The “nasopharynx” refers to any of the nasal passages, pharynx, trachea, and larynx. By a “nasopharyngeal route” is meant that the compound enters the subject through the nasopharynx. Similarly, the “oropharynx” refers to the oral cavity, and includes the back of the tongue (base of tongue), soft palate, tonsils and its pillars, and the back wall of the throat (posterior pharyngeal wall), through the pharynx, trachea, and larynx. Thus, by an “oropharyngeal route” is meant that the compound enters the subject through any one or more of the membranes of the oropharynx. In various embodiments the mode of administration is instillation, nebulization, aerosolization, atomization, misting, or inhalation, and most preferably inhalation.

The pharynx stretches from the back of the nose, down the neck to the larynx. The trachea connects the larynx to the bronchial tubes. The larynx is a structure of muscle and cartilage in the upper neck that contains the vocal cords. Air passes through the larynx into the windpipe and then into the lungs.

Preferred delivery methodologies of the present invention include instillation, or inhalation of a material generated by nebulization, aerosolization, atomization, and misting. “Instillation” refers to direct delivery of liquid in liquid drops to a pulmonary passageway. “Inhalation” is the most preferably form of administration and refers to inhaling gas (preferably air) that contains the compound into the lungs and/or naso-pharynx of the subject, preferably by force of the subject's own respiration. “Nebulization” refers to creating a fine spray or mist of particles from liquid. “Aerosolization” refers to creating a suspension of fine solid or liquid particles in gas. “Atomization” refers to reducing the composition to fine particles or spray.

An “anti-tumor agent” is an agent that destroys, shrinks, or arrests the growth of tumors or cancers in a subject, or that extends the life of a subject receiving the agent. The skilled artisan will understand that anti-tumor agents do not necessarily produce an anti-tumor effect in each subject receiving the agent. Rather, whether or not an agent destroys, shrinks, or arrests the growth of tumors or cancers in a subject, or that extends the life of a subject is a statistical question measured in a population receiving the treatment, which is compared to a like population not receiving the treatment. Preferably, an anti-tumor agent extends the average life span of a subject by 3 months, 6 months, 9 months, 1 year, 2 years, 3 years, 5 years, or more, relative to a subject not receiving the treatment. In particularly preferred embodiments, an anti-tumor agent reduces the average incidence or average time to appearance of metastatic disease in a subject, most preferably lung metastases, relative to a subject not receiving the treatment.

In certain embodiments, the anti-tumor agent may be an anti-angiogenesis agent. An “anti-angiogenesis agent” is a compound that blocks or prevents the function of an angiogenic factor that normally promotes the development of a tumor's blood supply. Tumor angiogenesis is the specific development of an adequate blood supply for a solid tumor mass; and the growth of a tumor depends upon the existence, maintenance, and continued development of sufficient and functional blood vasculature in the tumor mass. Tumor angiogenesis thus involves endothelial cell penetration of the vascular basement membrane in a preexisting blood vessel; followed by endothelial cell proliferation; and then by an invasion of the extracellular matrix surrounding the blood vessel to form a newly created vascular spout (see, e.g., Vernon and E. H. Sage, Am. J. Pathol. 147: 873-883 (1995).

An “angiogenic factor” as used herein, refers to a compound that promotes angiogenesis. Such factors include, for example, vascular endothelial growth factors (VEGFs) and VEGF receptors, fibroblast growth factors (FGFs), transforming growth factor (TGF) α and β, platelet-derived endothelial cell growth factor (PD-ECGF), tumor necrosis factor-α(TNF-α), matrix metalloproteinases (MMPs), angiopoietin-2 and Tie-2 receptor, scatter factor (hepatocytes growth factor, IL-8, angiogenin, adhesion molecules (e.g., integrins, selecting, cadherins), prostaglandin E1 and E2, angiogenin transforming growth factors, angiotropin, granulocyte-colony stimulating factor, placental growth factor, and proliferin.

Anti-angiogenesis agents may thus block the normal function of one of these angiogenesis agents, for example, an antibody directed against VEGF. Alternatively, there are natural anti-angiogenesis agents, or anti-angiogenetic factors, which normally balance the angiogenesis agents in vivo. Anti-angiogenetic factors include angiostatin, endostatin, IFN-α and IFN-β, IFN-γ inducible protein 10, IL-1, IL-6, IL-12, platelet factor 4, thrombospondin-1,2-methoxyoestradiol, tissue inhibitors of metalloproteinases, retinoic acid, prolactin, basic fibroblast growth factor soluble receptor, transforming growth factor-# (TGF-β, placental proliferin-related protein, TNF-α, I-TAC and MIG. The therapeutic agents of the invention may comprise such anti-angiogenesis agents, or may be administered in combination with such anti-angiogenesis agents as a second therapeutic agent.

In certain embodiments, the therapeutic agent may be an apoptosis inducer. Apoptosis, which is also referred to as programmed cell death, is a form of cell death characterized by membrane blebbing and nuclear DNA fragmentation. Dysregulation of apoptosis has been implicated in a number of human diseases, including cancer. Although apoptotic cell death is initially triggered by a specific death signal received, for example, by ligation of the Fas cell surface molecule, execution of the apoptotic pathway occurs only upon the activation of members of the Ced-3/ICE (caspase) family of cysteine proteases. There are at least 10 known members of the caspase family whose activities lead to site-specific cleavage and consequent activation/inactivation of various target molecules. FLICE and related caspases may initiate apoptosis by activating a downstream caspase cascade, including CPP32 (caspase-3). The decision to engage the apoptotic execution pathway in response to specific death signals depends on the status of various cellular regulators of apoptosis, including p53 and the Bcl-2/Bax set point. The latter set point arises through heterodimerization between the Bcl-2/Bcl-X_{L} family of suppressors and promoters, respectively, in which the ratio of the heterodimerizing partners determines the outcome, cell death or cell survival, in response to various death signals. Bad, a more distantly related family member, is a direct regulator of the set point, by a mechanism that is governed by phosphorylation. The phosphorylation may, in turn, be affected by Bcl-2-dependent recruitment of Raf-1 kinase. Thus, an “apoptosis inducer” as used herein, is a molecule that interacts with an apoptotic pathway to trigger cell death, or blocks the function of another molecule that prevents apoptosis. The therapeutic agents of the invention may comprise such apoptosis inducers, or may be administered in combination with such apoptosis inducers as a second therapeutic agent.

An “anti-infective agent” is an agent that prevents infection by an infectious agent, decreases the severity of infection by an infectious agent, interferes with normal infection pathways, arrests infection by an infectious agent, impairs the function of growth of an infectious agent, or kills an infectious agent. The skilled artisan will understand that anti-infective agents do not necessarily produce an anti-infective effect in each subject receiving the agent. Rather, whether or not an agent is effective is a statistical question measured in a population receiving the treatment, which is compared to a like population not receiving the treatment.

A “ligand,” “target molecule” or “molecular target” is a compound, a molecular complex of two or more compounds, a moiety (a portion of a compound), or an interface formed between two or more compounds, that are associated with a cell surface and to which a targeting element specifically binds. Preferred ligands are membrane proteins, most preferably pIgR, pIgR stalk, transferrin receptor, apo-transferrin, holo-transferrin, vitamin B 12 receptor, FcRn, an integrin, Flt-1, Flk-1, Flt-4, a GPI-linked protein, a scavenger receptor, folate receptor, and/or low density lipoprotein receptor.

The term “targeting element” encompasses any type of composition or compound that is capable of specifically binding to a molecular target. The term “specifically binds” is not intended to indicate that the targeting element binds exclusively to its intended target. Rather, a targeting element specifically binds if its affinity for its intended target is about 2-fold greater when compared to its affinity for a non-target molecule. Preferably the affinity of the targeting element will be at least about 5-fold, preferably 10-fold, more preferably 25-fold, even more preferably 50-fold, and most preferably 100-fold or more, greater for a target molecule than its affinity for a non-target molecule. A compound or composition comprising such a targeting element would be referred to as being “adapted to specifically bind” to the target molecule. Preferred targeting elements can be selected from the group consisting of a polypeptide, a recombinant polypeptide, an antibody, an antibody fragment, a single-chain variable region fragment, a small molecule, an oligonucleotide, an oligosaccharide, a polysaccharide, a carbohydrate, a cyclic polypeptide, a peptidomimetic, and an aptamer, as these terms are defined herein.

A cell surface component is said to “promote” transport, active transport, endocytosis, or transcytosis if a compound or composition comprising a targeting element that specifically binds to the cell surface component is transported into, around, or through a cell (depending on the type of transport involved) at a higher rate or to a higher absolute amount compared to a similar composition lacking the targeting element. Preferably, a 2-fold, 5-fold, 10-fold, 100-fold, or 1000-fold increase in rate or amount is obtained.

The term “compound” as used herein refers to a single covalently linked molecule. Preferably, a compound comprises one or more therapeutic agents covalently linked to one or more targeting elements.

The term “composition” as used herein refers to a plurality of compounds associated by non-covalent means. A composition may include a compound comprising one or more therapeutic agents covalently linked to one or more targeting elements, associated with pharmaceutically acceptable excipients. Alternatively, a composition may refer to one or more therapeutic agents and one or more targeting elements associated with a particle or capsule as described in the entirety of Provisional U.S. Patent Application No. 60/402,029, filed Aug. 7, 2002, which is hereby incorporated by reference.

As used herein, the term “small molecule” refers to compounds having molecular mass of less than 3000 Daltons, preferably less than 2000 or 1500, still more preferably less than 1000, and most preferably less than 600 Daltons. Preferably but not necessarily, a small molecule is not an oligopeptide.

As used herein, the term “polypeptide” refers to a covalent assembly comprising at least two monomeric amino acid units linked to adjacent amino acid units by amide bonds. An “oligopeptide” is a polypeptide comprising a short amino acid sequence (i.e., 2 to 10 amino acids). An oligopeptide is generally prepared by chemical synthesis or by fragmenting a larger polypeptide. Examples of polypeptide drugs include, but are not limited to, therapeutic antibodies, insulin, parathyroid hormone, polypeptide vaccines, and antibiotics such as vancomycin. Novel polypeptide drugs may be identified by, e.g., phage display methods.

As used herein, the term “antibody” refers to a molecule comprising at least one antigen binding domain formed by two binding regions referred to by those of skill in the art as an immunoglobulin or immunoglobulin-like heavy chain, and an immunoglobulin or immunoglobulin-like light chain. When obtained by in vitro or in vivo generation of an immunogenic response, the heavy and light chains are expressed as separate polypeptides, and are joined by disulfide bonds. In this case, the heavy and light chains may be separated under reducing conditions. Such antibodies include both polyclonal, monospecific and monoclonal antibodies, and antigen binding fragments thereof (e.g., Fab fragments, Fab′ fragments, etc.). An “immunogenic response” is one that results in the production of antibodies directed to one or more proteins after the appropriate cells have been contacted with such proteins, or polypeptide derivatives thereof, in a manner such that one or more portions of the protein function as epitopes.

When a molecule comprising at least one antigen binding domain is formed recombinantly, the heavy and light chains may be linked by disulfide bonds as in the foregoing discussion. However, in various embodiments, the heavy and light chains are linked by non-reducible covalent linkers. As used herein, the term “single-chain variable region fragment” or “sFv” refers to a variable, antigen-binding determinative region of a single antibody light chain and antibody heavy chain linked together by a covalent linkage having a length sufficient to allow the light and heavy chain portions to form an antigen binding site. Such a linker may be as short as a covalent bond; preferred linkers are from 2 to 50 amino acids, and more preferably from 5 to 25 amino acids. The antigen binding site need not be formed from intramolecular association of light and heavy chain portions; rather, two separate sFvs may form multimeric antigen binding molecules (e.g. diabodies) as described hereinafter.

As used herein, the term “polynucleotide” refers to molecule comprising a covalent assembly of nucleotides linked typically by phosphodiester bonds through the 3′ and 5′ hydroxyls of adjacent ribose units. An “oligonucleotide” is a polynucleotide comprising a short base sequence (i.e., 2 to 10 nucleotides). Polynucleotides include both RNA and DNA, may assume three-dimensional shapes such as hammerheads, hairpins, dumbbells, etc., and may be single or double stranded. Polynucleotide drugs can include ribozymes, and polynucleotide vaccines.

As used herein, the term “oligonucleotide analog” refers to a molecule that mimics the structure and function of an oligonucleotide, but which is not a covalent assembly of nucleotides linked by phosphodiester bonds. Peptide nucleic acids, comprising purine and pyrimidine bases linked via a backbone linkage of N-(2-aminoethyl)-glycine units, is an example of an oligonucleotide analog.

As used herein, a “carbohydrate” is any form of saccharide. Examples of carbohydrates include, but are not limited to, simple sugars or oligosaccharides (such as monosaccharides, disaccharides, etc. which have typical molecular weights less than 1000) as well as macromolecular (polymeric or polysaccharides) substances such as starch, glycogen, and cellulose polysaccharides (which may have molecular weights on the order of 10⁵-10⁶). The term “polysaccharide” as used herein refers to a carbohydrate comprising 2 or more covalently-linked saccharide units. An “oligosaccharide” is a polysaccharide comprising a short saccharide sequence (i.e., 2 to 10 saccharide units).

As used herein, the term “cyclic polypeptide” refers to a molecule comprising a covalent assembly of monomeric amino acid units, each of which is linked to at least two adjacent amino acid units by amide bonds to form a macrocycle.

As used herein, the term “peptidomimetic” refers to a molecule that mimics the structure and function of a polypeptide, but which is not a covalent assembly of amino acids linked by amide bonds. A peptoid, which is a polymer of N-substituted glycine units, is an example of a peptidomimetic.

The term “aptamer” as used herein refers to polynucleotides that bind to non-polynucleotide target molecules (e.g., a polypeptide or small molecule).

The term “immune system modulator” as used herein refers to a natural or recombinant molecule that is normally produced by and/or manifests its effects through cells of the immune system.

“Interleukin” is the generic name for a group of well-characterized cytokines that are produced by leukocytes and other cell types (e.g., endothelial cells, monocytes, fibroblasts, and dendritic cells). Interleukins have a broad spectrum of functional activities that regulate the activities and capabilities of a wide variety of cell types. They are particularly important as members of the cytokine networks that regulate inflammatory and immune responses.

Cytokines represent a vast array of relatively low molecular weight, pharmacologically active proteins that are secreted by one cell for the purpose of altering either its own functions (autocrine effect) or those of adjacent cells (paracrine effect). In many instances, individual cytokines have multiple biological activities. Different cytokines can also have the same activity, which provides for functional redundancy within the inflammatory and immune systems.

The term “cytokine” as used herein is considered to include amino acid sequence, glycosylation and other variants of the native molecules. These variants may exhibit enhanced levels of the normal biological activity of the native molecules or may, on the contrary, act antagonistically towards the native molecule. Alternatively, variants are selected for improved characteristics such as stability to oxidation, extended biological half-life, and the like. Such variants as are known or will be developed in the future are suitable for use herein.

Interleukins are the cytokines that act specifically as mediators between leucocytes. The following table shows the major source and effects of some types of interleukins.

| IL | Major source | Major effects |
|---|---|---|
| IL-1 | Macrophages | Stimulation of T cells |
| | | and antigen-presenting cells. |
| | | B-cell growth and antibody |
| | | production. Promotes hematopoiesis |
| | | (blood cell formation). |
| IL-2 | Activated T cells | Proliferation of activated T cells. |
| IL-3 | T lymphocytes | Growth of blood cell precursors. |
| IL-4 | T cell and mast cells | B-cell proliferation. |
| | | IgE production. |
| IL-5 | T cells and mast cells | Eosinophil growth. |
| IL-6 | Activated T cells | Synergistic effects |
| | | with IL-1 or TNFα. |
| IL-7 | thymus and bone marrow | Development of T cell |
| | stromal cells | and B cell precursors. |
| IL-8 | Macrophages | Chemoattracts neutrophils. |
| IL-9 | Activated T cells | Promotes growth of |
| | | T cells and mast cells. |
| IL-10 | Activated T cells, B cells | Inhibits inflammatory and |
| | and monocytes | immune responses. |
| IL-11 | Stromal cells | Synergistic effects |
| | | on hematopoiesis. |
| IL-12 | Macrophages, B cells | Promotes T_{H}1 cells |
| | | while suppressing T_{H}2 functions |
| IL-13 | T_{H}2 cells | Similar to IL-4 effects |
| IL-15 | Epithelial cells and | Similar to IL-2 effects. |
| | monocytes | |
| IL-16 | CD8 T cells | Chemoattracts CD4 T cells. |
| IL-17 | Activated memory T cells | Promotes T cell proliferation. |
| IL-18 | Macrophages | Induces IFNγ production. |

Interferons (IFNs) are a class of cytokines or cell signaling proteins with immune stimulating/modulating activity, involved in activating cellular immunity to infections. The interferons are a family of small proteins and glycoproteins with molecular weights of approximately 15,000 to 27,600 daltons (about 15-27 kDa) produced and secreted in vivo by cells primarily in response to viral infection, and also in response to synthetic or biological inducers. Advancing knowledge and technology have shown various interferons to be produced by the same cell types (one basis for nomenclature), the discovery of different species and forms of interferon, and the discovery that some forms are identical to others previously reported. There are three major classes, IFN-α(alpha or alfa), IFN-β(beta), and IFN-γ(gamma).

Interferons exert their cellular activities by binding to specific membrane receptors on the cell surface. Once bound to the cell membrane, interferons initiate a complex sequence of intracellular events, including the up-regulation of certain other cytokines, induction of certain enzymes, suppression of cell proliferation, immunomodulating activities such as enhancement of the phagocytic activity of macrophages and augmentation of the specific cytotoxicity of lymphocytes (cellular immunity) for target cells, and inhibition of virus replication in virus-infected cells. IFNs have been used to treat various respiratory disorders, including respiratory tract and lung infections, such as multidrug-resistant pulmonary tuberculosis.

Interferon products currently approved and marketed in the U.S. include: a) one natural (human cell-derived) α-interferon product, Interferon alfa-n3 (Human Leukocyte Derived) or Alferon N Injection; b) three forms of recombinant α-interferons—Interferon alfa-2b (Intron A), Interferon alfa-2a (Roferon A), and Interferon alfacon-1 or Infergen; c) three forms of recombinant β-interferons—Interferon beta-1b or Betaseron and Interferon beta-1 a (e.g., Avonex or Rebif); and d) one γ-interferon—Interferon gamma-1b or Actimmune. A natural α-interferon, Interferon alfa-n1, Lymphoblastoid or Wellferon, was approved in 1999 but was abandoned before market launch in the U.S. Additionally, two different forms of pegylated recombinant α-interferon are awaiting FDA approval or have recently been approved, both for treatment of chronic hepatitis C—Peginterferon alfa-2b or PEG-INTRON from Schering-Plough Corp. and Peginterferon alfa-2a or Pegasys from Hoffmann-La Roche Inc. Pegylation involves attachment of inert polyethylene glycol (PEG) polymer side chains to the interferon molecules to improve their pharmacokinetic properties (extend their half-lives).

“Natural” (cell culture-derived) interferon products, which contain a multiplicity of interferon types or species, are considered by some to provide potentially better therapeutic efficacy than single-species recombinant interferon products. For example, natural α-interferon can be used at a four-times lower dosage to treat condyloma (genital warts) than recombinant interferon a products. Natural α-interferons are generally produced by intentional virus infection stimulation of human lymphoblastoid or leukocyte cells, with purification by chromatographic and electrophoretic techniques. Native human β-interferon is generally produced by superinducing human fibroblast cultures with poly-IC (polyriboinosinic acid-polyribocytidylic acid polymer), a well-documented inducer of interferon expression, with isolation and purification by chromatographic and electrophoretic techniques.

β-interferon products are currently approved only for multiple sclerosis indications. β-interferon may act by multiple pathways in MS: regulation of T-cell functions such as activation, proliferation and suppressor cell function; modulation of the production of cytokines; down-regulation of proinflammatory cytokines and interferon gamma; up-regulation of inhibitory anti-inflammatory cytokines; regulation of T-cell migration and infiltration into the central nervous system via the blood brain barrier.

The nomenclature of interferon products is complex. It has changed over time and different conventions (or none) and descriptors are often used to refer to the same or different molecules. According to one classic approach, there were three classes of interferon: leukocyte, fibroblast, and immune interferon. These are loosely named for their source, e.g., secreted by leukocyte or fibroblast cells or in response to viral or other immune challenge. It was originally presumed that cells secreted only one type of interferon. However, it is now known that interferon-expressing cells can produce multiple types of interferon and multiple subtypes (subspecies, e.g., alpha-2a or alpha-2b). Multiple interferon subspecies of each major species/type have been identified, e.g., interferon alpha-2a and interferon alpha-2b. Two major classes of interferons have been identified (i.e., type-I and type-II; according to one classification scheme). All type-I interferons share common biological activities generated by binding of interferon to the cell-surface receptor, leading to the production of several interferon-stimulated gene products. Type-I interferons include a family of more than 25 types (species) of interferon α as well as interferon beta and interferon ω species. All currently approved interferon products are type I. Type-I interferons induce pleiotropic biologic responses which include antiviral, anti-proliferative and immunomodulatory effects, regulation of cell surface major histocompatibility antigen (HLA class I and class II), and induction and regulation of other cytokine expression. Examples of interferon-stimulated gene products include 2′5′ oligoadenylate synthetase (2′5′ OAS) and beta-2 microglobulin.

A newer, more commonly used, nomenclature system is based on initial characterization of the types of interferon produced by different cell types. For example, over 25 species of α-interferons are produced by macrophages and B-, non-B- and non-T-lymphocytes. This nomenclature uses Greek letters, e.g., a (for leukocyte and lymphoblastoid cell interferon), β (for fibroblast interferon), and γ (for immune interferon), along with numbers or small Roman letters designating subspecies (often named in the order in which they were identified). The term ‘alpha’ or ‘alfa’ may be used when referring to commercial α-interferon products, e.g., in FDA proper names. Within each interferon class, interferons share considerable homology, i.e., their nucleotide and amino acid sequences are very similar. One source (U.S. Pat. No. 5,676,942) reports the equivalence of the following alpha interferon species term: aA, a2a, aM, a4a; a2b; a2c, a4b; aB, a8a, aMI, a4a; aB, a8c; aB2, a8b, aN, a14c; aC, a10a, aO, a16; aD, a1a, aI, a17a; a1b, aI′, a17b; a5, 88, or a17c; aH, a14a, aI1, a17d; aJ, a7a, af, a21a; aJ1, a7c; aJ2, a7b, a(Ovch);a21b; aK, a6. While all interferons within an interferon species (e.g., α, β, γ) have similar biological effects, not all the activities are shared by each interferon subspecies in that class. In many cases, the extent of activity varies substantially for each interferon subspecies (e.g., α2a, α2b). Both natural (human cell-derived) and recombinant interferon products are embraced by the present invention.

Chemokines are chemotactic cytokines that are important regulators of leukocyte-mediated inflammation and immunity. Chemokines have been grouped into four major categories (see table below), according to the number and arrangement of conserved N-terminal cysteine motifs: C, CC, CXC, and CX3C, where “X” is a nonconserved amino acid. The CXC chemokines and CC chemokines are the largest families with each member containing four cysteine residues. Most chemokines are 8-10 kDa in size, cationic at neutral pH, and share 20-70% amino acid sequence homology. CXC chemokines are further subdivided into two classes based on the presence or absence of a tripeptide motif Glu-Leu-Arg (ELR), N-terminal to the conserved CXC region. Members that contain the motif (ELR+) are potent chemoattractants for neutrophils and promoters of angiogenesis, whereas those that do not contain the motif (ELR−) are potent chemoattractants for mononuclear cells, and the group that is inducible by interferon-gamma are potent inhibitors of angiogenesis.

Most chemokines form dimers, which dissociate upon dilution into biologically active monomers. Chemokine activities are mediated by seven-transmembrane-domain G protein coupled receptors. Chemokines have been identified to play a role in angiogenesis and tumor inhibition, and as HIV-suppressive factors by interacting with chemokine receptors which, together with CD4, were recognized as the binding sites for HIV-1. In addition, a variety of chemokines have been shown to display defensin-like antimicrobial activities.

Defensins are a family of antimicrobial and cytotoxic peptides (about 29-35 amino acid residues in length) including six invariant cysteines creating a triple-stranded beta-sheet configuration structure. Defensins are known to be anti-infective agents against gram positive and gram negative bacteria, fungi, and some enveloped viruses. Defensins have also been shown to be cytotoxic against a wide range of normal and malignant targets. They appear to function by inserting and permeabilizing cell membranes. Two major classes have been identified, alpha and beta-defensins. Alpha-defensins are produced by neutrophils and intestinal Paneth's cells. Beta-defensins are mainly produced by epithelial cells. Alpha-Defensins are present in the airway secretions of patients with various chronic inflammatory lung disorders, and have been shown to be cytotoxic toward airway epithelial cells and to induce chemokine secretion in several cell types.

The following table shows representative chemokines that are commercially available (R&D Systems, Minneapolis, Minn.).

| | | Human | Human | Mouse | Mouse | |
|---|---|---|---|---|---|---|
| Systematic Name | SCY Name | Ligand | Aliases | Ligand | Aliases | Receptor |
| C FAMILY | | | | | | |
| XCL1 | SCYC1/2 | Lptn | SCM-1, | Lptn | | XCR1 |
| | | | ATAC | | | |
| XCL2 | SCYC1/2 | SCM-1β | | | | XCR1 |
| CX₃C | | | | | | |
| FAMILY | | | | | | |
| CX3CL1 | | Fractalkine | ABCD-3 | Neurotactin | | CX3CR1 |
| CC FAMILY | | | | | | |
| CCL1 | SCYA1 | I-309 | | TCA-3 | P500, I- | CCR8 |
| | | | | | 309 | |
| CCL2 | SCYA2 | MCP-1 | MCAF, | JE? | | CCR2 |
| | | | LDGF, | | | |
| | | | GDCF, | | | |
| | | | TDCF, | | | |
| | | | SMC-CF, | | | |
| | | | HC11, | | | |
| | | | TSG8 | | | |
| CCL3 | SCYA3 | MIP-1α | LD78α, | MIP-1α | GOS19, | CCR1, |
| | | | LD78β, | | LD78α | CCR5 |
| | | | GOS19, | | | |
| | | | Pat464 | | | |
| CCL4 | SCYA4 | MIP-1β | pAT744, | MIP-1β | pAT744, | CCR5 |
| | | | ACT-2, | | ACT-2, | |
| | | | G-26, | | G-26, | |
| | | | HC21, | | HC21, | |
| | | | H400, | | MAD-5, | |
| | | | MAD-5, | | LAG-1 | |
| | | | LAG-1 | | | |
| CCL5 | SCYA5 | RANTES | | RANTES | | CCR1, |
| | | | | | | CCR3, |
| | | | | | | CCR5 |
| CCL6 | SCYA6 | ? | | C10 | MRP-1 | ? |
| CCL7 | SCYA7 | MCP-3 | NC28, | MARC | NC28, | CCR1, |
| | | | FIC, | | FIC | CCR2, |
| | | | MARC | | | CCR3 |
| CCL8 | SCYA8 | MCP-2 | HC-14 | MCP-2? | | CCR3 |
| CCL9/10 | SCYA9/10 | ? | | MIP-1γ | MRP-2, | ? |
| | | | | | CCF18, | |
| | | | | | C10-like | |
| CCL11 | SCYA11 | Eotaxin | | Eotaxin | | CCR3 |
| CCL12 | SCYA12 | ? | | MCP-5* | | CCR2 |
| CCL13 | SCYA13 | MCP-4 | Ck β10, | | | CCR2, |
| | | | NCC-1 | | | CCR3 |
| CCL14 | SCYA14 | HCC-1 | MCIF, Ck | | | CCR1 |
| | | | β1, NCC- | | | |
| | | | 2, HCC-3 | | | |
| CCL15 | SCYA15 | MIP-1δ, | CC-2, | | | CCR1, |
| | | Lkn-1 | MIP-5, | | | CCR3 |
| | | | HCC-2, | | | |
| | | | CCF-18, | | | |
| | | | NCC-3 | | | |
| CCL16 | SCYA16 | HCC-4 | LEC, | | | CCR1 |
| | | | ILINK, | | | |
| | | | NCC-4, | | | |
| | | | LEC, | | | |
| | | | LMC, CK | | | |
| | | | β12 | | | |
| CCL17 | SCYA17 | TARC | Dendrokine, | TARC | Dendrokine, | CCR4, |
| | | | ABCD-2 | | ABCD-2 | CCR8 |
| CCL18 | SCYA18 | PARC | DC-CK1, | | | ? |
| | | | AMAC-1, | | | |
| | | | MIP-4, | | | |
| | | | Dctactin | | | |
| CCL19 | SCYA19 | MIP-3β | ELC, | MIP-3β | ELC, | CCR7 |
| | | | Exodus-3, | | Exodus-3, | |
| | | | Ck β11 | | Ck β11 | |
| CCL20 | SCYA20 | MIP-3α | LARC, | MIP-3α | LARC, | CCR6 |
| | | | Exodus-1, | | Exodus-1, | |
| | | | Mexikine, | | Mexikine, | |
| | | | ST38, CK | | ST38, CK | |
| | | | β4 | | ββ4 | |
| CCL21 | SCYA21 | 6Ckine | Exodus-2, | 6Ckine | Exodus-2, | CCR7 |
| | | | SLC, | | SLC, | |
| | | | TCA4, | | TCA4, | |
| | | | CK β9 | | CK ββ9 | |
| CCL22 | SCYA22 | MDC | STCP-1, | MDC | ABCD-1, | CCR4 |
| | | | DCtactin | | DCtactin | |
| | | | β, ABCD- | | β, STCP- | |
| | | | 1, DC/B- | | 1, DC/B- | |
| | | | CK | | CK | |
| CCL23 | SCYA23 | MPIF-1, | Ck β8, | | | CCR1 |
| | | Ck β8-1 | MIP-3 | | | |
| CCL24 | SCYA24 | Eotaxin-2, | MPIF-2, | Eotaxin-2 | MPIF-2, | CCR3 |
| | | | Ck βv6 | | Ck βv6 | |
| CCL25 | SCYA25 | TECK | | TECK | | CCR9 |
| CCL26 | SCYA26 | Eotaxin-3 | Finetaxin, | | | CCR3 |
| | | | TMkine, | | | |
| | | | IMAC | | | |
| CCL27 | SCYA27 | CTACK | ILC, | CTACK | ALP, ILC, | CCR10 |
| | | | PESKY, | | Eskine, | |
| | | | Eskine | | PESKY, | |
| | | | | | skinkine | |
| CCL28 | SCYA28 | | | | | CCR10? |
| CXC | | | | | | |
| FAMILY | | | | | | |
| CXCL1 | SCYB1 | GROα | MGSA-α, | | | CXCR2 > CXCR1 |
| | | | GRO-1, | | | |
| | | | NAP-3 | | | |
| CXCL2 | SCYB2 | GROβ | MGSA-β, | MIP-2? | | CXCR2 > CXCR1 |
| | | | MIP-2α, | | | |
| | | | GRO-2 | | | |
| CXCL3 | SCYB3 | GROγ | MGSA-γ, | | | CXCR2 > CXCR1 |
| | | | MIP-2β, | | | |
| | | | GRO-3 | | | |
| CXCL4 | SCYB4 | PF4 | | PF4 | | ? |
| CXCL5 | SCYB5 | ENA-78 | AMCF-II | LIX? | | CXCR2, |
| | | | | | | CXCR1 |
| CXCL6 | SCYB6 | GCP-2 | CKα3 | | | CXCR1, |
| | | | | | | CXCR2 |
| CXCL7 | SCYB7 | NAP-2 | MDGF | | | CXCR2 |
| CXCL8 | SCYB8 | IL-8 | NCF, | | | CXCR1, |
| | | | NAP-1, | | | CXCR2 |
| | | | MDNCF, | | | |
| | | | LUCT, | | | |
| | | | AMCF-1, | | | |
| | | | MONAP | | | |
| CXCL 9 | SCYB9 | MIG | | MIG | | CXCR3 |
| CXCL10 | SCYB10 | IP-10 | | CRG-2 | IP-10 | CXCR3 |
| CXCL11 | SCYB11/ | I-TAC | b-R1, | I-TAC | | CXCR3 |
| | 9B | | H174, IP-9 | | | |
| CXCL12 | SCYB12 | SDF-1α/β | PBSF, | SDF-1 | PBSF, | CXCR4 |
| | | | hIRH, | | TLSR-α, | |
| | | | TLSR-α/β, | | TPAR1 | |
| | | | TPAR1 | | | |
| CXCL13 | SCYB13 | BLC/BCA-1 | CXC-X, | BLC/BCA-1 | CXC-X, | CXCR5 |
| | | | BLR1L, | | BLR1L, | |
| | | | Angie | | Angie | |
| CXCL14 | SCYB14 | BRAK | CXC-X3, | BRAK, | CXC-X3, | ? |
| | | | Bolekine, | BMAC | Bolekine, | |
| | | | NJAC | | NJAC | |
| CXCL15 | SCYB15 | | CINC-2β- | Lungkine | Weche | ? |
| | | | like | | | |

I-TAC, interferon-inducible protein 10 (IP-10) and monokine induced by gamma interferon (MIG) are CXC ELR− chemokines and bind to the CXCR3 receptor. Each is a potent anti-angiogenic factor and chemoattractant for T-cells (Th1) activated by IL-2, but not for unstimulated T-cells. I-TAC has the highest affinity for CXCR3, making it the dominant ligand to CXCR3 and more potent than IP-10 or MIG as a chemoattractant (Neote et al., J Exp Med. 1998 Jun. 15;187(12):2009-21).

CXC ELR+ chemokines include interleukin-8 (IL-8), which binds to CXCR1 and CXCR2. IL-8 is a chemoattractant for neutrophils and is a potent inducer of angiogenesis.

Th1 and Th2 provide various roles in the immune system. The Th phenotypes are characterized by the cytokines they produce (see table below).

| Phenotype | Cytokines Produced |
|---|---|
| Th1 | IFN-γ, TNF-β, IL-2, IL-10 |
| Th2 | IL-4, IL-5, IL-6, IL-13, IL- |
| | 10 |

Th1 and Th2 cells are associated with specific immune responses due to the cytokines they secrete. In the case of Th1-type cytokines, IFN-γ promotes phagocytosis and upregulates microbial killing. In particular, it induces IgG 2A (in mice) which is known to opsonize bacteria. IFN-γ provides all the tools necessary to eliminate most external microbes. IL-4 is the classic Th2 cytokine; its secretion triggers a number of events that parallel those of IFN-γ. IL-4 promotes production of neutralizing antibodies (IgG) and the mast cell/eosinophil degranulating antibody known as IgE. It also promotes upregulation of IgE receptors on mast cells, eosinophils and macrophages. IL-4 and IFN-γ often exist in an antagonistic relationship. IFN-γ blocks IgE and IgG1 production, while IL-4 blocks IgG2A secretion.

Th1 cells preferentially express CCR5 and CXCR3. Th2 cells preferentially express CCR4, CCR8 and, to a lesser extent, CCR3. Therefore, it appears to be possible to selectively induce the migration of Th1 and Th2 cells. Th1 cells are involved in cell-mediated immunity and associated with autoimmune disorders and allograft rejection. Th2 cells are involved in mediating allergic inflammation and chronic fibroproliferative disorders; these include asthma, atopic dermatitis, idiopathic pulmonary fibrosis and systemic fibrosis. A disease scenario may occur where the inciting agent may induce an unsuccessful Th1 response, and the subsequent host reaction may favor a response dominated by Th2 cytokines. This is one way to induce fibrosis. Shifting the chemokine balance toward CXC ELR− chemokines to restore the Th1 response by administering I-TAC may be effective at treating the particular fibroproliferative disorder.

The term “GPI-linked protein” as used herein refers to a class of eukaryotic proteins that have a glycosylphosphoinositol lipid (GPI) modification at the carboxy-terminal end. The GPI moiety, added post-translationally to proteins in the endoplasmic reticulum in vivo, that serves as a means of membrane anchoring of a protein to the external plasma membrane. In polarized cells, such as MDCK cells, GPI-linked proteins are preferentially segregated to the apical cell surface, where they may be associated with microdomains known as “rafts.” Rafts, and their GPI-linked contents, can be internalized under certain conditions, such as by antibody-induced crosslinking of GPI-linked proteins. At least a portion of these internalized rafts may be transcytosed by the polarized cells. See, e.g., Verkade et al., J. Cell Biol. 148: 727-39 (1999); Muniz and Riezman, EMBO J. 19: 10-15 (2000).

The term “scavenger receptor” as used herein refers to a class of proteins that mediates the uptake of modified forms of lipoproteins, including low density lipoproteins (“LDL”). Cell types such as macrophages, endothelial cells, intestinal epithelial cells, and smooth muscle cells have been shown to have scavenger receptors for modified lipoproteins, and the scavenger receptor family has grown to include cell surface receptors which mediate cholesterol transport by ‘scavenging’ cholesterol from HDL. Scavenger receptors also bind a range of polyanionic ligands other than modified lipoproteins. See, e.g., Platt and Gordon, Chem. Biol. 5: R193-203 (1998); Werder et al., Biochemistry 40: 11643-50 (2001); Zingg et al., Arterioscler. Thromb. Vasc. Biol. 22: 412-17 (2002).

A polyimmunoglobulin receptor (pIgR) molecule has several structurally and functionally distinct regions that are defined as follows. In the art, a pIgR molecule is generally described as consisting of two different, loosely defined regions called the “stalk” and the “secretory component” (SC). When performing its intended biological function, a pIgR molecule binds polymeric immunoglobulins (IgA or IgM) on the basolateral side, and then transports the immunoglobulin to the apical side. Proteolyic cleavage of pIgR takes place on the apical side of an epithelial cell between the SC and the stalk. The SC molecule is released from the cellular membrane and remains bound to and protects the immunoglobulins, whereas the stalk molecule remains bound to the cellular membrane (see “Mucosal Immunoglobulins” by Mestecky et al. in: Mucosal Immunology, edited by P. L. Ogra, M. E. Lamm, J. Bienenstock, and J. R. McGhee, Academic Press, 1999). Domains of a pIgR molecule that are of particular interest in the present disclosure include but are not limited to domain 5, domain 6, the B region, the stalk, the transmembrane domain, the secretory component, and the intracellular domain.

Particularly preferred pIgR molecules are those described in U.S. Pat. No. 6,042,833, and the simian pIgR described in U.S. patent application Ser. No. 60/266,182 (attorney docket No. 057220.0701) entitled “Compositions and Methods for Identifying, Characterizing, Optimizing and Using Ligands to Transcytotic Molecules” by Houston, L. L., and Sheridan, Philip L., which was filed on Feb. 2, 2001. However, it is understood that, in the context of this invention, pIgR also refers to any of that receptor's family or superfamily members, any homolog of those receptors identified in other organisms, any isoforms of these receptors, any pIgR-like molecule, as well as any fragments, derivatives, mutations, or other modifications expressed on or by cells such as those located in the respiratory tract, the gastrointestinal tract, the urinary and reproductive tracts, the nasal cavity, buccal cavity, ocular surfaces, dermal surfaces and any other mucosal epithelial cells. Preferred pIgR and pIgR-like proteins are those that direct the endocytosis or transcytosis of proteins into or across epithelial cells. pIgR is part of the very large immunoglobulin superfamily. The extracellular, IgA binding part of the molecule contains 5 Ig-like domains.

As used herein, the terms “secretory component” and “SC” refers to the smallest (shortest amino acid sequence) portion of an apical proteolyzed pIgR molecule that retains the ability to bind immunoglobulins (IgA and IgM). After proteolytic cleavage of pIgR, some amino acid residues remain associated with SC:immunoglobulin complexes but are eventually degraded and/or removed from such complexes (Ahnen et al., J. Clin. Invest. 77:1841-1848, 1986). According to the definition of the secretory component used herein, such amino acids are not part of the SC. In certain embodiments of the invention, pIgR-targeting elements that do not recognize or bind to the SC are preferred.

As used herein, the term “stalk” refers to a molecule having an amino acid sequence derived from a pIgR, wherein the stalk sequence does not comprise amino acid sequences derived from the SC. A stalk molecule comprises pIgR amino acid sequences that remain bound to the apical membrane following the apical proteolytic cleavage when such cleavage occurs and pIgR amino acid sequences required for such cleavage. Preferred stalk molecules confer one or more transcytotic properties to a ligand bound thereto. Most preferred are stalk molecules that confer the ability to undergo apical to basolateral transcytosis to a compound or composition (e.g., ligand) bound thereto.

In various embodiments, the lung disease may be lung cancer, a respiratory tract or lung infection, a disease of the interstitium, a disorder of gas exchange or blood circulation, a disease of the airways or a disorder of the pleura. As used herein, a “lung cancer” refers to either a primary lung tumor (for example, bronchogenic carcinoma or bronchial carcinoid) or a metastasis from a primary tumor of another organ or tissue (for example, breast, colon, prostate, kidney, thyroid, stomach, cervix, rectum, testis, bone, or melanoma). As used herein, a “respiratory tract or lung infection” refers to any bacterial, viral, fungal, or parasite infection of any part of the respiratory system. As used herein, a “disease of the interstitium” includes any disorder of the interstitium including fibrosis (for example, interstitial pulmonary fibrosis, interstitial pneumonia, interstitial lung disease, Langerhans' cell granulomatosis, sarcoidosis, or idiopathic pulmonary hemosiderosis). As used herein, a “disorder of gas exchange or blood circulation”, refers to any abnormality affecting the distribution and/or exchange of gases to/from the blood and lungs (for example, pulmonary edema, pulmonary embolism, respiratory failure (e.g., due to weak muscles), acute respiratory distress syndrome, or pulmonary hypertension). As used herein, a “disease of the airway” includes any disorder of regular breathing patterns, including disorders of genetic and environmental etiologies (for example, asthma, chronic bronchitis, bronchiolitis, cystic fibrosis, bronchiectasis, emphysema, chronic obstructive pulmonary disease, diffuse panbronchiolitis, or lymphangiomyonatosis). As used herein, a “disorder of the pleura” includes, for example, pleural effusion (e.g., hemothorax (blood into the pleural space), or emphysema (pus into the pleural space), pneumothorax (air, e.g., traumatic, spontaneous, or tension), pleurisy or pleural fibrosis or calcification.

In preferred embodiments, the compound is administered through inhalation in a form such as liquid particles and/or solid particles (e.g., an aerosol, a nebula, a mist, an atomized sample, liquid drops, etc.). The compound or a therapeutic portion thereof is preferably delivered into the lung with a pharmacokinetic profile that results in the delivery of an effective dose of the compound or a therapeutic portion thereof. In preferred embodiments at least 1%, more preferably at least 5%, even more preferably at least 10%, still more preferably at least 20%, and most preferably at least 30% or more of the administered compound or a therapeutic portion or metabolite thereof preferably undergoes apical to basolateral transcytosis from the pulmonary lumen.

An “effective dose” or a compound or therapeutic agent of the invention is that amount which is able to treat a lung disease, reverse the progression of a lung disease, halt the progression of a lung disease, or prevent the occurence of a lung disease in a subject to whom the compound or therapeutic agent is administered, as compared to a matched subject not receiving the compound or therapeutic agent.

An “effective dose of an anti-tumor compound or agent” is an amount of compound that is capable of killing cancer cells, preventing expansion of the size of a cancer or tumor mass, delay or prevent appearance of metastatic disease, or extend the lifespan of a subject. For example, in one embodiment an effective dose shrinks the size of a cancer or tumor mass. In another embodiment an effective dose kills cancer cells that have metastasized to a treated area and/or prevents the cells from forming a metastatic mass.

In certain embodiments, the tumor in a subject is a primary tumor, most preferably of the lung; however, more preferably the tumor in a subject is a secondary tumor, and most preferably is a pulmonary metastasis from a primary tumor that is not of the lung. In various embodiments the primary tumor is selected from the group consisting of a sarcoma, an adenocarcinoma, a choriocarcinoma, and a melanoma. In other embodiments, the tumor is a colon adenocarcinoma, a breast adenocarcinoma, an Ewing's sarcoma, or an osteosarcoma. In the most preferred embodiment, the primary tumor is a renal cell carcinoma and the secondary tumor is a tumor of the lung. In various embodiments, the clinical presentation of the pulmonary metastasis is a solitary metastasis, a cannonball, a lymphangitis carcinoimatosa, or a pleural effusion. A “primary” tumor is the original tumor in a subject. A “secondary” tumor is a cancer that has metastasized from the organ in which it first appeared to another organ.

An “effective dose of an anti-infective compound or agent” is an amount of anti-infective compound that prevents infection by an infectious agent, decreases the severity of infection by an infectious agent, interferes with normal infection pathways, arrests infection by an infectious agent, impairs the function of growth of an infectious agent, or kills an infectious agent. The infectious agent may be a bacteria, a virus, a fungus, a parasite, or any other agent that causes local or systemic infection. Preferably, the infection is a respiratory tract infection or an infection of the lung. In certain embodiments, the infection is a bacterial infection, for example, causing tuberculosis. In other embodiments, the infection is a viral infection, for example, causing severe acute respiratory syndrome (SARS). In other embodiments the infection is a fungal infection. In yet other embodiments, the infection may be caused by multiple types of infectious agents, for example, pneumonia.

The amount of a therapeutic compound that is effective as defined above may change under additional embodiments, wherein the compound is used in combination therapy. As used herein, “combination therapy” refers to the administration of more than one therapeutic compound, either sequentially or simultaneously. In certain embodiments, invention compounds comprising a first therapeutic agent may be administered in combination therapy with a second therapeutic agent, either formulated as another invention compound, or unmodified. In other embodiments, invention compounds comprising a first therapeutic agent may be administered in combination therapy with a vaccine, for example, directed against an infective agent, a cancer-causing agent, or a cancer-associated polypeptide.

In preferred embodiments the targeting element binds to an epitope on pIgR or the pIgR stalk that comprises an amino acid sequence selected from the following: LRKED, QLFVNEE, LNQLT, YWCKW, GWYWC, STLVPL, SYRTD, QDPRLF and KRSSK. In more preferred embodiments the targeting element binds to pIgR or the pIgR stalk in a region selected from the following:
R1 KRSSK to the carboxy terminus of pIgR;
R2a From SYRTD to the carboxy terminus of pIgR,
R2b From SYRTD to KRSSK,
R3a From STLVPL to the carboxy terminus of pIgR,
R3b From STLVPL to KRSSK,
R3c From STLVPL to SYRTD,
R4a From GWYWC to the carboxy terminus of pIgR,
R4b From GWYWC to KRSSK,
R4c From GWYWC to SYRTD,
R4d From GWYWC to STLVPL,
R5a From YWCKW to the carboxy terminus of pIgR,
R5b From YWCKW to KRSSK,
R5c From YWCKW to SYRTD,
R5d From YWCKW to STLVPL,
R5e From YWCKW to GWYWC,
R6a From LNQLT to the carboxy terminus of pIgR,
R6b From LNQLT to KRSSK,
R6c From LNQLT to SYRTD,
R6d From LNQLT to STLVPL,
R6e From LNQLT to GWYWC,
R6f From LNQLT to YWCKW,
R7a From QLFVNEE to the carboxy terminus of pIgR,
R7b From QLFVNEE to KRSSK,
R7c From QLFVNEE to SYRTD,
R7d From QLFVNEE to STLVPL,
R7e From QLFVNEE to GWYWC,
R7f From QLFVNEE to YWCKW,
R7g From QLFVNEE to LNQLT,
R8a From LRKED to the carboxy terminus of pIgR,
R8b From LRKED to KRSSK,
R8c From LRKED to SYRTD,
R8d From LRKED to STLVPL,
R8e From LRKED to GWYWC,
R8f From LRKED to YWCKW,
R8g From LRKED to LNQLT, and
R8h From LRKED to QLFVNEE.

In additional embodiments the compound can also contain a second targeting element, which can be substantially identical to the first targeting element. While targeting elements may have a single binding site for a ligand (e.g., as in a monomeric sFv), in preferred embodiments, the targeting element has two to four binding sites for the ligand, and more preferably the targeting element is selected from the following: an antibody, an Fab fragment, and a single chain variable region fragment (sFv) diabody. Alternatively, the second targeting element can be different from the first targeting element.

In other embodiments the targeting element has two to four single chain variable region fragments (sFv), each sFv having a heavy chain variable domain covalently linked, directly or through a polypeptide linker, to a light chain variable domain. The sFvs are covalently or noncovalently associated with the therapeutic agent. In preferred embodiments, at least one sFv binds to pIgR, and more preferably to a non-secretory component region of pIgR, and most preferably binds to pIgR stalk. In various embodiments the targeting element can be a monoclonal antibody, or a fragment of an antibody, which includes a Fab fragment, an sFv fragment, or a fragment of the variable region of an antibody. sFv antibody fragments can be conveniently expressed in E. coli and purified by chromatographic separation.

In a related aspect, the complexes and compounds of the invention further comprises a PTD or MTS. “Protein transduction domains” (PTD) and “membrane transport signals” (MTS) are polypeptides, typically about 10-35 amino acids long, that facilitate, promote or induce the uptake of proteins and other polypeptides by cells. The PTD are derived from HIV-TAT, HSV-VP22 and Antenapedia (the source of Penetratin), and are characterized by having a high content of positively charged arginine (Arg) and lysine (Lys) residues. The MTS are very hydrophobic peptides derived from secretory signal sequences, which partition into the hydrophobic layer of a membrane lipid bilayers.

In additional aspects, the present invention relates to devices configured and arranged for pulmonary delivery of the compounds or compositions described herein. Such devices comprise one or more compounds or compositions dispersed in an appropriate medium for delivery by inhalation or instillation. Most preferably, the device is a nebulizer or an inhaler. Such devices for delivery of medicaments are well known to those of skill in the art. See, e.g., U.S. Pat. Nos. 6,488,027, 6,453,900, 6,427,688, 6,427,683, 6,415,784, 6,338,443, 6,076,519, 5,906,198, and 5,653,223, each of which is hereby incorporated by reference in its entirety, including all tables figures and claims.

The summary of the invention described above is not limiting and other features and advantages of the invention will be apparent from the following detailed description of the preferred embodiments, as well as from the claims.

### BRIEF DESCRIPTION OF THE DRAWINGS

FIG. 1 provides a schematic illustration of an sFv domain structure, and a model of the interactions between sFvs forming a dimeric “diabody” structure.

FIG. 2 provides a graphical illustration of the plasma concentration of sFv obtained by intra-tracheal instillation of dimeric sFv diabodies in Cyno monkeys (1 mg/kg with protease inhibitors).

FIG. 3 provides the plasma concentration of sFv obtained by aerosol delivery to Cynomolgus monkeys as a function of time after inspiration an tidal volumes of 75% and 40% of vital capacity.

FIG. 4 provides a comparison of plasma concentrations of sFv obtained by aerosol, instillation, and IV delivery routes as a function of time after delivery.

FIG. 5 depicts the coding sequence of an exemplary pIgR-directed sFv (APL10).

FIG. 6 depicts the coding sequence of an exemplary pIgR-directed sFv-IL-2 fusion protein.

FIG. 7 provides maps of exemplary IL-2-sFv expression constructs.

### DETAILED DESCRIPTION OF THE INVENTION

Recombinant human cytokines and chemokines are powerful mediators of diverse cell functions, mainly, but not exclusively, within the immune system. As a result, they represent an attractive approach to the management of cancer and infectious disease. Interleukin-2 (IL-2), the best explored and most frequently used of these cytokines, is one of the most important interleukins presently used in clinical practice. Interleukin-2 is used with patients that have advanced renal cell carcinoma, metastatic malignant melanoma, and acute non-lymphoblastic leukemia. Similarly, α-interferon is used for treatment of tumors such as hairy cell leukemia, AIDS-related Kaposi's sarcoma, multiple myeloma, chronic myelogenous leukemia, bladder carcinoma, non-Hodgkin's lymphoma, colorectal carcinoma, cutaneous T-cell lymphoma, follicular lymphoma, renal cell carcinoma and malignant melanoma.

A major disadvantage of interleukin therapy is the multiorgan toxicity. Metastatic kidney cancer is a life-threatening disease, and interleukin-2 is useful in patients with this disease. Interleukin-2 is more effective with higher dose administrations. Yet toxicity due to interleukin-2 is often a very serious problem. Administration of interleukin-2 is often accompanied by co-administration of agents designed to ameliorate the toxic effects. Similarly, α-interferon therapy may cause or aggravate fatal or life-threatening neuropsychiatric, autoimmune, ischemic, and infectious conditions.

It would be a great advantage to have a mode of administering medications that reduce such toxic side effects, while still providing a medically effective cytokine dose. Such a mode of administration would allow treated subjects to benefit from cytokine and chemokine therapies, and other therapies involving such drugs, while being shielded from any harmful effects. Further, such a technology could be extended to utilize even non-toxic drugs at higher doses than otherwise administrable.

The present invention provides versatile treatment methods for delivery of therapeutic agents, including cytokines. In one embodiment the methods can be used to treat a subject that may be exposed to or has a lung disease, with the goal of either preventing or treating the lung disease. Because the present invention describes methods for providing locally high concentrations of an therapeutic agent in the interstitial spaces or blood vessels of the lung, the invention is preferably applied where the disease or disorder has spread to the lung tissue.

In certain preferred embodiments, methods can be used to treat a subject that has a primary tumor, either with or without the presence of a secondary tumor, with the object of preventing or delaying a secondary tumor from developing, of extending life expectancy, and/or of reducing the size of an existing primary or secondary tumor. Because the present invention describes methods for providing locally high concentrations of an anti-tumor agent in the interstitial spaces or blood vessels of the lung, the invention is preferably applied where the primary or secondary tumor is a tumor of the lung. Most preferably, the invention is applied where the primary tumor is a renal cell carcinoma.

In other preferred embodiments, the invention is applied where the lung has been subjected to bacterial infection, for example, causing tuberculosis, or viral infection, for example, causing SARS.

Because the present invention can also provide significant bioavailability of an therapeutic agent in the general circulation, the present invention can also be utilized in methods of treating tumors of the body, other than the lung, and systemic infection that has spread beyond the respiratory tract as well. The methods can be employed to place an therapeutic agent into the bloodstream, which is carried to other parts of the body where a tumor or an infective agent is present. Targeting elements can be employed to achieve apical to basolateral transcytosis across the pulmonary, nasopharyngeal, or oropharyngeal epithelium. Additional targeting elements can also be present on the compound or composition which will target the actual site of infection.

Exemplary Lung Cancers and Metastases

While the following cancerous conditions are provided for purposes of example, the methods, compositions, and devices described herein may be used for treatment of lung cancers and metastases of primary tumors of other organs or tissues to the lung generally.

Stage IV metastatic melanoma is a disease that generally has a fatal outcome, with survival times averaging less than 1 year. A particularly common problem in metastatic melanoma is lung metastasis, which occurs in 30-50% of Stage 1V cases. Metastasis to the lungs often causes respiratory problems that severely limit the subject's quality of life. Pulmonary delivery of IL-2 in metastatic melanoma, together with traditional chemotherapy, has been disclosed. See, e.g., Enk et al., Cancer 88: 2042-46 (2000).

Renal cell carcinoma is the most common tumor rising from the kidney, with about 30,000 cases per year diagnosed in the United States. Diagnosed early as a small tumor confined to the kidney, this disease may be cured by surgery. However, most cases of renal cell carcinoma are not diagnosed until a later developmental stage and approximately 30% of patients with renal carcinoma present with metastatic disease. While more than 50% of patients with renal cell carcinoma are cured in early stages, the outcome for stage IV disease is poor. The Robson staging system is used to describe the stages of disease and is as follows:
Stage I—Tumor confined within capsule of kidney.
Stage II—Tumor invading perinephric fat but still contained within the Gerota fascia.
Stage III—Tumor invading the renal vein or inferior vena cava (A), or regional lymph-node involvement (B), or both (C).
Stage IV—Tumor invading adjacent viscera (excluding ipsilateral adrenal) or distant metastases.

The probability of cure is related directly to the stage or degree of tumor dissemination. Effective treatment can improve symptoms and survival in a proportion of patients using immunotherapy, radiation therapy, or surgery in certain cases. Chemotherapy drugs are largely ineffective for renal cell carcinoma, and are rarely used by themselves. Immunotherapy drugs, on the other hand, show modest activity against renal cell carcinoma. Immunotherapy drugs used against renal cell carcinoma include interleukin-2, interferon-alpha, and interferon-gamma. Selected patients with metastatic disease respond to immunotherapy, but many patients can be offered only palliative therapy. See, e.g., Huland et al., J. Urology 147: 344-48 (1992); Huland et al., Cancer J. Sci. Am. 3: S98-S105 (1997); Huland et al., Anticancer Res. 19: 2679-84 (1999).

Lung cancer is the uncontrolled growth of abnormal cells in one or both of the lungs. While normal lung tissue cells reproduce and develop into healthy lung tissue, these abnormal cells reproduce rapidly and never become normal lung tissue. Masses of cancer cells (tumors) then form and disrupt the lung, making it difficult to function properly.

More than 87% of lung cancers are smoking related. However, not all smokers develop lung cancer. Quitting smoking reduces an individual's risk significantly, although former smokers remain at greater risk for lung cancer than people who never smoked. Exposure to other carcinogens such as asbestos and radon gas also increases an individual's risk, especially when combined with cigarette or cigar smoking.

Non-small cell lung cancer (NSCLC) has an imbalance in expression of ELR+ (angiogenic) and ELR− (angiostatic) CXC chemokines that favors angiogenesis and tumor growth. The ELR+ chemokines, such as IL-8, are elevated, while the ELR− chemokines (1-TAC, IP-10 and MIG) remain at normal levels, suggesting that the ELR− chemokines are not at levels that can counter regulate the ELR+ chemokines. Investigators have demonstrated that administering IP-10 or MIG in a SCID mouse model with NSCLC inhibits tumor growth.

Exemplary Infectious Diseases and Infectious Agents

While the following infectious diseases and infectious agents are provided for purposes of example, the methods, compositions, and devices described herein may be used for treatment of infection generally.

Mycobacterium tuberculosis is an intracellular pathogen that infects macrophages. Most inhaled bacilli are destroyed by activated alveolar macrophages. However, the surviving bacilli can multiply in macrophages and be released upon cell death, which signals the infiltration of lymphocytes, monocytes and macrophages to the site. Lysis of the bacilli-laden macrophages is mediated by delayed-type hypersensitivity (DTH) and results in the development of a solid caseous tubercle surrounding the area of infected cells. Continued DTH causes the tubercle to liquefy, thereby releasing entrapped bacilli. The large dose of extracellular bacilli triggers further DTH, causing damage to the bronchi and dissemination by lymphatic, hematogenous and bronchial routes, and eventually allowing infectious bacilli to be spread by respiration.

Anti-infective agents that are used to treat TB include, for example, isoniazid, rifampin, pyrazinamide, ethambutol, and streptomycin. Chemoprophylaxis is highly effective and generally consists of isoniazid at a dose of 300 mg/day for 6 to 9 months for adults. For children, the dosage is 10 mg/kg/day, up to 300 mg, given as a single morning dose.

Pseudomonas aeruginosa causes chronic respiratory infections and is the leading cause of high morbidity and mortality in cyctic fibrosis (CF). The initially colonizing P. aeruginosa strains are nonmucoid, but in the lung of a CF patient they begin to produce mucoid, which leads to the inability of patients to clear the infection, even under aggressive antibiotic therapies. The emergence of the mucoid form of P. aeruginosa is associated with further disease deterioration and poor prognosis. P. aeruginosa is also the second most common cause of infections in intensive care units, and a frequent cause of pneumonias. HIV-infected patients are also at risk.

Several penicillins, including ticarcillin, piperacillin, mezlocillin, and azlocillin, are active against Pseudomonas. Other anti-infective agents include, for example, ceftazidime, cefepime, aztreonam, imipenem, meropenem, and ciprofloxacin. Ticarcillin is used most often at dosages of 16 to 20 g/day IV. Piperacillin, azlocillin, cefepime, ceftazidime, meropenem, and imipenem are active in vitro against some strains resistant to ticarcillin.

Bacillus anthracis, the causative agent of anthrax, is a large, Gram-positive, facultatively anaerobic, encapsulated rod. The spores resist destruction by disinfectants and heat and remain viable in soil and animal products for decades. Human infection occurs usually through the skin, rarely in the GI tract, and inhalation of spores may result in potentially fatal pulmonary anthrax.

An anthrax vaccine, composed of a culture filtrate, is available for those at high risk (armed forces personnel, veterinarians, laboratory technicians, employees of textile mills processing imported goat hair). Repeated vaccination may be required to ensure protection and local reactions to the vaccine itself can occur.

Most strains of anthrax are susceptible to penicillin. However, the organism often manifests inducible beta-lactamases, so single-drug therapy with penicillin or cephalosporins is not recommended. Prophlaxis upon exposure requires oral ciprofloxacin 500 mg bid, or doxycycline 100 mg bid for 60 days; or amoxicillin 500 mg tid. Induction of beta-lactam resistance is of less concern with the lower number of organisms present in prophylactic use. Pulmonary anthrax is frequently fatal, but survival is possible with early treatment and intensive pulmonary and circulatory support. Corticosteroids may be useful but have not been adequately evaluated.

Pneumonia is a condition is caused by a wide variety of bacteria, viruses, fungi, and other types of organisms that infect the respiratory tract. Infectious agents may enter through the mouth and reach the lung during respiration. Smoking contributes to pneumonia since it damages the cilia lining the respiratory tract. Malnutrition or conditions like kidney failure or sickle cell disease also impair the lung's ability to get rid of microorganisms that cause pneumonia. Moreover, viral infections of the upper respiratory tract can predispose a person to pneumonia by also damaging the protective cilia.

Among children 12 and under, the most frequent cause of pneumonia is the pneumococcus bacterium. Among adolescents and young adults, the most frequent infective agent is a bacteria-like microbe called Mycoplasma pneumoniae.

Bacterial pneumonia can also ensue as a complication of influenza A; secondary infections are most often caused by Streptococcus pneumoniae, Haemophilus influenzae, or (most serious of all) Staphylococcus aureus.

The following table presents organisms associated with various pneumonias.

| Bacteria | Viruses |
|---|---|
| Streptococcus pneumoniae | Influenza |
| Streptococcus pyogenes (Grp A) | Parainfluenza |
| Streptococcus agalactiae (Grp B) | Cytomegalovirus |
| Staphylococcus aureus | Adenovirus |
| Bacillus anthracis | Epstein-Barr Virus |
| Other Bacillus sp. | Herpes Simplex Virus |
| Nocardia sp. | Varicella-Zoster |
| Enterobacteriaceae | Coxsackievirus |
| Pseudomonas aeruginosa | Measles |
| Acinetobacter sp. | Rhinovirus |
| Burkholderia pseudomallei | Respiratory Syncytial Virus |
| Burkholderia mallei | Fungi |
| Yersinia pestis | Aspergillus sp. |
| Francisella tularensis | Mucorales sp |
| Hemophilus influenzae | Candida sp. |
| Bordetella pertussis | Histoplasma capsulatum |
| Neisseria meningitidis | Blastomyces dermatitidis |
| Legionella pneumophila | Cryptococcus neoformans |
| Legionella-like bacteria | Coccidioides immitis |
| Bacteroides melaninogenicus | Paracoccidioides brasiliensis |
| Fusobacterium nucleatum | Pneumocystis carinii |
| Peptostreptococcus sp. | Parasites-Protozoa |
| Peptococcus sp. | Plasmodium falciparum |
| Actinomyces sp. | Entamoeba histolytica |
| Mycobacterium tuberculosis | Toxoplasma gondii |
| Other Mycobacterium sp. | Leishmania donovani |
| Mycoplasma pneumoniae | Parasites-Nematodes |
| Branhamella catarrhalis | Ascaris lumbricoides |
| Chlamydia trachomatis | Toxocara sp. |
| Chlamydia psittaci | Ancyclostoma duodenale |
| Chlamydia pneumoniae | Parasites-Cestodes |
| Coxiella burnetii (Q-fever) | Echinococcus granulosus |

Picornaviruses, especially rhinoviruses and certain echoviruses and coxsackieviruses, cause the common cold, defined as an acute, usually afebrile, viral infection of the respiratory tract, with inflammation in any or all airways, including the nose, paranasal sinuses, throat, larynx, and sometimes the trachea and bronchi.

Immunity is specific for viruses by serotype or strain, and thus immunity against one strain is not protective against subsequent infection with another strain. Although effective experimental vaccines have been developed for some rhinoviruses, adenoviruses, and paramyxoviruses, no commercial vaccine is yet available. Prophylactic interferon offers promise in patients at risk for morbidity from colds due to other complications, such as asthma or bronchitis. Interferon-alpha given intranasally limits acquisition of rhinovirus or coronavirus infection and reduces viral shedding; but may cause nasal inflammation with bleeding after prolonged exposure.

Influenza viruses (orthomyxoviruses) cause influenza, defined as an acute viral respiratory infection with influenza, a virus causing fever, coryza, cough, headache, malaise, and inflamed respiratory mucous membranes. Influenza produces widespread sporadic respiratory illness during fall and winter every year in temperate climates, often in focused single serotype epidemics, most often caused by influenza A (H3N2) viruses. Influenza B viruses typically cause mild respiratory disease but can cause significant morbidity and mortality during an epidemic.

Exposure to influenza virus by natural infection or by immunization results temporarily in resistance to reinfection with the same virus type. Vaccines that include the prevalent strains of influenza viruses reduce the incidence of infection among vaccinees when the HA and/or NA of the immunizing and infecting strains match. Anti-infective agents for influenza A types include amantadine and rimantadine, at 100 mg po bid. Amantadine and rimantadine may cause nervousness, insomnia, or other CNS side-effects, and drug resistance frequently occurs.

Severe acute respiratory syndrome (SARS) has been recently shown to be associated with a new coronavirus, SARS-CoV. Although strong evidence supports that this new coronavirus is the etiologic agent of SARS, it is possible that other pathogens might have a role in some cases of SARS.

The Centers for Disease Control and Prevention currently recommends that patients with SARS receive the same treatment that would be used for any patient with serious community-acquired atypical pneumonia. At present, the most efficacious treatment regimen, if any, is unknown. In several locations, therapy has included antivirals, such as oseltamivir or ribavirin. Steroids also have been given orally or intravenously to patients in combination with ribavirin and other antimicrobials. In the absence of controlled clinical trials, however, the efficacy of these regimens remains unknown. Early information from laboratory experiments suggests that ribavirin does not inhibit virus growth or cell-to-cell spread of one isolate of the new coronavirus that was tested. Additional laboratory testing of ribavirin and other antiviral drugs is being done to see if an effective treatment can be found.

The parainfluenza viruses are paramyxoviruses types 1, 2, 3, and 4 are closely related viruses causing many respiratory illnesses varying from the common cold to influenza-like pneumonia, with febrile croup as the most common severe manifestation.

Adenoviruses are a group of many viruses, some of which cause acute febrile disorders characterized by inflammation of the respiratory and ocular mucous membranes and hyperplasia of submucous and regional lymphoid tissue. Acute febrile respiratory disease is the usual manifestation of symptomatic adenoviral infection in children. A syndrome designated acute respiratory disease (ARD) has been observed in military recruits during periods of troop mobilization.

Vaccines containing live adenovirus types 4 and 7 have markedly reduced ARD in military populations; however, they are neither recommended nor available for civilian use. Vaccines for a few other serotypes have been developed but are not commercially available.

A special category of subjects, specifically lung transplant recipients are subject to many additional infectious agents. Cytomegalovirus is the most common viral infection, and a major cause of morbidity. Adenovirus infections have been reported, manifesting as an acute bronchitis/bronchiolitis to diffuse alveolar damage. Epstein Barr virus produces varied manifestations ranging from mononucleosis-like syndrome to posttransplant lymphoproliferative disorder. Pneumocystis carinii pneumonia often occurs due to depressed cellular immunity. Other miscellaneous infections include Pseudallerscheria boydii that mimics aspergillosis; nocardia, with manifestations including bronchopneumonia, abscess formation, cavitation, and empyema; Legionella pneumonia; and Toxoplasma gondii.

Other Exemplary Lung Disorders

Asthma is a chronic inflammatory disease of the small airways in which the airways become blocked or narrowed. These effects are usually temporary and reversible, but they cause shortness of breath, breathing trouble, and other symptoms. An asthma episode is triggered by elements in the environment. These triggers vary from person to person, but common ones include cold air; exercise; allergens such as dust mites, mold, pollen, animal dander or cockroach debris; and some types of viral infections.

When the airways come into contact with an asthma trigger, the tissue inside the bronchi and bronchioles becomes inflamed. At the same time, the muscles on the outside of the airways constrict, causing them to narrow. A thick fluid (mucus) enters the airways, which become swollen. The breathing passages are narrowed still more, and breathing is hampered.

Asthma pathogenesis favors a role of Th2 cells and eosinophils. Characteristics of asthma include mononuclear, eosinophil and mast cell infiltration of the submucosa and submucosal remodeling, including fibrosis and neovascularization. Viral upper respiratory infections have been associated with 80% of asthma exacerbations in children and 50% of all asthma episodes in adults. Human Rhinovirus has been implicated as the most common virus associated with asthma episodes. Although a controversial topic, viruses may play a role in the development of asthma. Generally, disease exacerbations arise from stimuli that are allergenic.

Chemokines, especially eotaxin and the monocyte chemoattractant proteins, are potent eosinophil chemoattractants and histamine releasing factors, making them particularly important in generating an allergic inflammation. In fact, these chemokines may be the main histamine-releasing factors in the absence of antigen and IgE antibody. Th2 cells regulate the production of IgE, and the growth and differentiation of mast cells, basophils, and eosinophils, the primary players in the allergic response.

Current treatment includes bronchodilators, anti-inflammatory medications (including anti-leukotrienes) and, recently, an anti-IgE treatment. Bronchodilators provide relief from asthma by relaxing the muscles in the air tubes. Anti-inflammatory medications work to keep the air tubes open to prevent an asthma attack. The allergen bound to IgE activates mast cells and basophils that release the chemical mediators (histamines, leukotrienes and prostaglandins) that produce the allergic response. Use of an anti-IgE antibody to bind and thus sequester IgE helps reduce the allergic response by preventing the IgE from binding to mast cells and basophils.

Chronic obstructive pulmonary disease (COPD) is an umbrella term used to describe airflow obstruction that is associated mainly with emphysema and chronic bronchitis. Emphysema causes irreversible lung damage by weakening and breaking the air sacs within the lungs. Elasticity of the lung tissue is lost, causing airways to collapse and obstruction of airflow to occur. Chronic bronchitis is an inflammatory disease that begins in the smaller airways within the lungs and gradually advances to larger airways. It increases mucus in the airways and increases bacterial infections in the bronchial tubes, which, in turn, impedes airflow.

COPD decreases the ability of the lung to take in oxygen and remove carbon dioxide. As the disease progresses, the walls of the small airways and alveoli lose their elasticity. The airway walls collapse, closing off some of the smaller air passages and narrowing larger ones. The passageways become clogged with mucus. Air continues to reach the alveoli when the lungs expand during inhalation; however, it is often unable to escape during exhalation because the air passages tend to collapse during exhalation, trapping the “stale” air in the lungs.

Exacerbations of COPD are a major cause of morbidity and mortality. The common etiological factors for exacerbations are bacterial infections, viral infections and pollutants. Airway obstruction in COPD patients may make these individuals more susceptible to the infections. Approximately 50% of COPD patients who have an exacerbation also have a bacterial infection. The most common bacterial infections are Haemophilus influenza and Streptococcus pneumonia. Viral infections are associated with 23-45% (more in the winter months) of patients hospitalized with an exacerbation. Bacterial infections also exist in COPD patients who are stable, but they are about twice as common in patients who have an exacerbation. It has been demonstrated that patients improve more quickly when treated with antibiotics, especially those with the most symptoms.

Long-term smoking is the most frequent cause of COPD. It accounts for 80 to 90 percent of all cases. A smoker is 10 times more likely than a non-smoker to die of COPD. The symptoms of COPD include: chronic cough, chest tightness, shortness of breath, an increased effort to breathe, increased mucus production, and frequent clearing of the throat.

The clinical development of COPD is typically described in three stages, as defined by the American Thoracic Society:

Stage 1: Lung function (as measured by FEV1 or forced expiratory volume in one second) is greater than or equal to 50 percent of predicted normal lung function. There is minimal impact on health-related quality of life. Symptoms may progress during this stage, and patients may begin to experience severe breathlessness, requiring evaluation by a pulmonologist.

Stage 2: FEV1 lung function is 35 to 49 percent of predicted normal lung function, and there is a significant impact on health-related quality of life.

Stage 3: FEV1 lung function is less than 35 percent of predicted normal lung function, and there is a profound impact on health-related quality of life.

In addition to smoking cessation, depending upon the severity of the disease, treatments may include bronchodilators that open up air passages in the lungs, anti-inflammatory medications, antibiotics, expectorants to help loosen up and expel mucus secretions, and exercise to strengthen muscles. People with COPD may eventually require supplemental oxygen and, in the end-stages of the disease, may have to rely on mechanical respiratory assistance.

In addition, other medications may be prescribed to manage conditions associated with COPD. These may include: Diuretics, which are given as therapy to avoid excess water retention associated with right-heart failure, which may occur in some COPD patients; Digitalis (usually in the form of digoxin), which strengthens the force of the heartbeat. It is used with caution in COPD patients, especially if their blood oxygen tensions are low, since they become vulnerable to arrhythmia when taking this drug; Painkillers, cough suppressants, and sleeping pills, which should be used only with caution, because they depress breathing to some extent.

Lung transplantation is being performed in increasing numbers and may be an option for people who suffer from severe emphysema. Additionally, lung volume reduction surgery has shown promise and is being performed with increasing frequency. However, a recent study found that emphysema patients who have severe lung obstruction with either limited ability to exchange gas when breathing or damage that is evenly distributed throughout their lungs are at high risk of death from this procedure.

Enhancing Pulmonary Delivery of Therapeutic Agents

Pulmonary delivery of therapeutic agents in subjects suffering from such diseases may well be limited by the barrier presented by the polarized epithelium lining the pulmonary system. Such epithelial cells are said to be “polarized;” that is, they are capable of generating gradients between the compartments they separate due to these distinct surfaces having distinct transport and permeability characteristics. (for reviews, see Knust, Curr. Op. Genet. Develop. 10:471-475, 2000; Matter, Curr. Op. Genet. Develop. 10:R39-R42, 2000; Yeaman et al., Physiol. Rev. 79:73-98, 1999).

Compositions adapted to provide delivery of therapeutic, diagnostic, prophylactic, or imaging molecules into and/or across polarized cells, and methods of their use for delivery of molecules into the general circulation, have been described. See, e.g., International Publication No. WO02/28408, which is hereby incorporated by reference in its entirety, including all tables, figures and claims. Generally, such methods comprise associating the therapeutic, diagnostic, prophylactic, or imaging molecules with targeting elements directed to a molecule expressed on the surface of epithelial cells that mediate transport into or across such cells. Numerous molecules are known to enter or exit biological systems by binding to a component that mediates transport of the molecule to or from the cell surface. Examples of such molecules include toxins such as diphtheria toxin, pseudomonas toxin, cholera toxin, ricin, abrin, concanavalin A; certain viruses (Rous sarcoma virus, adenovirus, etc.); transferrin; low density lipoprotein; transcobalamin (vitamin B12); hormones and growth factors such as insulin, epidermal growth factor, growth hormone, thyroid stimulating factor, calcitonin, glucagon, prolactin, lutenizing hormone, thyroid hormone, platelet derived growth factor, and VEGFs; and antibodies such as IgA, and IgM.

Particularly preferred cell surface components for use in the present invention as ligands to be targeted by a targeting moiety include, but are not limited to, receptors such as pIgR, a scavenger receptor, a GPI-linked protein, transferrin receptor, vitamin B12 receptor, FcRn, intergrins, low density lipoprotein receptor; cargo carrier fragments such as pIgR stalk, members of the PGDF, FGF, and VEGF receptor families (e.g., Flt-1, Flk-1, Flt-4, FGFR1, FGFR2, FGFR3, FGFR4), and surface antigens. This list is not meant to be limiting. Other preferred receptors include scavenger receptors (e.g., CLA-I/SR-B1, CD-36, intrinsic factor, cubilin, megalin, GP 330), p75NTR (Neurotrophin receptor), Leptin receptor, TGF-beta receptor, TGF beta receptor II, reduced folate carrier, Mannose-6-phosphate receptor, CaR (calcium receptor), A2b adenosine receptor, IGF-I receptor, IGF-II receptor, ebnerin (taste), 67 kD laminin receptor, laminin receptor precursor (LRP), TGF-beta receptor III, transcobalamin receptor, HGF-SF (hepatocyte growth factor/scatter factor, c-met) receptor, CD4 receptor, TGF-beta I receptor, c-erbB (EGF receptor), ASGP-R (asialoglycoprotein receptor), LRP (low density lipoprotein receptor related protein) receptor, CFTR (cyctic fibrosis transmembrane conductance regulator), sucrose isomaltase, receptors for toxins, viruses, and bacteria (e.g., GM1 ganglioside (cholera toxin), Galactosyl ceramide (HIV), receptor for anthrax protective antigen, CD46 (measles), 85 kD CSL receptor (cryptosporidium), GD1b (E. coli type II temperature sensitive enterotoxin (LTIIa)), GC-C Guanylyl cyclase (E. coli heat stable enterotoxin (STa)), putative Hepatitis A receptor, Toll-like receptor 5 (TLR5)), transporters/exchangers (e.g., PepT1, ENaC (sodium), GLUT-5, SGLT-1, CaT1 (calcium), EcaC (calcium), NHE 3 (Na+/H+ exchanger)), apolipoproteins (e.g., apolipoprotein A1, A2, A3, A4, A5, B, C1, C2, C3, C4, D, and/or E), aquaporin, high density lipoprotein binding proteins (e.g., ATP binding casette protein-1, scavenger receptor-BI), viral receptors (e.g., coxsakie adenovirus receptor, αv integrins, sialic acid-containing glycoproteins, CD4), and proteases (e.g., epitheliasin, Aminopeptidase N, Dipeptidylpeptidase).

Exemplary Targeting of pIgR and pIgR Fragments

A pIgR molecule has several structurally and functionally distinct regions that are defined as follows. A pIgR molecule binds polymeric immunoglobulins (IgA or IgM) on the basolateral side, and then transports the immunoglobulin to the apical side. Proteolytic cleavage of pIgR takes place on the apical side of an epithelial cell between the SC and the stalk, the former of which remains bound to and protects the immunoglobulins, and the latter of which remains bound to the apical membrane (see “Mucosal Immunoglobulins” by Mestecky et al. in: Mucosoal Immunology, edited by P. L. Ogra, M. E. Lamm, J. Bienenstock, and J. R. McGhee, Academic Press, 1999). Compounds and compositions bound to “stalks” displayed on the apical side of a cell can undergo reverse transcytosis, i.e., transcytosis in the opposite direction of forward transcytosis, i.e., from the apical side of a cell to its basolateral side. In reverse transcytosis, pIgR molecules or portions thereof move from the apical surfaces of cells that line the lumen of an organ to the basolateral surfaces of these cells. See, e.g., U.S. Pat. No. 6,072,041, which is hereby incorporated by reference in its entirety, including all tables, figures, and claims.

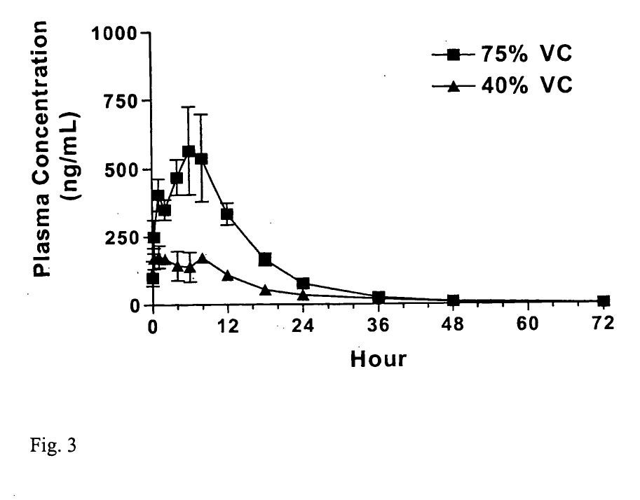

Extracellular domains 1 through 6 of pIgR molecules from several species are indicated in FIG. 3 of Piskurich et al. (J. Immunol. 154:1735-1747, 1995). In rabbit pIgR, domains 2 and 3 are encoded by a single exon that is sometimes deleted by alternative splicing. A transmembrane domain is also present in pIgR, as is an intracellular domain. The intracellular domain contains signals for transcytosis and endocytosis. Domains of a pIgR molecule that are of particular interest in the present disclosure include but are not limited to domain 5, domain 6, the B region, the stalk, the transmembrane domain, the secretory component, and the intracellular domain.

As used herein, the term “stalk” refers to a molecule having an amino acid sequence derived from a pIgR, but which does not comprise amino acid sequences derived from the secretory component. A stalk molecule comprises pIgR amino acid sequences that remain bound to the apical membrane following the apical proteolytic cleavage when such cleavage occurs, and pIgR amino acid sequences required for such cleavage. Preferred stalk molecules confer one or more transcytotic properties to a ligand bound thereto. Most preferred are stalk molecules that confer the ability to undergo apical to basolateral transcytosis to a compound or composition bound thereto.

Surprisingly, compounds or compositions bound to molecules that mediate forward transcytosis (i.e. in the basolateral to apical direction) displayed on the apical side of a cell can undergo reverse transcytosis; that is, transcytosis in the opposite direction, (i.e., from the apical side of a cell to its basolateral side). In reverse transcytosis, pIgR molecules or portions thereof move from the apical surfaces of cells that line the lumen of an organ to the basolateral surfaces of these cells. pIgR-mediated reverse transcytosis may be used to deliver agents from a lumen (e.g., the interior of the gut or the airways of the lung) to the interstitial space, circulatory system, or some other interior system, organ, tissue, portion or fluid of the body including by way of non-limiting example the lymphatic system, the vitreous humor, blood, cerebrospinal fluid, etc. A compound or composition having an element that binds to a portion of pIgR that undergoes reverse transcytosis could, due to its association with the pIgR stalk, be carried to the basolateral side of a cell, where it would be contacted with and/or released into the interstitial space, bloodstream, etc. See, e.g., U.S. Provisional Patent Application No. 60/199,423 entitled “Compositions Comprising Carriers and Transportable Complexes,” filed Apr. 23, 2000; PCT/US01/09699, entitled “Ligands Directed to the Non-Secretory Component, Non-Stalk Region of pIgR and Methods of Use Thereof,” filed Mar. 27, 2000; PCT/US01/30832 entitled “Compositions and Methods for Identifying, Characterizing, Optimizing and Using Ligands to Transcytotic Molecules,” filed Oct. 10, 2001; U.S. patent application Ser. No. 09/969,748, filed Oct. 2, 2001; U.S. Patent Application Ser. No. 60/369,548, filed Apr. 2, 2002; and U.S. Application Ser. No. 60/439,372, filed Jan. 9, 2003 (Atty Docket No. 057220-2401); each of which is hereby incorporated by reference in its entirety, including all tables, figures, and claims.

Preferred Targeting Elements

Preferred targeting elements include immunoglobulin and immunoglobulin-like polypeptides, including antibodies, single chain variable region fragments, Fabs, Fab's, etc., directed to an epithelial cell surface molecule. Wildtype antibodies have four polypeptide chains, two identical heavy chains and two identical light chains. Both types of polypeptide chains have constant regions, which do not vary or vary minimally among antibodies of the same class (i.e., IgA, IgM, etc.), and variable regions. As is explained below, variable regions are unique to a particular antibody and comprise a recognition element for an epitope.

Each light chain of an antibody is associated with one heavy chain, and the two chains are linked by a disulfide bridge formed between cysteine residues in the carboxy-terminal region of each chain, which is distal from the amino terminal region of each chain that constitutes its portion of the antigen binding domain. Antibody molecules are further stabilized by disulfide bridges between the two heavy chains in an area known as the hinge region, at locations nearer the carboxy terminus of the heavy chains than the locations where the disulfide bridges between the heavy and light chains are made. The hinge region also provides flexibility for the antigen-binding portions of an antibody.

Polyclonal antibodies are generated in an immunogenic response to a protein having many epitopes. A composition of polyclonal antibodies thus includes a variety of different antibodies directed to the same and to different epitopes within the protein. Methods for producing polyclonal antibodies are known in the art (See, e.g., Cooper et al., Section III of Chapter 11 in: Short Protocols in Molecular Biology, 2nd Ed., Ausubel et al., eds., John Wiley and Sons, New York, 1992, pages 11-37 to 11-41).

Monospecific antibodies (also known as antipeptide antibodies) are generated in a humoral response to a short (typically, 5 to 20 amino acids) immunogenic polypeptide that corresponds to a few (preferably one) isolated epitopes of the protein from which it is derived. A plurality of monospecific antibodies includes a variety of different antibodies directed to a specific portion of the protein, i.e., to an amino acid sequence that contains at least one, preferably only one, epitope. Methods for producing monospecific antibodies are known in the art (See, e.g., Cooper et al., Section III of Chapter 11 in: Short Protocols in Molecular Biology, 2nd Ed., Ausubel et al., eds., John Wiley and Sons, New York, 1992, pages 11-42 to 11-46).

A monoclonal antibody is a specific antibody that recognizes a single specific epitope of an immunogenic protein. In order to isolate a monoclonal antibody, a clonal cell line that expresses, displays and/or secretes a particular monoclonal antibody is first identified; this clonal cell line can be used in one method of producing the antibodies of the invention. Methods for the preparation of clonal cell lines and of monoclonal antibodies expressed thereby are known in the art (see, for example, Fuller et al., Section II of Chapter 11 in: Short Protocols in Molecular Biology, 2nd Ed., Ausubel et al., eds., John Wiley and Sons, New York, 1992, pages 11-22 to 11-11-36).

Variants and derivatives of antibodies include antibody and T-cell receptor fragments that retain the ability to specifically bind to antigenic determinants. Preferred fragments include Fab fragments (i.e., an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)2 (two Fab′ molecules joined by interchain disulfide bonds in the hinge regions of the heavy chains; the Fab′ molecules may be directed toward the same or different epitopes); a bispecific Fab (an Fab molecule having two antigen binding domains, each of which may be directed to a different epitope); a single chain Fab chain comprising a variable region, also known as, a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which may be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes). Derivatives of antibodies also include one or more CDR sequences of an antibody combining site. The CDR sequences may be linked together on a scaffold when two or more CDR sequences are present.

The antibodies and antibody fragments of the invention may be produced by any suitable method, for example, in vivo (in the case of polyclonal and monospecific antibodies), in cell culture (as is typically the case for monoclonal antibodies, wherein hybridoma cells expressing the desired antibody are cultured under appropriate conditions), in in vitro translation reactions, and in recombinant DNA expression systems (the latter method of producing proteins is disclosed in more detail herein in the section entitled “Methods of Producing Fusion Proteins”). Antibodies and antibody variants can be produced from a variety of animal cells, preferably from mammalian cells, with murine and human cells being particularly preferred. Antibodies that include non-naturally occurring antibody and T-cell receptor variants that retain only the desired antigen targeting capability conferred by an antigen binding site(s) of an antibody can be produced by known cell culture techniques and recombinant DNA expression systems (See, e.g., Johnson et al., Methods in Enzymol. 203:88-98, 1991; Molloy et al., Mol. Immunol. 32:73-81, 1998; Schodin et al., J. Immunol. Methods 200:69-77, 1997). Recombinant DNA expression systems are typically used in the production of antibody variants such as, e.g., bispecific antibodies and sFv molecules. Preferred recombinant DNA expression systems include those that utilize host cells and expression constructs that have been engineered to produce high levels of a particular protein. Preferred host cells and expression constructs include Escherichia coli; harboring expression constructs derived from plasmids or viruses (bacteriophage); yeast such as Saccharomyces cerevisiae or Pichia pastoris harboring episomal or chromosomally integrated expression constructs; insect cells and viruses such as Sf9 cells and baculovirus; and mammalian cells harboring episomal or chromosomally integrated (e.g., retroviral) expression constructs (for a review, see Verma et al., J. Immunol. Methods 216:165-181, 1998). Antibodies can also be produced in plants (U.S. Pat. No. 6,046,037; Ma et al., Science 268:716-719, 1995) or by phage display technology (Winter et al., Annu. Rev. Immunol. 12:433-455, 1994).

Anti-tumor Agents/Combination Therapy

Suitable agents for use in tumor therapy are described in Chabner and Longo, Cancer Chemotherapy and Biotherapy, 3^{rd} Ed., Lippincott Williams & Wilkins, 2001, which is hereby incorporated in its entirety. Preferred anti-tumor agents include small molecules commonly used in chemotherapy, such as:

alkylating agents, including nitrogen mustards, such as chlorambucil, cyclophosphamide, estramustine, ifosfamide, mechlorethamine, and melphalan; aziridine, such as thiotepa; alkyl sulfonates, such as bursulfan; nitrosureas, such as carmustine, lomustine, and streptozocin; platinum complexes, such as carboplatin and cisplatin; and nonclassic alkylators, such as altretamine, dacarbazine, procarbazine, and temozoamide; antimetabolites, including folate analogues, such as methotrexate; purine analogues, such as fludarabine, mercaptopurine, and thioguanine; adenosine analogues, such as cladribine and pentostatin; pyrimidine analogues, such as capecitabine, cytarabine, depocyt, floxuridine, fluorouracil, and gemcitabine; substituted urea, such as hydroxyurea; antitumor antibiotics, such as bleomycin, dactinomycin, daunorubicin, DaunoXome, doxorubicin, doxil, epirubicin, idarubicin, mitoxantrone, and mitomycin; epipodophyllotoxins, such as etoposide and teniposide; microtubule agents, such as docetaxel, paclitaxel, vinblastine, vincristine, and vinorelbine; camptothecin analogs, such as irinotecan and topotecan. The following list contains additional common chemotherapeutic agents:
Leucovorin calcium
Levamisole
Lomustine
Megestrol
Melphalan—L-phenylalanine mustard, L-sarcolysin
Melphalan hydrochloride
MESNA
Mechlorethamine, nitrogen mustard
Methylprednisolone
Methotrexate—Amethopterin
Mitomycin—Mitomycin-C
Mitoxantrone
Mercaptopurine
Paclitaxel Prednisone
Plicamycin—Mithramycin
Procarbazine
streptozocin—Streptozotocin
Tamoxifen
6-thioguanine
Thiotepa—triethylene thiophosphoramide
Vinblastine
Vincristine
Vinorelbine tartrate
Altretamine (Hexalen)
Asaley
AZQ (carbamic acid, diaziquone)
BCNU (carmustine)
a Bisepoxide dianhydrogalactitol
Busulfan (myleran, BSF)
Carboxyphthalatoplatinum
CBDCA (carboplatin, paraplatin)
CCNU (lomustine, CeeNu)
CHIP (iproplatin)
Chlorambucil (leukeran)
Chlorozotocin
Cis-platinum (cisplatin, platinol)
Clomesone
Cyanomorpholinodoxorubicin
Cyclodisone
Cyclophosphamide (cytoxan)
Dianhydrogalactitol
Fluorodopan
Gliadel wafer (proliferprosan 20 with carmustine implant)
E09
Estramustine phosphate sodium (emcyst)
Hepsulfam
Hexamethylmelamine
Hycanthone
Ifosfamide (IFEX)
Mechlorethamine (mechlorethamine hydrochloride, mustargen, nitrogen mustard)
Melphalan (L-PAM, alkeran)
Mesna
Methyl CCNU (semustine)
Mitomycin C
Mitozolamide Oxaliplatin
PCNU
piperazine
piperazinedione
Pipobroman
Poperazinedione
Porfiromycin
Procarbazine (matulane)
Spirohydantoin mustard
Streptozocin (zanosar)
Temodar (temozolomide)
Teroxirone
Tetraplatin
Thiophosphoramide
Thio-tepa (thioplex, TSPA, TESPA, triethylenethiophosphoramide)
Triazinate
Triethylenemelamine
Uracil nitrogen mustard
Yoshi-864

Particularly preferred anti-tumor agents are polypeptides, including interleukins, interferons, tumor necrosis factor (TNF), and therapeutic antibodies. An exemplary list of interleukins includes any of IL-1, IL-2, IL-3, IL-4, IL-5, IL-6, IL-7, IL-9, IL-10, IL-12, IL-13, IL-15, IL-18, IL-21, and functional derivatives thereof. An exemplary list of interferons includes interferon α, interferon β, interferon γ, and functional derivatives thereof.

Additional preferred anti-tumor agents include enzymes. Preferred enzymatic anti-tumor methods involve Antibody-Directed Enzyme Prodrug Therapy (ADEPT). The antibodies (or fragments thereof) direct a composition comprising an enzyme to a tumor site, and the associated enzyme converts a prodrug into an active drug at the site. Thus, the strategy is to introduce an enzyme at, near, or into tumor cells that converts an otherwise non-toxic pro-drug into a toxic substance, thereby killing tumor or cancer cells at the targeted site.

For example, thymidine kinase phosphorylates the compound gancicivir, causing it to inhibit the synthesis of DNA, resulting in cell death. This enzyme can be contained in the composition and attached to an appropriate targeting element. Gancicivir is then given systemically. Another example is cytosine deaminase, which is found in E. coli and converts 5-flurocytosine into the toxic chemotherapeutic agent, 5-flurouracil. Thus, large amounts of 5-fluorocytosine can be administered to the subject without causing harm to the normal body cells, while delivering a toxic dose specifically to cancer cells. The present methods have the additional advantage of killing tumor and cancer cells by “bystander effect,” that is, not every cell in the tumor needs to be targeted by the composition in order to eradicate the tumor completely. Thus, once a tumor cell has been killed, the cytotoxic drug can diffuse into neighboring cells and kill them as well. The successful targeting of as few as 10% of cells can lead to a 100% destruction of a tumor.

In another example, a drug useful for treating breast cancer is capecitabine, which is converted by the enzyme thymidine phosphorylase to 5-fluorouracil (5-FU). Thus, thymidine phosphorylase can be attached to the targeting elements of the compositions, and targeting elements included on the composition that bind to the tumor site. The patient is treated with capecitabine, thus delivering 5-FU to the tumor site. This embodiment can be combined with co-administration of other drugs (e.g., taxotere) that may cause specific types of cancers (e.g., breast cancers) to increase production of thymidine phosphorylase, thus enhancing the therapeutic effect.

In still further embodiments nitro eductase, thymidine kinase and adenosine deaminase can be used to convert pro-drugs such as CB1954, ganciclovir and 5-FC into cytotoxic drugs.

Additional antitumor agents for use in the present invention are nucleic acids, including but not limited to double-stranded RNA designed to provide gene silencing of tumor-associated nucleic acid(s) by RNA interference (“RNAi”) (see, e.g., Paddison et al., Proc. Nat'l Acad. Sci. USA 99: 1443-8 (2002); and Hutvagner and Zamore, Curr. Opin. Genet. Dev. 12: 225-32 (2002)); antisense nucleic acids designed to inhibit expression of tumor-associated nucleic acid(s) (see, Bavisotto, J. Exp. Med. 174: 1097-1101 (1991); gene therapy constructs designed to disrupt tumor-associated nucleic acid(s) (“knockout” constructs); gene therapy constructs designed to overexpress therapeutic nucleic acid(s); or a combination of any of these compositions.

Anti-infective Agents/Combination Therapy

Particularly preferred anti-infective agents for use in preparing invention compounds are polypeptides, including interleukins, interferons, tumor necrosis factor (TNF), and therapeutic antibodies. An exemplary list of interleukins includes any of IL-1, IL-2, IL-3, IL-4, IL-5, IL-6, IL-7, IL-9, IL-10, IL-12, IL-13, IL-15, IL-18, IL-21, and functional derivatives thereof. An exemplary list of interferons includes interferon α, interferon β, interferon γ, and functional derivatives thereof. As discussed herein, the invention compounds may be used in combination therapy with known anti-infective agents that are effective against various bacterial, viral, fungal, and parasitic infectious agents. Such agents are well described and identified in the art.

The following listings provide exemplary classes and types of anti-infective agents. One of skill in the art could readily determine appropriate strategies for combination therapies against specific infectious agents.

Anti-Bacterial Agents:

b-lactam antibiotics; including penicillins, penicillin G-like drugs (penicillin G, penicillin V, procaine penicillin, benzathine penicillin)
Penicillinase—resistant penicillins
Cloxacillin
Dicloxacillin
Methicillin
Nafcillin
Oxacillin
Ampicillin-like drugs; including ampicillin, ampicillin plus sulbactam, amoxicillin, amoxicillin
plus clavulanate
Bacampicillin
Broad-spectrum (antipseudomonal) penicillins
Azlocillin
Carbenicillin
Mezlocillin
Piperacillin
Piperacillin plus tazobactam
Ticarcillin
Ticarcillin plus clavulanate
Cephalosporins
Imipenem and meropenem
Aztreonam
Clavulanic acid, sulbactam, and tazobactam
Aminoglycosides
Amikacin
Gentamicin
Kanamycin
Neomycin
Netilmicin
Streptomycin
Tobramycin
Macrolides, Lincomycin, And Clindamycin (azithromycin, clarithromycin, clindamycin)
Erythromycin
Lincomycin
Tetracyclines
Demeclocycline
Doxycycline
Minocycline
Oxytetracycline
Tetracycline
Chloroamphenicol.
Vancomycin
Quinupristin/Dalfopristin
Metronidazole
Rifampin
Spectinomycin
Nitrofurantoin
Quinolones
Cinoxacin
Nalidixic acid
Fluoroquinolones
Ciprofloxacin
Enoxacin
Grepafloxacin
Levofloxacin
Lomefloxacin
Norfloxacin
Ofloxacin
Sparfloxacin
Trovafloxacin
Bacitracin
Colistin
Polymyxin B
Sulfonamides
   Anti-Viral Agents:
Idoxuridine (IDU)
Vidarabine (adenine arabinoside, ara-A)
Trifluridine (triflurothymidine)
Acyclovir
Famciclovir
Penciclovir
Ralacyclovir
Ganciclovir
Foscarnet
Ribavirin
Amantadine
Rimantadine
Cidoforvir
Antisense Oligonucleotides
Immune globulins
Zidovudine (ZDV, AZT)
Didanosine (ddI)
Zalcitrabine (ddC)
Stavudine (d4T)
Lamivudine (3TC)
Reverse transcriptase inhibitors (nevirapine, delavirdine)
Viral protease inhibitors

Coupling of Components

In preferred embodiments, the compounds and compositions of the present invention comprise a first element (e.g., a therapeutic agent) “coupled” in some sense to a second (or third, or fourth, etc.) element (e.g., a targeting element). The skilled artisan will understand that such moieties may be simply two portions of a single molecule (an example of two such regions may be an Fc region and an Fab region on an antibody), or two molecules linked by a tethering “linker moiety.” Numerous methods are available to the skilled artisan to provide such “coupled” molecules. Alternatively, portions may be coupled without the use of a traditional linker, e.g. chemically, or within a single open reading frame.

For example, any two components (e.g., two components independently selected from the group consisting of a polypeptide, an antibody, an antibody fragment, a single-chain variable region fragment, a small molecule, an oligonucleotide, an oligosaccharide, a polysaccharide, a cyclic polypeptide, a peptidomimetic, and an aptamer, a poly(ethylene oxide), a dextran, etc.) may be chemically cross-linked by a linker having chemistry compatible with a site on each component. Crosslinkers are well known to those of skill in the art, and may be obtained commercially (see, e.g., Pierce Chemical Company Catalog and Handbook 1994-95, pages O-90 through O-110, which is hereby incorporated by reference) or synthesized as needed.

Alternatively, in cases where both components are peptides, the components may be coupled “genetically”; that is, the first and second elements may be expressed as a chimeric protein or fusion protein. For example, U.S. Pat. No. 6,072,041 to Davis et al. is drawn to fusion proteins that are directed to the secretory component of pIgR. Ferkol et al., Am. J. Respir. Crit. Care Med. 161:944-951, 2000, discloses a fusion protein consisting of a single-chain variable region fragment directed to the secretory component (SC) of human pIgR and a human alpha (1)-antitrypsin. U.S. Pat. No. 6,042,833 to Mostov et al. discloses “genetic fusions” and “fusion proteins” that include ricin A, poly-(L)-Lys, or a phage surface protein.

In a similar manner, molecular biology may be used to introduce domains into a component that can combine with a complementary domain on a second component. For example, a coiled-coil domain sequence may be attached to a first targeting element and a second targeting element to provide the complementarity necessary to achieve binding between the two elements. Alternatively, cysteine residues may be introduced into the two targeting elements for the formation of a disulfide-bonded complex.

In an alternative approach, the various components of the compositions described herein can be associated with a particle or capsule. Methods for producing particulate administration systems for delivery of biologically-relevant molecules are well known to those of skill in the art. Such particles are preferably porous and/or biodegradable so that molecules (e.g., drugs, vaccines, vitamins, polypeptides, antibodies, etc.) contained within the particle may be released once delivered into the circulation; however, nonporous and/or nonbiodegradable particles (e.g., liposomes) are also known to those of skill in the art. Preferred particles and capsules, including microparticles, nanoparticles, microcapsules, and nanocapsules are disclosed in, e.g., U.S. Pat. No. 5,702,727; U.S. Pat. No. 5,620,708; U.S. Pat. No. 5,607,691; U.S. Pat. No. 4,610,896; U.S. Pat. No. 5,149,794; U.S. Pat. No. 6,197,349; U.S. Pat. No. 6,159,502; U.S. Pat. No. 5,785,976; Chiu et al., Biomaterials 23: 1103-12 (2602); Andrianov et al., Biomaterials 19: 109-115 (1998); Soppimath et al., J. Controlled Release 70: 1-20 (2001); McPhail et al., Intl. J. Pharmaceutics 200: 73-86 (2000); Müller et al., Eur. J. Pharmaceut. Biopharmaceut. 50: 161-177 (2000); Franssen et al., J. Controlled Release 60: 211-21 (1999); Prokop et al., Biotechnol. and Bioeng. 75: 228-232 (2001); Allémann et al., Adv. Drug Deliv. Rev. 34: 171-89 (1998); Vinogradov et al., Adv. Drug Deliv. Rev. 54: 135-47 (2002); Jung et al., Eur. J. Pharmaceut. Biopharmaceut. 50: 147-60 (2000); Martin et al., Biomaterials 19: 69-76 (1998); Vervoort et al., Intl. J. Pharmaceutics 172: 137-45 (1998); J. Controlled Release 65: 49-54 (2000); Davda and Labhasetwar, Intl. J. Pharmaceutics 223: 51-9 (2002); Düzgünes and Nir, Adv. Drug Deliv. Rev. 40: 3-18 (1999); Nagayasu et al., Adv. Drug Deliv. Rev. 40: 75-87 (1999); Leroueil-Le Verger et al., Eur. J. Pharmaceut. Biopharmaceut. 46: 137-143 (1998); Breton et al., Biomaterials 19: 271-81 (1998); Konan et al., Intl. J. Pharmaceutics 233: 239-52 (2002); Duncan et al, Eur. Polymer J. 37: 1821-6 (2001); and Stenekes et al., Biomaterials 22: 1891-8 (2001), each of which is hereby incorporated by reference in its entirety.

Pharmaceutical Compositions

The compositions of the present invention provide for delivery of therapeutic agents to a subject in need thereof. The compositions of the invention can further comprise other chemical components, such as diluents and excipients. A “diluent” is a chemical compound diluted in a solvent, preferably an aqueous solvent, that facilitates dissolution of the therapeutic agent in the solvent, and it may also serve to stabilize the biologically active form of the targeting element or one or more of its components. Salts dissolved in buffered solutions are utilized as diluents in the art. For example, preferred diluents are buffered solutions containing one or more different salts. A preferred buffered solution is phosphate buffered saline (particularly in conjunction with compositions intended for pharmaceutical administration), as it mimics the salt conditions of human blood. Since buffer salts can control the pH of a solution at low concentrations, a buffered diluent rarely modifies the biological activity of a biologically active peptide.

An “excipient” is any more or less inert substance that can be added to a composition in order to confer a suitable property, for example, a suitable consistency or to form a drug. Suitable excipients and carriers include, in particular, fillers such as sugars, including lactose, sucrose, mannitol, or sorbitol cellulose preparations such as, for example, maize starch, wheat starch, rice starch, agar, pectin, xanthan gum, guar gum, locust bean gum, hyaluronic acid, casein potato starch, gelatin, gum tragacanth, polyacrylate, methyl cellulose, hydroxypropylmethyl-cellulose, sodium carboxymethylcellulose, and/or polyvinylpyrrolidone (PVP). If desired, disintegrating agents can also be included, such as cross-linked polyvinyl pyrrolidone, agar, or alginic acid or a salt thereof such as sodium alginate. Other suitable excipients and carriers include hydrogels, gellable hydrocolloids, and chitosan. Chitosan microspheres and microcapsules can be used as carriers. See WO 98/52547 (which describes microsphere formulations for targeting compounds to the stomach, the formulations comprising an inner core (optionally including a gelled hydrocolloid) containing one or more active ingredients, a membrane comprised of a water insoluble polymer (e.g., ethylcellulose) to control the release rate of the active ingredient(s), and an outer layer comprised of a bioadhesive cationic polymer, for example, a cationic polysaccharide, a cationic protein, and/or a synthetic cationic polymer; U.S. Pat. No. 4,895,724. Typically, chitosan is cross-linked using a suitable agent, for example, glutaraldehyde, glyoxal, epichlorohydrin, and succinaldehyde. Compositions employing chitosan as a carrier can be formulated into a variety of dosage forms, including pills, tablets, microparticles, and microspheres, including those providing for controlled release of the active ingredient(s). Other suitable bioadhesive cationic polymers include acidic gelatin, polygalactosamine, polyamino acids such as polylysine, polyhistidine, polyomithine, polyquaternary compounds, prolamine, polyimine, diethylaminoetliyldextran (DEAE), DEAE-imine, DEAE-methacrylate, DEAE-acrylamide, DEAE-dextran, DEAE-cellulose, poly-p-aminostyrene, polyoxethane, copolymethacrylates, polyamidoamines, cationic starches, polyvinylpyridine, and polythiodiethylaminomethylethylene.

The compositions of the invention can be formulated in any suitable manner. Suitable formulations include dry particulate and liquid formulations. Dry formulations include freeze dried and lyophilized powders, which are particularly well suited for aerosol delivery to the sinuses or lung, or for long term storage followed by reconstitution in a suitable diluent prior to administration. The particular amount of biologically active component to be delivered will depend on many factors, including the effect to be achieved, the type of organism to which the composition is delivered, delivery route, dosage regimen, and the age, health, and sex of the organism. As such, the particular dosage is left to the ordinarily skilled artisan's discretion. Additionally, particle size may be controlled to achieve optimal delivery to a specific region of the organ (e.g., the lung). Preferred particle sizes are between about 1 μm and about 20 μm, preferably between about 1 μm and about 10 μm, even more preferably between about 2 μm and about 7 μm, and most preferably between about 3 μm and about 5 μm. The term “about” in this context refers to +/− 10% of a given measurement.

It will be readily apparent to those skilled in the relevant arts that other suitable modifications and adaptations to the methods and applications described herein may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

### Administration

The compounds administered according to the invention can be administered according to various methods, such as instillation, inhalation, exposure to the nasal and/or oral membranes (e.g., sniffing or nasal drops), intravenous administration, or intraperitoneal administration, depending on the particular application. Instillation and inhalation are especially effective methods of administration. The composition can also be nebulized, aerosolized, atomized, or made as a mist, and administered through inhalation or instillation. The most desirable mode of administration will be determined in any particular application, but the most preferable mode of administration in inhalation of the compound, so that administration can occur without surgical intervention or the presence of medical personnel, and the methods can be self-administered by the subject.

### EXAMPLE 2

### Multimeric sFvs

In vitro genetic manipulation has been used to alter the reading frame of sFvs so as to create derivatives that have substitutions or insertions of amino acids with reactive sites. See, e.g., U.S. patent application Ser. No. 09/969,748, Example 6, and International Publication No. WO02/28408, Example 6, each of which is hereby incorporated by reference in this regard. The two variable regions of a sFv that combine to form a ligand binding site are known as V(H) and V(L). In a monomeric sFvs, the V(H) and V(L) of each molecule are associated with each other. In one type of dimeric sFv, the V(H) of one monomer [V(H)1] is associated with the V(L) of another monomer [V(L)2], and vice versa [i.e., V(H)2 is associated with V(L)1].

The length and composition of the linker between the V(H) and V(L) regions in an sFv is one factor that influences the tendency of an sFv to form monomers or multimers (Todorovska et al., Design and application of diabodies, triabodies and tetrabodies for cancer targeting, J. Immunol. Meth. 248:47-66 (2001); Arndt et al., Biochemistry 37 12918-12926 (1993). For example, a sFv molecule in which there is a relatively short linker between the V(H) and V(L) regions may be less likely to fold back upon itself and form a monomer. Thus, “short linker” sFv derivatives are often more likely to form dimers, as their V(H) and V(L) regions must pair with, respectively, the V(L) and V(H) regions of a second sFv molecule. Often, sFv derivatives with relatively long linkers between the V(H) and V(L) regions may fold back upon themselves, and therefore may have a greater tendency to form monomers. However, some sFv derivatives with long linkers between V(H) and V(L) may have some tendency to form multimers.

Various amino acid sequences are known that may serve as suitable spacers in the compounds of the invention (for a review, see Simons, Spacers, probability, and yields, Bioconjug Chem 1999 Jan.-Feb.; 10(1):3-8). Some non-limiting examples of sequences that have been used in sFvs include EGKSSGSGSESKEF (SEQ ID NO: 10), one or more copies of GGGGS [also known as (G₄S)ₓ] (Newton et al., Angiogenin single-chain immunofusions: influence of peptide linkers and spacers between fusion protein domains, Biochemistry 1996 Jan. 16;35(2):545-53), GSGS [also known as (GSGS)ₓ] and GSSG [also known as (GSSG)ₓ].

APL10 is an exemplary sFv coding sequence. To facilitate affinity purification, protein A interacts with the VH chain of APL10.

### EXAMPLE 3

### IL-2-sFv Conjugates

Human IL-2 is synthesized as a precursor protein of 153 amino acids, which includes a 20 amino acid hydrophobic leader sequence. The IL-2 molecule has a molecular weight of about 15.4 kD and a slightly basic pI. The protein comprises a single intramolecular disulfide bond (Cys58-Cys105) that is necessary for the biological activity of IL-2 (Yamada et al., Importance of disulfide linkage for constructing the biologically active human interleukin-2, Arch Biochem Biophys 257:194-199, 1987).

Some forms of IL-2 comprise chemical modifications. It has been reported that O-glycosylation occurs at Thr3 of bovine IL-2, and that variants with different masses due to glycosylation exist. However, non-glycosylated IL-2 remains biologically active (Kuhnle et al., Bovine interleukins 2 and 4 expressed in recombinant bovine herpesvirus 1 are biologically active secreted glycoproteins, J Gen Virol 77(Pt 9):2231-2240, 1996).

Recombinant human IL-2, expressed in either E. coli or COS cells, has been shown to be phosphorylated by protein kinase C in vitro (Kung et al., Phosphorylation of human interleukin-2 (IL-2), C Mol Cell Biochem 89:29-35, 1989). The phosphorylated tryptic peptide was identified as the N-terminal fragment containing a single phosphorylation site at the serine residue at position 7 (Ser7). There was no difference in biological activity between non-phosphorylated and phosphorylated IL-2, as determined by a T cell growth assay.

In order to generate and isolate mRNAs encoding IL-2, peripheral blood mononuclear cells (PBMC) were prepared and transferred into plates the wells of which had been precoated with mouse anti-human CD3 monoclonal antibody (BD PharMingen, San Diego, Calif.). The plates had been treated with 10 μg/ml of anti-CD3 and washed 3 times before cells were added to the wells; commercially available plates that have been coated with anti-CD3 before sale may also be used (BD BioCoat T-cell Activation Plates, BD PharMingen). Mouse anti-human CD28 monoclonal antibody (BD PharMingen) was then added to 1 ug/ml, and the plates were incubated at 37° C. for 6 hours.

Total cellular RNA was extracted from the stimulated cells using Trizol (LifeTechnologies, Gaithersburg, Md.) essentially according to the manufacturer's instructions. Single strand cDNA copies of the IL-2 message were generated using oligo(dT) primers and the ThermoScript RT-PCR system (Life Technologies) essentially according to the manufacturer's recommendations.

Sequences encoding IL-2 and part of the synthetic linker were amplified via the PCR with the primers “IL-2FormMut3” and “IL-2_Rev2”:

| IL-2ForMut3 (SEQ ID NO:1): | | 5′-CACCATGTACAGGATGCAACTGCTGTCTTG-3′ | | | | |
|---|---|---|---|---|---|---|
| IL-2Rev2 (SEQ ID NO:2): | | 5′-GATTTGCCGCTACCGGAAGTCGACCCAGTTAGTGTTGAGATGATGCTTTGA-3′ | | | | |
| PCR is performed at about 60° C. for 25 cycles. | | | | | | |
| The sequence of IL-2 cDNA (GENBANK accession number E00210, ATG | | | | | | |
| underlined) is as follows (SEQ ID NO: 3): | | | | | | |
| TCACTCTCTT | TAATCACTAC | TCACAGTAAC | CTCAACTCCT | GCCACAATGT | ACAGGATGCA | 60 |
| ACTCCTGTCT | TGCATTGCAC | TAAGTCTTGC | ACTTGTCACA | AACAGTGCAC | CTACTTCAAG | 120 |
| TTCTACAAAG | AAAACACAGC | TACAACTGGA | GCATTTACTG | CTGGATTTAC | AGATGATTTT | 180 |
| GAATGGAATT | AATAATTACA | AGAATCCCAA | ACTCACCAGG | ATGCTCACAT | TTAAGTTTTA | 240 |
| CATGCCCAAG | AAGGCCACAG | AACTGAAACA | TCTTCAGTGT | CTAGAAGAAG | AACTCAAACC | 300 |
| TCTGGAGGAA | GTGCTAAATT | TAGCTCAAAG | CAAAAACTTT | CACTTAAGAC | CCAGGGACTT | 360 |
| AATCAGCAAT | ATCAACGTAA | TAGTTCTGGA | ACTAAAGGGA | TCTGAAACAA | CATTCATGTG | 420 |
| TGAATATGCT | GATGAGACAG | CAACCATTGT | AGAATTTCTG | AACAGATGGA | TTACCTTTTG | 480 |
| TCAAAGCATC | ATCTCAACAC | TAACTTGATA | ATTAAGTGCT | TCCCACTTAA | AACATATCAG | 540 |
| GCCTTCTATT | TATTTAAATA | TTTAAATTTT | ATATTTATTG | TTGAATGTAT | GGTTTGCTAC | 600 |
| CTATTGTAAC | TATTATTCTT | AATCTTAAAA | CTATAAATAT | GGATCTTTTA | TGATTCTTTT | 660 |
| TGTAAGCCCT | AGGGGCTCTA | AAATGGTTTC | ACTTATTTAT | CCCAAAATAT | TTATTATTAT | 720 |
| GTTGAATGTT | AAATATAGTA | TCTATGTAGA | TTGGTTAGTA | AAACTATTTA | ATAAATTTGA | 780 |
| TAAATATAAA | AAAA | | | | | 794 |
| The coding sequence of IL-2 cDNA is as follows (SEQ ID NO: 4): | | | | | | |
| ATGTACAGGA | TGCAACTCCT | GTCTTGCATT | GCACTAAGTC | TTGCACTTGT | CACAAACAGT | 60 |
| GCACCTACTT | CAAGTTCTAC | AAAGAAAACA | CAGCTACAAC | TGGAGCATTT | ACTGCTGGAT | 120 |
| TTACAGATGA | TTTTGAATGG | AATTAATAAT | TACAAGAATC | CCAAACTCAC | CAGGATGCTC | 180 |
| ACATTTAAGT | TTTACATGCC | CAAGAAGGCC | ACAGAACTGA | AACATCTTCA | GTGTCTAGAA | 240 |
| GAAGAACTCA | AACCTCTGGA | GGAAGTGCTA | AATTTAGCTC | AAAGCAAAAA | CTTTCACTTA | 300 |
| AGACCCAGGG | ACTTAATCAG | CAATATCAAC | GTAATAGTTC | TGGAACTAAA | GGGATCTGAA | 360 |
| ACAACATTCA | TGTGTGAATA | TGCTGATGAG | ACAGCAACCA | TTGTAGAATT | TCTGAACAGA | 420 |
| TGGATTACCT | TTTGTCAAAG | CATCATCTCA | ACACTAACTT | GA | | 462 |

While the following examples describe the preparation of IL-2-sFv conjugates as fusion proteins, the skilled artisan will understand that additional methods (e.g., chemical crosslinking, encapsulation in particles, etc.) may be employed to associate IL-2 with an appropriate targeting element.

The IL-2 PCR product was combined with an sFv-encoding PCR product using overlap PCR, a form of PCR that joins two PCR products together, as described in U.S. patent application Ser. No. 09/969,748, and International Publication No. WO02/28408, each of which is hereby incorporated by reference in this regard. In this method, the intended junction sequence is designed into the PCR primers (at their 5′ ends). Following the initial amplification of each individual polypeptide-encoding sequence, the various products are diluted and combined, denatured, annealed, and extended. An otherwise standard PCR is then performed using “final” forward and reverse primers.

The primers used for the overlap PCR were designed to include sequences encoding a synthetic linker that is connected to the sFv polypeptide. The linker includes a 13 amino acid spacer (Gly-Ser-Thr-Ser-Gly-Ser-Gly-Lys-Ser-Ser-Glu-Gly-Lys; SEQ ID NO:5) that has previously been shown to facilitate the correct folding of the fusion protein between IL-2 and a sFv directed against the alpha-folate receptor (Melani et al., Targeting of interleukin 2 to human ovarian carcinoma by fusion with a single-chain Fv of antifolate receptor antibody, Cancer Res 58(18):4146-4154, 1998). The sFv was first amplified from plasmid DNA (pSyn5AF which is the bacterial expression vector pSyn expressing the 5A sFv; see U.S. patent application Ser. No. 09/969,748, and International Publication No. WO02/2840). The primers used were as follows.

| | |
|---|---|
| sFvFor (SEQ ID NO:6): | 5′-GTAGCGGCAAATCCTCTGAAGGCAAACAGGTGCAGCTGGTGC-AATCAGGGGGA-3′ |
| sFvRev4 (SEQ ID NO:7): | 5′-ACCTAGGACGGTGACCTTGGTCCC-3′ |

This PCR was performed at about 72° C. for about 25 cycles.

The IL-2, linker, and sFv sequence was amplified from a mixture of the IL-2 and sFv PCR products using the primers described above. Three cycles of PCR were performed at about 45° C. followed by about 25 cycles performed at about 68° C.

The PCR product from the overlap PCR was gel purified and cloned directly into the mammalian expression vector pcDNA3.1D/V5-His-TOPO® expression vector (Invitrogen, Carlsbad, Calif.). This expression vector includes a CMV-derived promoter for high-level, constitutive expression; a C-terminal V5 epitope tag that can be detected with anti-V5 antibody; and a further C-terminal 6×His tag that can be detected with an anti-6×His tag antibody or used to purify the IL-2-5A fusion protein. Anti-V5 and anti-6×His antibodies are available from Invitrogen.

In the alternative, to create a bispecific ligand consisting of an sFv specific to pIgR and recombinant IL-2, a genetic fusion is constructed in the IL-2 encoding sequence (see, e.g., Christ et al., Clin. Cancer Res. 7: 1385-97 (2001) describing pcDNA3.1/huCH3-IL-2 vector) inserted between the sequences encoding the pel-B leader and the beginning of the sFv encoding sequence.

The construct may be expressed in any suitable organism that is compatible with the cloning vector, and purified protein is isolated by FPLC using a Protein-A affinity column followed by purification on an immobilized metal affinity column.

### EXAMPLE 4

### Expression of IL-2-sFv Conjugates

The DNA from Example 3 was used to transform E. coli, and transformants were selected for using ampicillin as the vector comprises an ampicillin resistance gene. Individual colonies were selected and grown in LB media containing ampicillin. Small scale preparations (mini-preps) of plasmid DNA from 8 colonies were prepared. The predicted structures of four independently selected plasmids was confirmed by digestion with XbaI and gel electrophoresis of the digested DNA. All four of the candidates showed a electrophoresis pattern consistent with the expected product. The nucleotide sequence of the chimeric reading frame that is found in the expression constructs and which encodes the IL-2-sFv fusion protein was determined in order to confirm the accuracy and fidelity of the PCR reactions.

A large scale preparation of plasmid DNA from one of the sequence-confirmed transformants was prepared and used to transiently transfect COS-1 cells using LipofectAMINE 2000 (Life Technologies, Gaithersburg, Mass.) essentially according to the manufacturer's instructions (see Whitt et al., Unit 9.4, pages 9-11 to 9-12, and Unit 16.13, Aruffo, pages 16-53 to 16-55 in: Short Protocols in Molecular Biology, 2nd Ed., Ausubel et al., editors, John Wiley and Sons, New York, 1992). Anti-sFv polyclonal antibody was used to detect fusion proteins containing the sFv polypeptide. Transfectants are also screened for production and the secretion of the IL-2-sFv fusion protein by ELISA or Western analysis using antibodies to human IL-2 (Genzyme) and antibodies to the V5 epitope. Antibodies to human IL-2 are commercially available from, e.g., Research Diagnostics, Inc. (Flanders, N.J.) and Sigma Chemical Corp. (St. Louis, Mo.). The desired fusion protein will be detected by all three of the antibodies. Supernatant from transfected cells, in some instances at least semi-purified by IMAC chromatography, was used in further experiments.

IMAC chromatography was used to purify IL-2-sFv fusion protein from transiently transfected cells. In brief, about 400 ml of media from transfected COS-1 cells incubated for 48 to 144 hours was harvested. The media was pooled and Imidazole was added to a final concentration of 10 mM. A Pellicon cassette System (Millipore Bioscience, Bedford, Mass.) was used to concentrate the pool to a final volume of ˜75 ml. The concentrated sample was then purified using a nickel column, to which the 6×His tag binds.

### EXAMPLE 5

### Preparation of Bacterial Expression Constructs Encoding IL-2-sFv Fusion Proteins

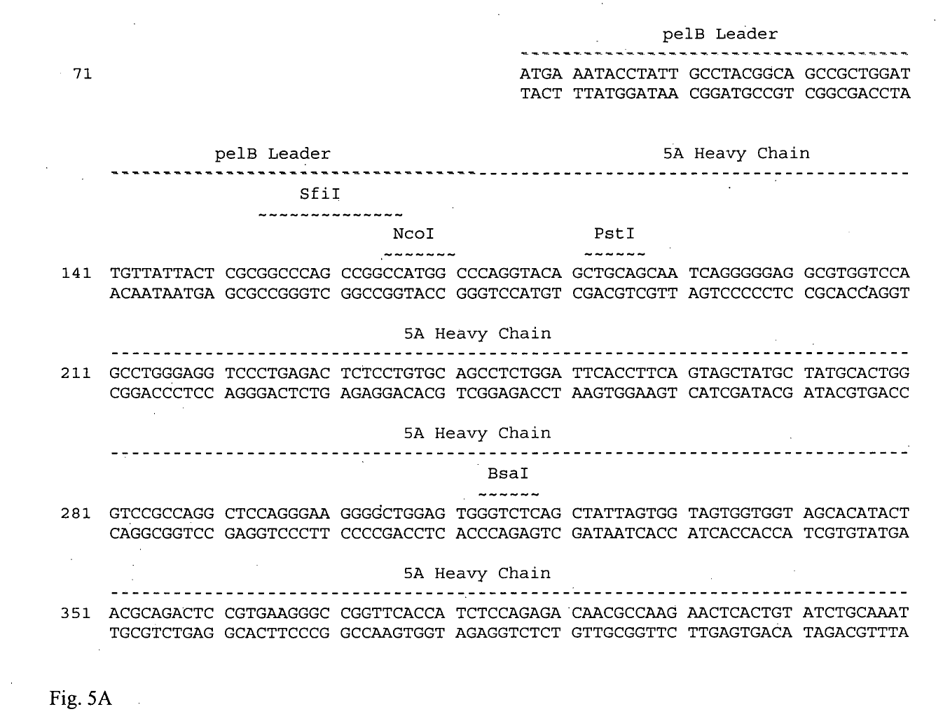

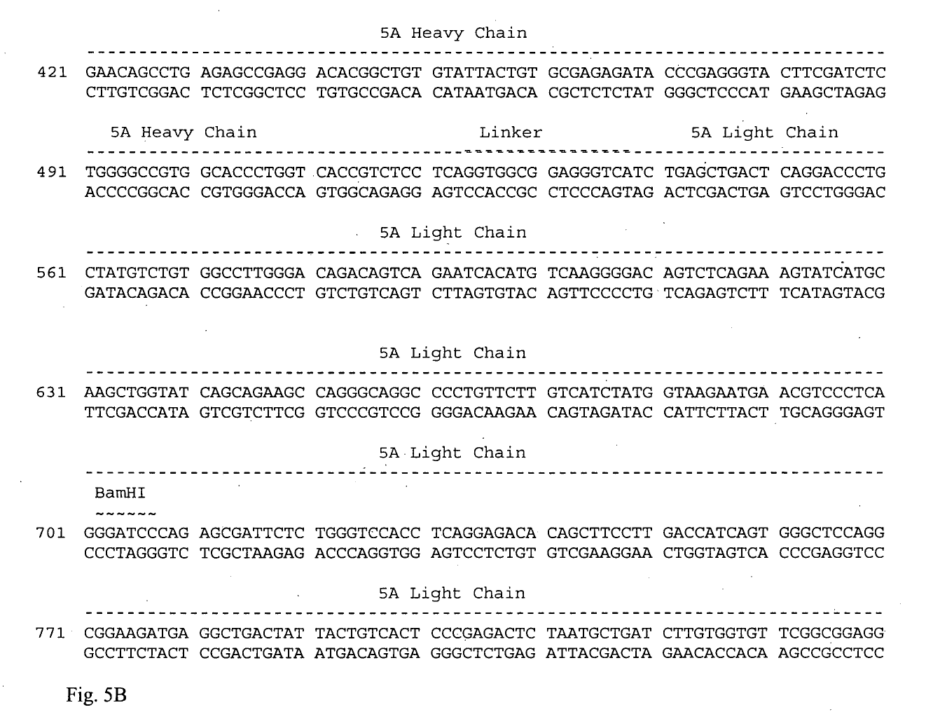

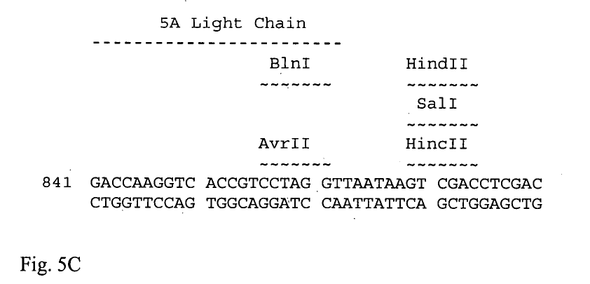

A Carboxy terminal fusion of IL-2 with a pIgR-directed sFv designed to favor dimeric sFv formation was constructed by cloning IL-2 without its signal peptide into the AvrII site of the sFv depicted in FIG. 5. A linker comprising of (Gly₃ Ser)₂ was included in the 5′ oligonucleotides and two Stop codons were included in the 3′ oligonucleotides.

The following primers were used to amplify IL-2 without its signal sequence from IL-2/5A cloned into pcDNA3.1D/V5-His-TOPO.

| | |
|---|---|
| AvrII_gggsX2_IL2_For (SEQ ID NO: 8): | 5′-GATCCCTAGGTGGCGGCGGAAGCGGCGGAGGCTCCGCACCTACTTCAAGTTCTACAAAG-3′ |
| IL2_STOP_Xhol_Rev (SEQ ID NO: 9): | 5′-CTCGAGTTATTAAGTTAGTGTTGAGATGATGCTTTGAC-3′ |

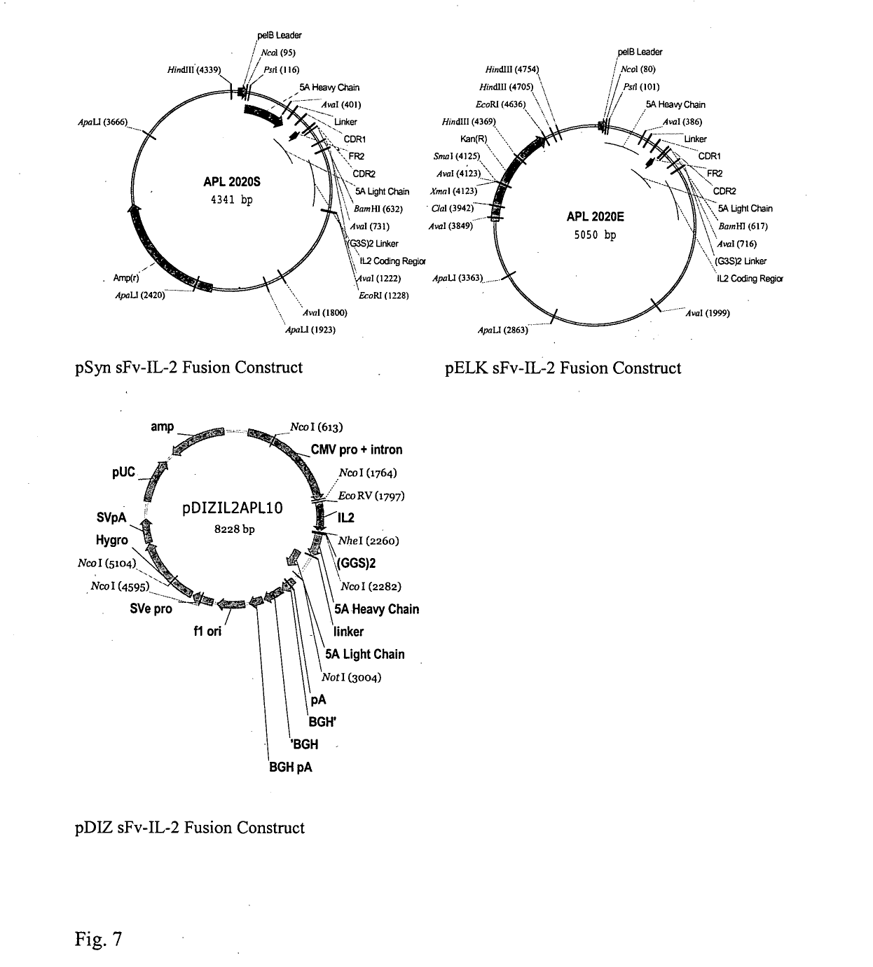

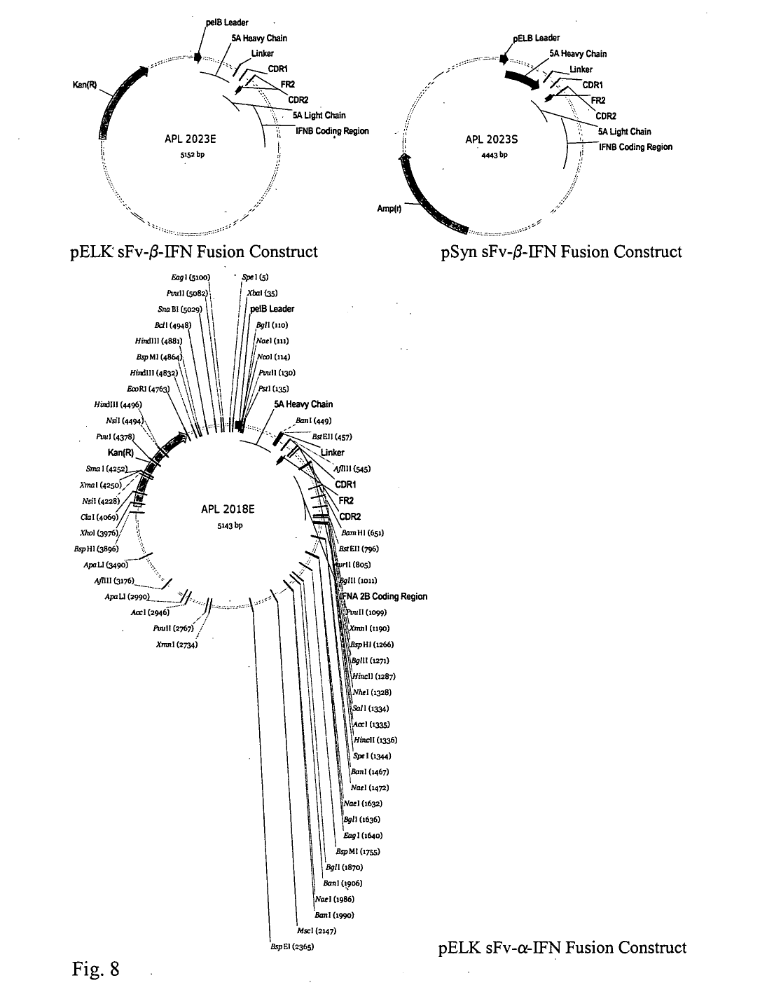

Five cycles of PCR were performed at 55° C. followed by 30 cycles performed at 60° C. The PCR product was cloned into an intermediate vector: pCR-Bluntll-TOPO (Invitrogen, Carlsbad, Calif.). The IL-2 PCR product was cut out from this intermediate vector using AvrII and EcoRI and cloned into the AvrII site of a pIgR-directed sFv in the bacterial expression vector pSyn (Griffiths et al., EMBO J. 13:3245-60, 1994). A plasmid map of the pSyn construct is provided in FIG. 7.

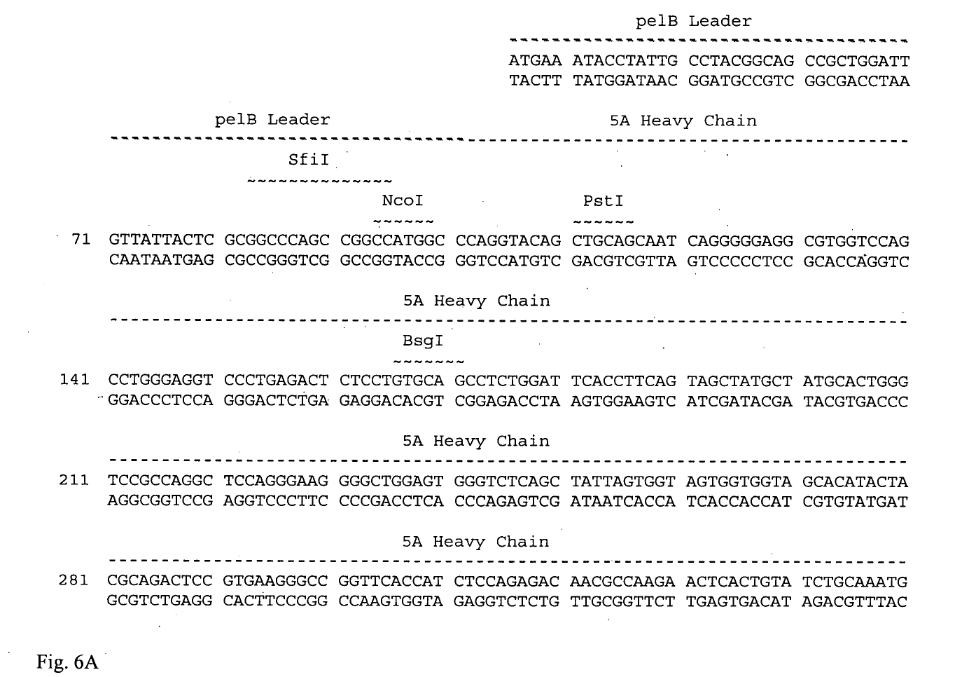

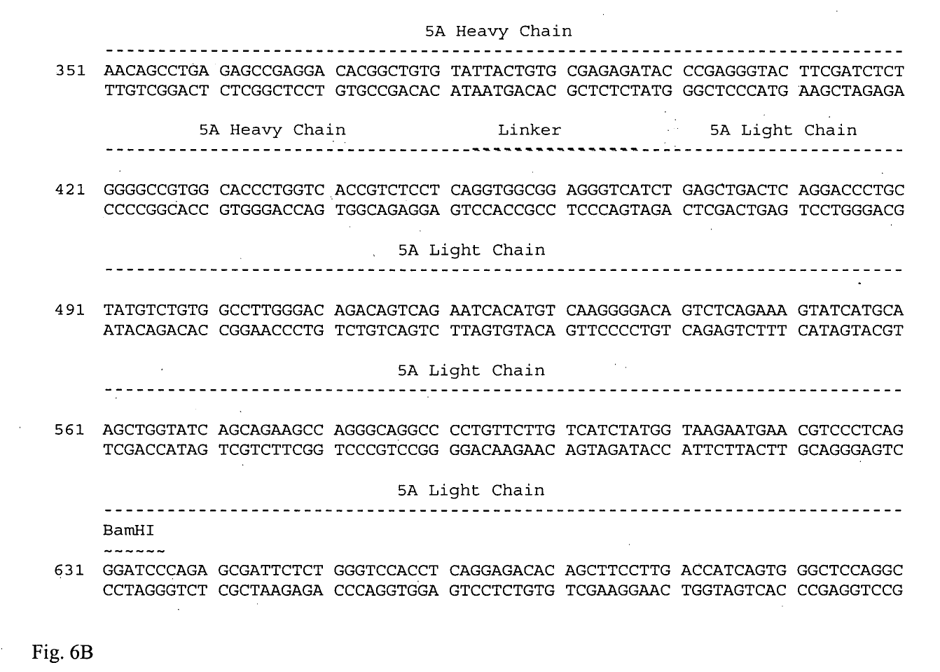

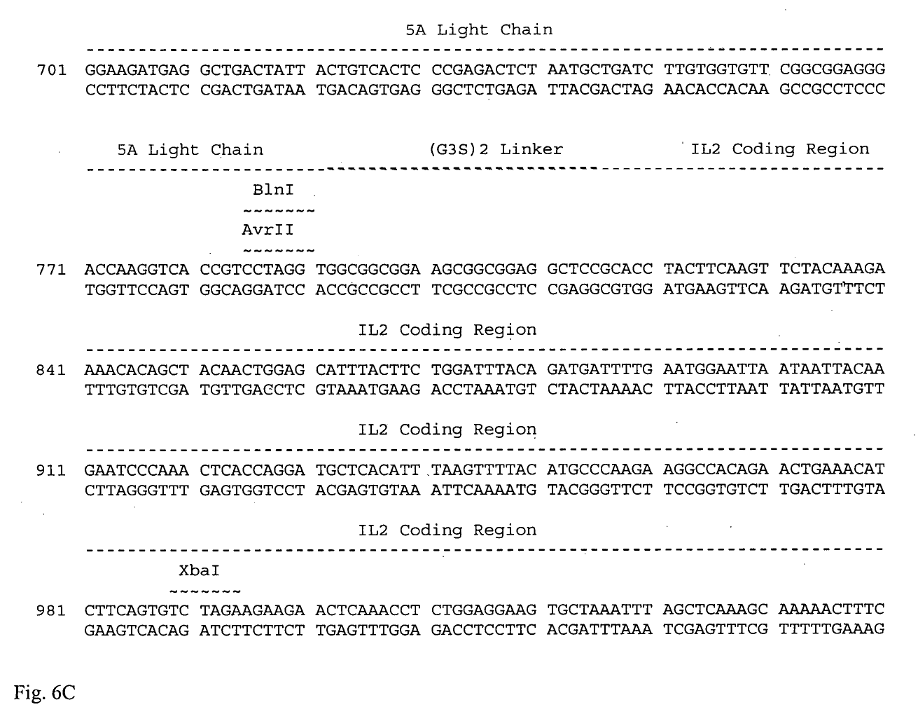

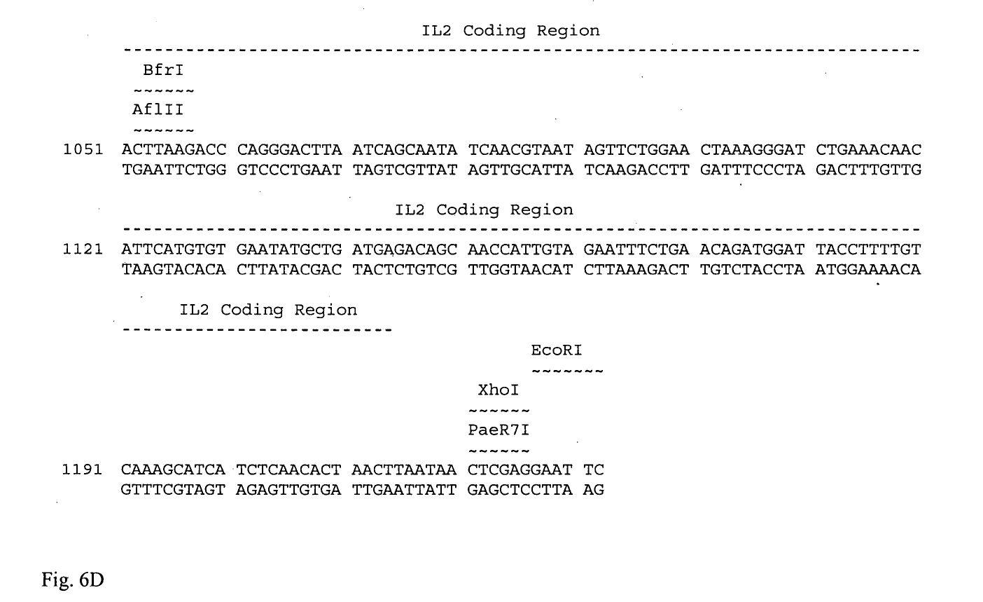

Alternatively, the IL-2 PCR product was cut out from this intermediate vector using AvrII and XhoI and cloned into the AvrII site of the sFv in the bacterial fermentation expression vector pELK (Nielsen et al., Biochim. Biophys. Acta 1591: 109-18, 2002). A plasmid map of the pELK construct is also provided in FIG. 7. The DNA was used to transform E. coli, and transformants were selected for using ampicillin as the vector comprises an amipicillin resistance gene. Individual colonies were selected and grown in LB media containing ampicillin. Small scale preparations (mini-preps) of plasmid DNA from 8 colonies were prepared. The nucleotide sequence of the chimeric reading frame that is found in the expression constructs and which encodes the IL-2-sFv fusion protein (FIG. 6) was determined in order to confirm the accuracy and fidelity of the PCR reactions.

### EXAMPLE 6

### Expression of IL-2-sFv Conjugates

A large scale preparation of plasmid DNA from one of the sequence confirmed transformants cloned into pSyn was prepared and used to transform E. coli. BL21-CodonPlus Competent cells (Stratagene). Expression of the fusion protein was induced with IPTG (De Bellis & Schwartz, 1990) and the culture was grown at 25C overnight. Fusion protein was harvested from the periplasm (Breitling et al., 1991) and loaded onto a 1 ml Protein A column for purification. Protein A interacts with the VH chain of APL10 and permits affinity purification.

Fusion protein that had been prepared by protein A affinity purification after bacterial expression, was used in transcytosis assays. Polyclonal antibody to sFv, or polyclonal antibody to the IL-2, was used to detect the APL 10-IL-2 fusion protein in both apical and basolateral media. The transcytosis was dependent on the presence of the pIgR stalk as demonstrated by the fact that transcytosis was not observed in control (non-transfected) MDCK cells.

### EXAMPLE 7

### Transwell Transcytosis Assay

This example provides an in vitro transcytotic assay that can be used in determining whether a targeting element confers apical to basolateral transcytosis to an therapeutic agent.

The transcytosis assay can be conducted using polarized cells, such as Madin-Darby Canine Kidney cells. See, e.g., Brown et al., Traffic 1: 124-40 (2000). Other appropriate cells for use in transcytosis assays include CaLu-3, Caco-2, HT29, or other appropriate cells that preferably form polarized cell layers in suitable culture systems. The cells may be transfected if necessary to express appropriate targets for binding of the ligands, particularly bispecific or multispecific ligands.

MDCK cells expressing pIgR were grown in Transwell® permeable tissue culture supports (Costar), which allows the cells to receive nutrients from the top and bottom sides of the cell monolayer. Each permeable well of a 12-well Transwell® plate was seeded with 5×10⁵ cells and grown for 3 to 5 days. When the MDCK cell layer becomes confluent, the cells are oriented with their apical membrane facing upwards. Tight junctions form between the cells to prevent paracellular movement of proteins.

IL-2-sFv fusion protein was added to the apical side (2 μg in 300 μl media) of the Transwell® cup while the basolateral chamber contained 800 μl media. The plate was placed in a 37° C. incubator for 16 h. The apical and basolateral media was transferred to microfuge tubes and the cell layers were washed three times with cold PBS (10 mM sodium phosphate pH 7.3, 150 mM NaCl), then lysed with 250 μl % NP-40 in PBS. The cell lysates were transferred to microfuge tubes and centrifuged for 5 minutes at 16,000×g to pellet the nuclei. The soluble lysates were transferred to new tubes and 100 μl of 10% Protein A-sepharose beads was added to each apical, basolateral and cell lysate tube. The tubes were placed on a rotating platform overnight at 4° C. to allow the sFv portion of the fusion protein to bind to protein A.

After washing the protein A-sepharose beads three times with PBS, 100 μl of non-reducing sample buffer was added to each tube and heated at 90° C. for 3 minutes. The samples were run on 4-15% SDS-PAGE gels and then transferred to PVDF membranes. Western blot analysis was done on the PVDF membranes by probing with a rabbit antibody specific to the sFv portion of the IL-2-sFv fusion protein. A donkey anti-rabbit antibody conjugated to alkaline phosphatase was used as the secondary antibody. The bands were detected using bromo-chloro-indolyl phosphate (BCIP) and Nitro-blue tetrazolium (NBT).

Using such an assay to examine transcytosis, it was possible to recover IL-2-sFv fusion protein from the basal medium, demonstrating that compound underwent transcytosis from the apical to basolateral side of the cells.

A variety of methods and compositions may be used to detect and quantify the IL-2-sFv fusion protein. These include, by way of non-limiting example, a commercially available IL-2 ELISA (DuoSet ELISA Development Kit, R & D Systems, Inc., Minneapolis, Minn.) may be used. A variety of monoclonal antibodies to IL-2 are known and can be used (see for example, Redmond et al., Monoclonal antibodies for purification and assay of IL-2, 17: Lymphokine 5:S29-S34, 1986).

### EXAMPLE 8

### Preparation of Mammalian Expression Constructs Encoding IL-2-sFv Fusion Proteins

An amino terminal fusion of IL-2 with an sFv designed to favor sFv dimer formation was constructed by cloning IL-2, with its signal peptide, into the NheI site of the sFv shown in FIG. 5. A linker consisting of (Gly₂Ser)₂ had previously been ligated to the 5′ end of this sFv.

The following primers were used to amplify IL-2 with its signal sequence from IL-2/5A cloned into pcDNA3.1D V5-His-TOPO.

| | |
|---|---|
| IL2_EcoRV_For (SEQ ID NO:11): | 5′-GATCGATATCATGTACAGGATGCAACTGCTG-3′ |
| IL2_Nhel_Rev (SEQ ID NO:12): | 5′-CGATGCTAGCAGTTAGTGTTGAGATGATGCTTTG-3′ |

Twenty five cycles of PCR were performed at 58° C. The PCR product was cloned into an intermediate vector: pCR-BluntII-TOPO (Invitrogen, Carlsbad, Calif.). The IL-2 PCR product was cut out from this intermediate vector using EcoRV and Nhe1, gel purified and cloned into the Nhe1 site of (Gly₂Ser)₂-sFv in the mammalian expression vector pDIZ. pDIZ was constructed as follows: A 4882 bp Spe1/EcoRV fragment was isolated from pcDNA 3.1 Hygro (Invitrogen, Calif.) and ligated to a Spe1/Xmn1 fragment from gWiz (Gene Therapy Systems Inc.). A plasmid map of pDIZ is shown in FIG. 7.

The DNA was used to transform E. coli, and transformants were selected using ampicillin, as the vector comprises an ampicillin resistance gene. Individual colonies were selected and grown in LB media containing ampicillin. Small scale preparations (mini-preps) of plasmid DNA from 8 colonies were prepared. The nucleotide sequence of the chimeric reading frame that is found in the expression constructs and which encodes the IL-2-APL10 fusion protein was determined in order to confirm the accuracy and fidelity of the PCR reactions.

### EXAMPLE 9

### Biological Activity of IL-2-sFv Conjugates

The IL-2 biological activity of the IL-2-sFv fusion protein was tested by evaluating the ability to sustain proliferation of the IL-2-dependent murine cytotoxic T cell line, CTLL-2 (Melani et al., Targeting of interleukin 2 to human ovarian carcinoma by fusion with a single-chain Fv of antifolate receptor antibody, Cancer Res. 58(18):4146-4154, 1998). The fusion protein supported proliferation of the T cells in this assay in a concentration-dependent manner.

The ability of fusion proteins to bind ligands, such as soluble IL-2-receptor polypeptides (Dracheva et al., Protein Expr. Purif. 6:737-47, 1995; Junghans et al., J. Biol. Chem. 271:10453-60, 1996) or lipoteichoic acid (Plitnick et al., Clin. Diagn. Lab. Immunol. 8(5):972-9, 2001) can be measured either directly when immobilized on a surface or indirectly by their ability to competitively inhibit IL-2 binding to antibody in ELISA assays. Other methods for measuring the amount and biological activity of IL-2 are described by Gately et al. in: Current Protocols in Immunology, John Wiley and Sons, New York, 2000; Indrova et al., Folla Biol. (Praha) 43:45-47, 1997.

### EXAMPLE 10

### Transfection and Expression in Eukaryotic Cells

A large scale preparation of plasmid DNA from one of the sequence-confirmed transformants was prepared and used to transiently transfect CHO cells using LipofectAMINE 2000 (Invitrogen, Calif.) essentially according to the manufacturer's instructions (see Whitt et al., Unit 9.4, pages 9-11 to 9-12, and Unit 16.13, Aruffo, pages 16-53 to 16-55 in: Short Protocols in Molecular Biology, 2nd Ed., Ausubel et al., editors, John Wiley and Sons, New York, 1992). Anti-sFv polyclonal antibody was used to detect fusion proteins containing the sFv polypeptide. Transformants are also screened for production and the secretion of the IL-2-sFv fusion protein by ELISA or Western analysis using antibodies to human IL-2 (Chemicon Inc., CA). Antibodies to human IL-2 are also commercially available from, e.g., Research Diagnostics, Inc. (Flanders, N.J.) and Sigma Chemical Corp. (St. Louis, Mo.). The desired fusion protein is detected by both antibodies. Supernatants from transfected cells were loaded onto a 1 ml Protein A column, which interacts with the VH chain of sFv and permits affinity purification.

### EXAMPLE 11

### Preparation of Mammalian Expression Constructs Encoding sFv-α-Interferon Fusion Proteins

In order to engineer a C-terminal human α-Interferon (α-IFN)-sFv chimeric vector, the α-IFN gene was first isolated from human placental DNA (Sigma, St. Louis, Mo.; cat.# D-4642) by PCR amplification using primers designed from the registered Genbank sequence (accession #J00207). One (1) μg of placental DNA was amplified using Vent DNA polymerase (New England Biolabs, Beverly, Mass.) in a 100 μL reaction using the primers ‘IFNA 091302-1TPF Forward’ (SEQ ID NO: 13) and ‘IFNA 091302-2TPR 2 Reverse’ (SEQ ID NO: 14) as per manufacturer's instructions. The 3-step PCR amplification included 5 cycles with annealing temperature at 50° C. followed by 30 cycles at 55° C.

| | |
|---|---|
| IFNA 091302-1TPF Forward primer (SEQ ID NO:13): | 5′-ATGGCGTTGACCTTTGCGTTACTGGTGGCCCTCCTGGTGCTCA-3′ |
| IFNA 091302-2TPR Reverse primer (SEQ ID NO:14): | 5′-CCAGTTTTCATTCCTTACTTCTTAAACTTTCTTGCAAGT-3′ |

The 100 μl PCR reaction was subjected to gel purification and the 567 bp PCR product purified using a Qiaquick column (Qiagen, Valencia, Calif.). 2 μl of purified product was used for ligating into the pCR 4 Blunt TOPO vector (Invitrogen, Carlsbad, Calif.) using T4 DNA ligase (NEB, Beverly, Mass.) as per manufacture's instructions. Miniprep DNA was prepared (Qiagen miniprep kit cat. #27106) and positive clones sequenced. Clone #6 contained the α-IFN gene and N-terminal signal sequence as follows:

α-IFN gene sequence: (SEQ ID NO:15):

| | | | | | | |
|---|---|---|---|---|---|---|
| ATGGCGTTGA | CCTTTGCGTT | ACTGGTGGCC | CTCCTGGTGC | TCAGCTGCAA | GTCAAGCTGC | 60 |
| TCTGTGGGCT | GTGATCTGCC | TCAAACCCAC | AGCCTGGGTA | GCAGGAGGAC | CTTGATGCTC | 120 |
| CTGGCACAGA | TGAGGAGAAT | CTCTCTTTTC | TCCTGCTTGA | AGGACAGACA | TGACTTTGGA | 180 |
| TTTCCCCAGG | AGGAGTTTGG | CAACCAGTTC | CAAAAGGCTG | AAACCATCCC | TGTCCTCCAT | 240 |
| GAGATGATCC | AGCAGATCTT | CAATCTCTTC | AGCACAAAGG | ACTCATCTGC | TGCTTGGGAT | 300 |
| GAGACCCTCC | TAGACAAATT | CTACACTGAA | CTCTACCAGC | AGCTGAATGA | CCTGGAAGCC | 360 |
| TGTGTGATAC | AGGGGGTGGG | GGTGACAGAG | ACTCCCCTGA | TGAAGGAGGA | CTCCATTCTG | 420 |
| GCTGTGAGGA | AATACTTCCA | AAGAATCACT | CTCTATCTGA | AAGAGAAGAA | ATACAGCCCT | 480 |
| TGTGCCTGGG | AGGTTGTCAG | AGCAGAAATC | ATGAGATCTT | TTTCTTTGTC | AACAAACTTG | 540 |
| CAAGAAAGTT | TAAGAAGTAA | GGAATAA | | | | 567 |

To construct the sFv-α-IFN chimera, 100 ng of pCR 4 Blunt TOPO IFN-a clone #6 template DNA was PCR amplified using Vent DNA polymerase and primers ‘112202-1 TPF AvrII-G4S-IFNA2B Forward’ (SEQ ID NO:16) and ‘112202-2TPR IFN2b NheI-SalI Reverse’ (SEQ ID NO:17):

| | |
|---|---|
| 112202-1TPF AvrII-G4S-IFNA2B Forward | 5′ ACCGTCCTAGGTGGTGGCGGAGGGTCATGTGATCTGCCTCAAACCCACAGCCT-3′ |
| (SEQ ID NO:16): | |
| 112202-2TPR IFN2b NheI-SalI Reverse | 5′-TCCTCGAGGTCGACGCTAGCTTATTATTCCTTACTTCTTAAACTTTCTTGCAAGT-3′ |
| 5 (SEQ ID NO:17): | |

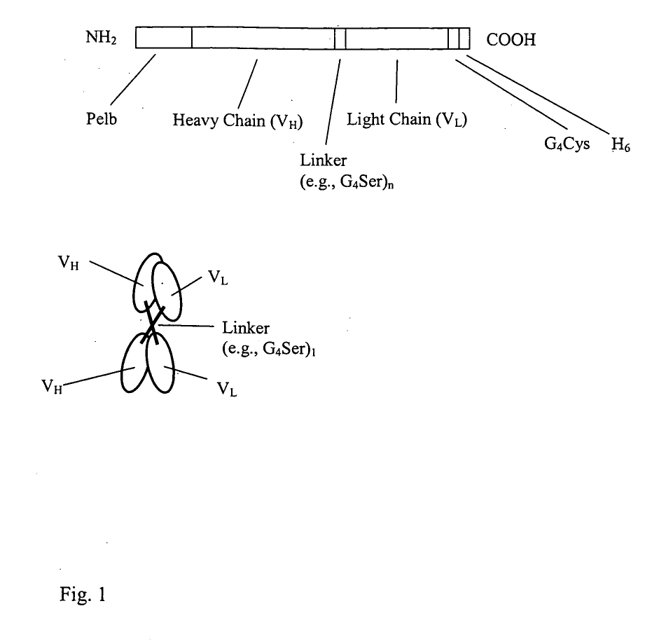

The forward primer used to generate the α-IFN 544 bp PCR product was designed to include sequences encoding a synthetic linker encoding 5 amino acids (Gly-Gly-Gly-Gly-Ser) that are connected in frame to the C-terminus sFv polypeptide. The 3-step PCR amplification reaction included 5 cycles with annealing temperature at 55° C. followed by 30 cycles at 60° C. The 544 bp PCR product was gel purified and cloned into the pCR Blunt II TOPO intermediate vector. Miniprep DNA was made and positives clones verified for the PCR product by DNA sequencing. Following sequence confirmation, the PCR product was excised by digesting the maxiprep DNA with AvrII and SalI restriction enzymes, then ligated into AvrII/SalI digested APL-10 pELK vector DNA using T4 DNA ligase. Miniprep DNA was prepared and positive clones confirmed by DNA sequencing. Positive vector clones are illustrated in FIG. 1 and contain the chimeric DNA sequence (SEQ ID NO: 18) which encodes a chimeric protein containing the following protein domain structural orientation: (NH₂)-pel-B leader-sFv-Gly₄Ser linker-α-IFN —(COOH);

sFv-α-IFN chimera DNA sequence (SEQ ID NO:18):

| | | | | | | |
|---|---|---|---|---|---|---|
| ATGAAATACC | TATTGCCTAC | GGCAGCCGCT | GGATTGTTAT | TACTCGCGGC | CCAGCCGGCC | 60 |
| ATGGCCCAGG | TACAGCTGCA | GCAATCAGGG | GGAGGCGTGG | TCCAGCCTGG | GAGGTCCCTG | 120 |
| AGACTCTCCT | GTGCAGCCTC | TGGATTCACC | TTCAGTAGCT | ATGCTATGCA | CTGGGTCCGC | 180 |
| CAGGCTCCAG | GGAAGGGGCT | GGAGTGGGTC | TCAGCTATTA | GTGGTAGTGG | TGGTAGCACA | 240 |
| TACTACGCAG | ACTCCGTGAA | GGGCCGGTTC | ACCATCTCCA | GAGACAACGC | CAAGAACTCA | 300 |
| CTGTATCTGC | AAATGAACAG | CCTGAGAGCC | GAGGACACGG | CTGTGTATTA | CTGTGCGAGA | 360 |
| GATACCCGAG | GGTACTTCGA | TCTCTGGGGC | CGTGGCACCC | TGGTCACCGT | CTCCTCAGGT | 420 |
| GGCGGAGGGT | CATCTGAGCT | GACTCAGGAC | CCTGCTATGT | CTGTGGCCTT | GGGACAGACA | 480 |
| GTCAGAATCA | CATGTCAAGG | GGACAGTCTC | AGAAAGTATC | ATGCAAGCTG | GTATCAGCAG | 540 |
| AAGCCAGGGC | AGGCCCCTGT | TCTTGTCATC | TATGGTAAGA | ATGAACGTCC | CTCAGGGATC | 600 |
| CCAGAGCGAT | TCTCTGGGTC | CACCTCAGGA | GACACAGCTT | CCTTGACCAT | CAGTGGGCTC | 660 |
| CAGGCGGAAG | ATGAGGCTGA | CTATTACTGT | CACTCCCGAG | ACTCTAATGC | TGATCTTGTG | 720 |
| GTGTTCGGCG | GAGGGACCAA | GGTCACCGTC | CTAGGTGGTG | GCGGAGGGTC | ATGTGATCTG | 780 |
| CCTCAAACCC | ACAGCCTGGG | TAGCAGGAGG | ACCTTGATGC | TCCTGGCACA | GATGAGGAGA | 840 |
| ATCTCTCTTT | TCTCCTGCTT | GAAGGACAGA | CATGACTTTG | GATTTCCCCA | GGAGGAGTTT | 900 |
| GGCAACCAGT | TCCAAAAGGC | TGAAACCATC | CCTGTCCTCC | ATGAGATGAT | CCAGCAGATC | 960 |
| TTCAATCTCT | TCAGCACAAA | GGACTCATCT | GCTGCTTGGG | ATGAGACCCT | CCTAGACAAA | 1020 |
| TTCTACACTG | AACTCTACCA | GCAGCTGAAT | GACCTGGAAG | CCTGTGTGAT | ACAGGGGGTG | 1080 |
| GGGGTGACAG | AGACTCCCCT | GATGAAGGAG | GACTCCATTC | TGGCTGTGAG | GAAATACTTC | 1140 |
| CAAAGAATCA | CTCTCTATCT | GAAAGAGAAG | AAATACAGCC | CTTGTGCCTG | GGAGGTTGTC | 1200 |
| AGAGCAGAAA | TCATGAGATC | TTTTTCTTTG | TCAACAAACT | TGCAAGAAAG | TTTAAGAAGT | 1260 |
| AAGGAATAA | | | | | | 1269 |

While the foregoing examples describe the preparation of sFv-α-IFN conjugates as fusion proteins, the skilled artisan will understand that additional methods (e.g., chemical crosslinking, encapsulation in particles; etc.) may be employed to associate α-IFN with an appropriate targeting element. The sFv-α-IFN construct was expressed in E. coli and purified protein is isolated by FPLC using a Protein-A affinity column as described herein for sFv-Il-2 constructs.

An antiviral bioassay may be used to measure α-IFN activity, based on the ability of α-IFN to protect human foreskin fibroblast FS-71 cells from the cytopathic effects of encephalomyocarditis virus, calibrated against the World Health Organization standard.

### EXAMPLE 12

### Preparation of Mammalian Expression Constructs Encoding sFv-β-Interferon Fusion Proteins

The human α-interferon (β-IFN) gene was isolated from human placental DNA (Sigma, St. Louis, Mo.; cat.# D-4642) by PCR amplification using primers designed from the registered Genbank sequence (accession #M28622). The ‘Human IFN-β15‘ per XhoI-EcoRV-X’ (SEQ ID NO:19) and ‘Human IFN-β1 3’ per X-NheI-stop-BglII-XbaI’ (SEQ ID NO:20) primers were used in the PCR amplification reaction which included 5 cycles with annealing temperature at 55° C. followed by 30 cycles at 60° C.

| | |
|---|---|
| Human IFN-β1 5′pcr primer XhoI-EcoRV-X (SEQ ID NO: 19): | 5′-CCTCGAGATATCGCCACCATGACCAACAAGTGTCTCCTCCA-3′ |
| Human IFN-β1 3′pcr primer X-NheI-stop-BglII-XbaI | 5′-CTCTAGATCTTCAGCTAGCGTTTCGGAGGTAACCTGT-3′ |
| (SEQ ID NO:20): | |

The 100 μl PCR reaction was purified using a QLAquick PCR purification column (cat.# 28104, Qiagen, Valencia, Calif.). 2 μl of purified product was ligated into the pCR II Blunt TOPO vector (Invitrogen, Carlsbad, Calif.). Colonies were picked and miniprep DNA was prepared (Qiagen miniprep kit #27106). Positive clones were confirmed by DNA sequencing. pCR II Blunt TOPO Hum-β-IFN (pCRIIBT HIFNβ) contained the human β-IFN gene and the wild-type N-terminal signal peptide as follows:

β-IFN gene sequence: (SEQ ID NO:21):

| | | | | | | | |
|---|---|---|---|---|---|---|---|
| ATGACCAACA | AGTGTCTCCT | CCAAATTGCT | CTCCTGTTGT | GCTTCTCCAC | TACAGCTCTT | 60 | +TZ,45 |
| TCCATGAGCT | ACAACTTGCT | TGGATTCCTA | CAAAGAAGCA | GCAATTTTCA | GTGTCAGAAG | 120 | |
| CTCCTGTGGC | AATTGAATGG | GAGGCTTGAA | TACTGCCTCA | AGGACAGGAT | GAACTTTGAC | 180 | |
| ATCCCTGAGG | AGATTAAGCA | GCTGCAGCAG | TTCCAGAAGG | AGGACGCCGC | ATTGACCATC | 240 | |
| TATGAGATGC | TCCAGAACAT | CTTTGCTATT | TTCAGACAAG | ATTCATCTAG | CACTGGCTGG | 300 | |
| AATGAGACTA | TTGTTGAGAA | CCTCCTGGCT | AATGTCTATC | ATCAGATAAA | CCATCTGAAG | 360 | |
| ACAGTCCTGG | AAGAAAAACT | GGAGAAAGAA | GATTTCACCA | GGGGAAAACT | CATGAGCAGT | 420 | |
| CTGCACCTGA | AAAGATATTA | TGGGAGGATT | CTGCATTACC | TGAAGGCCAA | GGAGTACAGT | 480 | |
| CACTGTGCCT | GGACCATAGT | CAGAGTGGAA | ATCCTAAGGA | ACTTTTACTT | CATTAACAGA | 540 | |
| CTTACAGGTT | ACCTCCGAAA | CTGA | | | | 564 | |

The β-IFN gene was fused to the N-terminus of APL10 via the NheI site to make pDIZ HIFNβ-APL10. To construct the sFv-β-IFN chimera, 100 ng of pDIZHIFNβ-APL10 was used as the template for PCR amplification using Vent DNA polymerase and the primers ‘1122602-1TPF AvrII-G4S-IFN Beta Forward’ (SEQ ID NO:22) and ‘122602-2TPR IFN Beta NheI-SalI-XhoI Reverse’ (SEQ ID NO:23):

| | |
|---|---|
| 122602-1TPF AvrII-G4S-IFN Beta | |
| Forward | |
| 5′-ACCGTCCTAGGTGGTGGCGGAGGGTCAATGAGCTACAACTTGCTTGGATTCCTA-3′: | (SEQ ID NO:22) |
| 122602-2TPR IFN Beta NheI-SalI-XhoI | |
| Reverse | |
| 5′-TCCTCGAGGTCGACGCTAGCTTATTAGTTTCGGAGGTAACCTGTAAGTCTGTTA-3′: | (SEQ ID NO:23) |

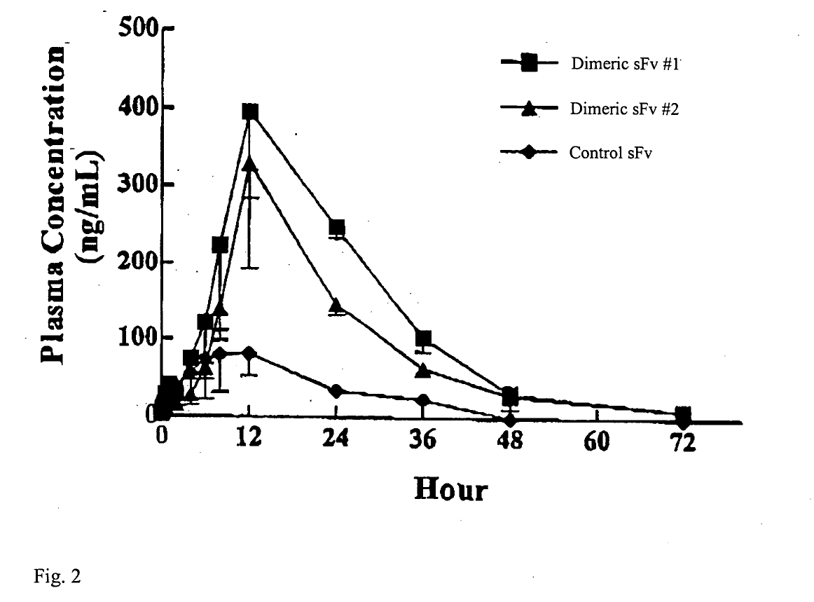

The forward primer used to generate the partial APL-10-β-IFN 551 bp PCR product was designed to include sequences encoding a synthetic linker encoding 5 amino acids (Gly-Gly-Gly-Gly-Ser) that can be inserted in frame to the C-terminus of sFv polypeptide APL-10. The 3-step PCR amplification reaction included 5 cycles with annealing temperature at 55° C. followed by 30 cycles at 60° C. The 551 bp PCR product was QIAquick column purified and cloned into the pCR Blunt II TOPO intermediate vector. Miniprep DNA was made and positives clones verified by DNA sequencing. Following sequence confirmation, the PCR product was inserted into the AvrII/SalI sites of APL-10E vector (pELK vector derivative), or AvrII/XhoI digested APL-2005S vector (pSyn vector derivative) DNAs. Miniprep DNA was prepared and positive clones confirmed by DNA sequencing. Positive vector clones are illustrated in FIG. 2 and contain the chimeric DNA sequence (SEQ ID NO: 24) which encode for a chimeric protein containing the following domains and oriented from the N-terminus: (NH₂)-pel-B leader-sFv-Gly₄Ser linker-β-IFN-(COOH).

sFv-β-IFN chimera DNA sequence (SEQ ID NO: 24):

| | | | | | | |
|---|---|---|---|---|---|---|
| ATGAAATACC | TATTGCCTAC | GGCAGCCGCT | GGATTGTTAT | TACTCGCGGC | CCAGCCGGCC | 60 |
| ATGGCCCAGG | TGCAGCTGCA | GCAATCAGGG | GGAGGCGTGG | TCCAGCCTGG | GAGGTCCCTG | 120 |
| AGACTCTCCT | GTGCAGCCTC | TGGATTCACC | TTCAGTAGCT | ATGCTATGCA | CTGGGTCCGC | 180 |
| CAGGCTCCAG | GGAAGGGGCT | GGAGTGGGTC | TCAGCTATTA | GTGGTAGTGG | TGGTAGCACA | 240 |
| TACTACGCAG | ACTCCGTGAA | GGGCCGGTTC | ACCATCTCCA | GAGACAACGC | CAAGAACTCA | 300 |
| CTGTATCTGC | AAATGAACAG | CCTGAGAGCC | GAGGACACGG | CTGTGTATTA | CTGTGCGAGA | 360 |
| GATACCCGAG | GGTACTTCGA | TCTCTGGGGC | CGTGGCACCC | TGGTCACCGT | CTCCTCAGGT | 420 |
| GGCGGAGGGT | CATCTGAGCT | GACTCAGGAC | CCTGCTATGT | CTGTGGCCTT | GGGACAGACA | 480 |
| GTCAGAATCA | CATGTCAAGG | GGACAGTCTC | AGAAAGTATC | ATGCAAGCTG | GTATCAGCAG | 540 |
| AAGCCAGGGC | AGGCCCCTGT | TCTTGTCATC | TATGGTAAGA | ATGAACGTCC | CTCAGGGATC | 600 |
| CCAGAGCGAT | TCTCTGGGTC | CACCTCAGGA | GACACAGCTT | CCTTGACCAT | CAGTGGGCTC | 660 |
| CAGGCGGAAG | ATGAGGCTGA | CTATTACTGT | CACTCCCGAG | ACTCTAATGC | TGATCTTGTG | 720 |
| GTGTTCGGCG | GAGGGACCAA | GGTCACCGTC | CTAGGTGGTG | GCGGAGGGTC | AATGAGCTAC | 780 |
| AACTTGCTTG | GATTCCTACA | AAGAAGCAGC | AATTTTCAGT | GTCAGAAGCT | CCTGTGGCAA | 840 |
| TTGAATGGGA | GGCTTGAATA | CTGCCTCAAG | GACAGGATGA | ACTTTGACAT | CCCTGAGGAG | 900 |
| ATTAAGCAGC | TGCAGCAGTT | CCAGAAGGAG | GACGCCGCAT | TGACCATCTA | TGAGATGCTC | 960 |
| CAGAACATCT | TTGCTATTTT | CAGACAAGAT | TCATCTAGCA | CTGGCTGGAA | TGAGACTATT | 1020 |
| GTTGAGAACC | TCCTGGCTAA | TGTCTATCAT | CAGATAAACC | ATCTGAAGAC | AGTCCTGGAA | 1080 |
| GAAAAACTGG | AGAAAGAAGA | TTTCACCAGG | GGAAAACTCA | TGAGCAGTCT | GCACCTGAAA | 1140 |
| AGATATTATG | GGAGGATTCT | GCATTACCTG | AAGGCCAAGG | AGTACAGTCA | CTGTGCCTGG | 1200 |
| ACCATAGTCA | GAGTGGAAAT | CCTAAGGAAC | TTTTACTTCA | TTAACAGACT | TACAGGTTAC | 1260 |
| CTCCGAAACT | AA | | | | | |

Expression of sFv-β-IFN in mammalian cells required the use of a suitable signal peptide sequence. The PelB signal peptide is an E. coli signal sequence. For mammalian cells we used the tissue plasminogen activator (TPA) signal peptide (GenBank #NM_{—}033011). The TPA signal peptide was fused to the sFV via PCR primer MG TPA-APL10 5′ primer (SEQ ID NO: 25) and MG APL10 3′ primer (SEQ ID NO: 26).

| | |
|---|---|
| MG TPA-APL10 5′ primer | |
| 5′-GGATATCGCCACCATGGATGCAATGAAGAGAGGGCTCTGCTGTGTGCTGCTGCTGTGTGGAGCAGTCTTCGTTTCGCCCAGCCAGGTACAGCTGCAGCA-3′: | (SEQ ID NO:25) |
| MG APL10 3′ primer | |
| 5′-CGCGGCCGCTCAACCTAGGACGGTGACCTTGGTCCCTCCGCCGAACACCA-3′: | (SEQ ID NO:26) |

The resulting TPA signal peptide (tpa SigP)-APL10 per product was digested with EcoRV and NotI and isolated via agarose gel electrophoresis. The digested tpa-SigP-APL10 was inserted into pgWIZ cut with the same enzymes. Resulting clones of pgWIZtpaSigP-APL10 were screened and one was chosen and sequence verified.

The IFNβ region was amplified by PCR using primers MG sigP(−)HIFNβ5′ (SEQ ID NO:27) and MG HIFNβ3′ (SEQ ID NO:28) and pDIZ HIFNβ-APL10 as a template. The wild-type signal peptide was removed and replaced with a (Gly-Gly-Gly-Ser)×2 linker. The signal peptide minus HIFNβ per product was digested with AvrII and NotI and inserted into pgWIZtpaSigP-APL10 cut with the same enzymes to make pgWIZtpaSigP-APL10-HIFNβ. The resulting products were screened by miniprep and verified by sequencing. To subclone the tpaSigP-APL10-HIFNβ into pDIZ, pgWIZtpaSigP-APL10-HIFNβ was cut with EcoRV and NotI and the tpaSigP-APL10-HIFNβ fragment was gel purified.

| | |
|---|---|
| MG sigP(-)HIFNβ 5′ | |
| 5′-GTCCTAGGTGGCGGCGGAAGCGGCGGAGGCTCCATGAGCTACAACTTGCTTGGATTCCTACAAAGAAGCAGCA-3′: | (SEQ ID NO:27) |
| MG HIFNβ 3′ | |
| 5′-TGCGGCCGCTTAGCTAGCTTATTAGTTTCGGAGGTAACCTGTAAGTCTGTTAATGAAGTAAAAGTTCCT-3′: | (SEQ ID NO:28) |

The tpaSigP-APL10-HIFNβ fragment was inserted into pDIZ cut with EcoRV and NotI to make pDIZtpaSigP-APL10-HIFNβ. The full-length insert was sequenced and verified to be correct.

TPA SigP-APL10-IFNβ (SEQ ID NO:29):

| | | | | | | |
|---|---|---|---|---|---|---|
| ATGGATGCAA | TGAAGAGAGG | GCTCTGCTGT | GTGCTGCTGC | TGTGTGGAGC | AGTCTTCGTT | 50 |
| TCGCCCAGCC | AGGTACAGCT | GCAGCAATCA | GGGGGAGGCG | TGGTCCAGCC | TGGGAGGTCC | 100 |
| CTGAGACTCT | CCTGTGCAGC | CTCTGGATTC | ACCTTCAGTA | GCTATGCTAT | GCACTGGGTC | 150 |
| CGCCAGGCTC | CAGGGAAGGG | GCTGGAGTGG | GTCTCAGCTA | TTAGTGGTAG | TGGTGGTAGC | 200 |
| ACATACTACG | CAGACTCCGT | GAAGGGCCGG | TTCACCATCT | CCAGAGACAA | CGCCAAGAAC | 250 |
| TCACTGTATC | TGCAAATGAA | CAGCCTGAGA | GCCGAGGACA | CGGCTGTGTA | TTACTGTGCG | 300 |
| AGAGATACCC | GAGGGTACTT | CGATCTCTGG | GGCCGTGGCA | CCCTGGTCAC | CGTCTCCTCA | 350 |
| GGTGGCGGAG | GGTCATCTGA | GCTGACTCAG | GACCCTGCTA | TGTCTGTGGC | CTTGGGACAG | 400 |
| ACAGTCAGAA | TCACATGTCA | AGGGGACAGT | CTCAGAAAGT | ATCATGCAAG | CTGGTATCAG | 450 |
| CAGAAGCCAG | GGCAGGCCCC | TGTTCTTGTC | ATCTATGGTA | AGAATGAACG | TCCCTCAGGG | 500 |
| ATCCCAGAGC | GATTCTCTGG | GTCCACCTCA | GGAGACACAG | CTTCCTTGAC | CATCAGTGGG | 550 |
| CTCCAGGCGG | AAGATGAGGC | TGACTATTAC | TGTCACTCCC | GAGACTCTAA | TGCTGATCTT | 600 |
| GTGGTGTTCG | GCGGAGGGAC | CAAGGTCACC | GTCCTAGGTG | GCGGCGGAAG | CGGCGGAGGC | 650 |
| TCCATGAGCT | ACAACTTGCT | TGGATTCCTA | CAAAGAAGCA | GCAATTTTCA | GTGTCAGAAG | 700 |
| CTCCTGTGGC | AATTGAATGG | GAGGCTTGAA | TACTGCCTCA | AGGACAGGAT | GAACTTTGAC | 750 |
| ATCCCTGAGG | AGATTAAGCA | GCTGCAGCAG | TTCCAGAAGG | AGGACGCCGC | ATTGACCATC | 800 |
| TATGAGATGC | TCCAGAACAT | CTTTGCTATT | TTCAGACAAG | ATTCATCTAG | CACTGGCTGG | 850 |
| AATGAGACTA | TTGTTGAGAA | CCTCCTGGCT | AATGTCTATC | ATCAGATAAA | CCATCTGAAG | 900 |
| ACAGTCCTGG | AAGAAAAACT | GGAGAAAGAA | GATTTCACCA | GGGGAAAACT | CATGAGCAGT | 950 |
| CTGCACCTGA | AAAGATATTA | TGGGAGGATT | CTGCATTACC | TGAAGGCCAA | GGAGTACAGT | 1000 |
| CACTGTGCCT | GGACCATAGT | CAGAGTGGAA | ATCCTAAGGA | ACTTTTACTT | CATTAACAGA | 1050 |
| CTTACAGGTT | ACCTCCGAAA | CTAA | | | | 1074 |

One correct clone was chosen and plasmid DNA (pDNA) obtained by Qiagen Maxiprep. The DNA (pDIZ-tpa SigP-APL10-IFNβ) was transfected into CHO dhfr(−) cells with Lipofectamine 2000 (Invitrogen) and AZ-IFBC protein expression and secretion was examined after 3 days by western blot. We used the anti-human IFN-β, monoclonal antibody (R&D systems, cat.#MAB814). The protein was applied to a 1 ml protein A sepharose column to examine purification potential. The purified AZ-IFBC was assayed for binding to Rat D6 as described above for functionality of the APL10 domain. The IFNβ domain was examined by inhibition of virus-induced (vesicular somatitus virus, VSV) cytopathic effect (cpe) as described below.

While the foregoing example describes the preparation of sFv-β-IFN conjugates as fusion proteins, the skilled artisan will understand that additional methods (e.g., chemical crosslinking, encapsulation in particles, etc.) may be employed to associate β-IFN with an appropriate targeting element. The sFv-β-IFN was expressed in E. coli and mammalian CHO-dhfr(−) cells. The expressed sFv-β-IFN was purified by FPLC using a Protein-A-sepharose affinity column as described herein for sFv-IL-2.

β-IFN activity may be determined using the cytopathic effect inhibition assay as previously described (Rubinstein, S., Familletti, P. C., and Pestka, S. (1981) “Convenient Assay for Interferons,” J. Virol. 37, 755-758; Familletti, P. C., Rubinstein, S., and Pestka, S. (1981)” A Convenient and Rapid Cytopathic Effect Inhibition Assay for Interferon,” in Methods in Enzymology, Vol. 78 (S. Pestka, ed.), Academic Press, New York, 387-394). In the antiviral assays for β-IFN, about 1 unit/ml of β-IFN is the quantity necessary to protect 50% of the cell culture monolayer. The units are determined with respect to the international reference standard for β-IFN provided by the National Institutes of Health (Pestka, S. (1986)” Interferon Standards and General Abbreviations, in Methods in Enzymology (S. Pestka, ed.), Academic Press, New York 119, 14-23).

### EXAMPLE 13

### Preparation of Expression Constructs Encoding sFv-I-TAC Fusion Proteins

PBMC are stimulated with interferon-alpha for 3 hours and then total RNA, cDNA is made as outlined in the earlier examples. PCR amplification is used to join amplified I-TAC to APL10 coding sequence with a Gly4Ser linker.

Sequences encoding I-TAC with its native leader sequence and APL10 are amplified via PCR with the following primers:

| | | |
|---|---|---|
| ITAC_FOR: | GACT GAT ATC GCC ACC ATG AGT GTG AAG GGC ATG GCT | (SEQ ID NO:30) |
| ITAC_REV: | ATC AAA AAA GTT GAA AGA AAG AAT TTT GGG GGT GGA GGC AGC | (SEQ ID NO:31) |
| REV COMP: | GCT GCC TCC ACC CCC AAA ATT CTT TCT TTC AAC TTT TTT GAT | (SEQ ID NO:32) |
| APL_FOR: | GGG GGT GGA GGC AGC CAG GTA CAG CTG CAG CAA TCA | (SEQ ID NO:33) |
| APL_REV: | C AAG GTC ACC GTC CTA GGT TAA GCG GCC GC | (SEQ ID NO:34) |
| REV COMP: | GCG GCC GCT TAA CCT AGG ACG GTG ACC TTG | (SEQ ID NO:35) |

PCR is performed at about 60° C. for 25 cycles.

The sequence of I-TAC (GENBANK accession number AF30514; coding sequence underlined) is as follows (SEQ ID NO: 36):

| | |
|---|---|
| 1 | ctccttccaa gaagagcagc aaagctgaag tagcagcaac agcaccagca gcaacagcaa |
| 61 | aaaacaaac atgagtgtgaa gggcatggct atagccttgg ctgtgatatt gtgtgctaca |
| 121 | gttgttcaag gcttccccat gttcaaaaga ggacgctgtc tttgcatagg ccctggggta |
| 181 | aaagcagtga aagtggcaga tattgagaaa gcctccataa tgtacccaag taacaactgt |
| 241 | gacaaaatag aagtgattat taccctgaaa gaaaataaag gacaacgatg cctaaatccc |
| 301 | aaatcgaagc aagcaaggct tataatcaaa aaagttgaaa gaaagaattt ttaaaaatat |
| 361 | caaaacatat gaagtcctgg aaaagggcat ctgaaaaacc tagaacaagt ttaactgtga |
| 421 | ctactgaaat gacaagaatt ctacagtagg aaactgagac ttttctatgg ttttgtgact |
| 481 | ttcaactttt gtacagttat gtgaaggatg aaaggtgggt gaaaggacca aaaacagaaa |
| 541 | tacagtcttc ctgaatgaat gacaatcaga attccactgc ccaaaggagt ccagcaatta |
| 601 | aatggatttc taggaaaagc taccttaaga aaggctggtt accatcggag tttacaaagt |
| 661 | gctttcacgt tcttacttgt tgtattatac attcatgcat ttctaggcta gagaaccttc |
| 721 | tagatttgat gcttacaact aitctgttgt gactatgaga acatttctgt ctctagaagt |
| 781 | tatctgtctg tattgatctt tatgctatat tactatctgt ggttacagtg gagacattga |
| 841 | cattattact ggagtcaagc ccttataagt caaaagcatc tatgtgtcgt aaagcattcc |
| 901 | tcaaacattt tttcatgcaa atacacaytt ctttccccaa atatcatgta gcacatcaat |
| 961 | atgtagggaa acattcttat gcatcatttg gtttgtttta taaccaattc attaaatgta |
| 1021 | attcataaaa tgtactatga aaaaaattat acgctatggg atactggcaa cagtgcacat |
| 1081 | atttcataac caaattagca gcaccggtct taatttgatg tttttcaact tttattcatt |
| 1141 | gagatgtttt gaagcaatta ggatatgtgt gtttactgta ctttttgttt tgatccgttt |
| 1201 | gtataaatga tagcaatatc ttggacacat ttgaaataca aaatgttttt gtctaccaaa |
| 1261 | gaaaaatgtt gaaaaataag caaatgtata cctagcaatc acttttactt tttgtaattc |
| 1321 | tgtctcttag aaaaatacat aatctaatca aaaaaaaaaa aaaaaaaaaa a |

PCR products are then cloned into appropriate expression vectors as described in the foregoing examples. The functional activity of recombinant I-TAC fusion proteins is then evaluated using an in vitro chemotaxis assay using a modified Boyden chamber as is known in the art; with target cells being PHA-stimulated T lymphocytes cultured with IL-2 for 8-14 days.

### EXAMPLE 14

### Animal Instillation Studies

FIG. 1 shows the schematic structure of the sFv directed to a pIgR epitope used for the following in vivo transport studies. Indicated are the Pelb leader (a leader sequence that directs secretion from E. coli); linker (amino acid sequence (gly-gly-gly-gly-ser)ₙ); H₆, (6×His tag); cysteine tag (amino acid sequence gly-gly-gly-gly-cys); and the heavy and light chains of the sFv. The selected sFv comprises an altered FR2 region, an internal unpaired cysteine, a C-terminal His tag, and a single linker repeat. This construct directs near homogenous dimeric sFv formation.

The “diabody” sFv directed to a pIgR epitope and prepared according to the previous Examples was administered to rats and/or Cynomolgus (Macca fascicularis) monkeys. For administration to rats, the trachea was exposed with a small incision and a fine needle was inserted between rings in the trachea, but in some experiments, a tube was inserted through the mouth into the trachea of rats. For monkey administration, Cynomolgus monkeys were anesthetized with ketamine (10 mg/kg, IM). A single dose of compound was instilled into the upper bronchus of the right lung using a pediatric fiberoptic bronchoscope. The dose was infused at a rate of approximately 1 ml per minute. Dose volumes were maintained at 0.5 ml/kg. The formulation also contained 1 mg/ml of bovine serum albumin (BSA) as a carrier protein.

Blood samples were collected at various times and plasma prepared from the blood. Plasma concentrations of the compound were detected using an assay formatted in two different ways. In the first assay, GST-domain 6 (which contains the pIgR stalk) was used to capture the compound specifically (an active binding site is required on the compound) and detection was achieved using a polyclonal antibody that recognizes the compound. In the second format, both capture and detection were achieved using polyclonal antibody against the compound (the sandwich assay). In this format, the antibody combining site does not necessarily need to be functional, but the molecule must be otherwise intact.

All of the recombinant proteins used were formulated in either HBSS buffer or HSN buffer. HBSS buffer contains 1.26 mM CaCl₂, 5.36 mM KCl, 156.9 mM NaCl, 25 mM D-glucose, 22.9 mM HEPES, 1.64 mM MgSO₄, 0.44 mM KH₂PO₄, 0.62 mM Na₂HPO₄, 4.35 mM NaHCO₃, adjusted to pH 7.0. HSN buffer contains 150 mM NaCl, 50 mM HEPES, and 146 mM sucrose, at a pH of 7.0. The calculated osmolarity is 545 mOsm. Physiological osmolarity is approximately 300 mOsm.

The half-life of the compound was measured by injecting intravenously 0.8 mg of the compound and determining the plasma concentration as a function of time. A nearly 4 log decrease in the concentration of delivered agent in plasma and bile was observed over 24 hours. The bile duct of the monkeys was cannulated so samples could be collected and analyzed for the presence of compound, and it was determined that compound was not present in bile in significant amounts.

### EXAMPLE 15

### Monkey Studies with pIgR Stalk sFv

A second monkey experiment was designed to verify the results obtained in the previous Example (designated AZ 1), by comparison to a second compound (designated AZ2) and a negative control. The negative control was an antibody fragment directed against c-erbB-2, which does not recognize pIgR. c-erbB-2 is an oncogene product that may be expressed in lung at low levels. Nine monkeys were used and they were divided into three groups with three monkeys in each group. The first group received AZ1 (1 mg/kg), the second received 1 mg/kg of AZ2, and the third received the negative control (1 mg/kg). All three ligands have the same molecular weight (56 kD). Each compound was administered using a pediatric bronchscope aimed at the upper bronchi.

FIG. 2 shows that compound AZ1 was transported into the blood with a Tmax of 12 hours. Furthermore, the average bioavailability calculated was 35.6+−9.6%. In the previous Example study, two monkeys that received the compound in the upper trachea showed a lower Cmax compared to the two monkeys dosed in the bronchia. This disparity lowered the overall average bioavailability, which may be associated with the expected faster clearance by the mucociliary clearance mechanism.

The results shown in FIG. 2 also demonstrated that the AZ2 analogue was transported into the blood following IT administration. The average Cmax obtained was 329+−45 ng/ml and Tmax was reached at 12 hours. These pharmacokinetic parameters Were not significantly different from the results obtained with AZ1 (average Cmax=397+−202 ng/ml and Tmax=12 hours). In contrast, the negative control, which does not bind pIgR, was transported to a lesser degree following intra-tracheal administration. The average Cmax for the negative control was 80+−48 ng/ml and the Tmax was reached by 8 hours. These results show that the negative control was transported by a different mechanism than that of the AZ1 and AZ2 compounds.

### EXAMPLE 16

### Monkey Aerosol Administration Studies

The “diabody” sFv directed to a pIgR epitope and prepared according to Example 5 was also administered Cynomolgus monkeys as an aerosol formulation. In this Example, an Aeroneb Pro nebulizer (aerogen, Inc., Sunnyvale, Calif.) was used to aerosolize a liquid formulation of sFv. Aerosol generation was performed during the inspiratory phase of the recipient animal's respiratory cycle, and was delivered through an endotracheal tube. Anesthesia was induced in the subject animals with an IV bolus of propofol (8-10 mg/kg) and maintained by IV infusion of 0.4 mg/kg/min of the same anesthetic. Subject animals were placed in an iron lung (a “Spangler Box”) to control the respiratory cycle of the animal.

Animals were divided into three exposure groups:

| Group | Total Inhaled Dose | PSD | % Vital Capacity | Breath Hold |
|---|---|---|---|---|
| 1 | 1.5 mg/kg | 2-3 μm | 75% | yes |
| 2 | 1.5 mg/kg | 2-3 μm | 40% | no |
| 3 | 5 mg/kg | | 75% | yes |

The respiratory cycle was fixed at 6-8 breaths per minute, and each animal was exposed to a sufficient number of inspirations to deliver the target dose. 1.5 mg/kg dosages were selected to achieve a 1 mg/kg inhaled dose of sFv. PSD refers to the particle size distribution of the aerosolized material; % vital capacity refers to the size of the tidal volume as a percentage of vital capacity. Group 1 and 3 animals were exposed to a 4-second breath hold on each inspiration during delivery.

1.5 mL blood samples were collected from a peripheral vein from study animals. Samples were collected prior to exposure, and about 1, 2, 4, 6, 8, 12, 18, 24, 36, 48, and 72 hours following exposure.

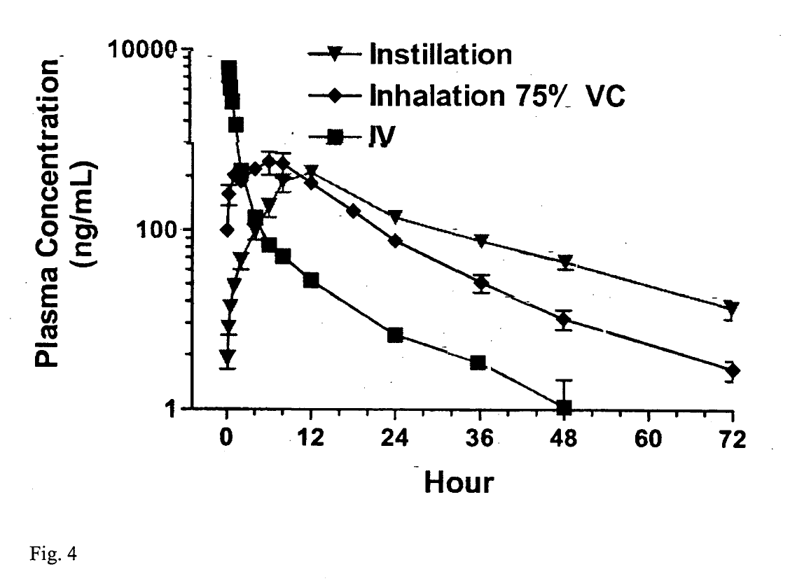

Plasma concentrations achieved at 75% and 40% tidal volumes are shown in FIG. 3. The resulting bioavailability achieved was 45.4% and 27.1% for 75% and 40% tidal volumes, respectively. A comparison of aerosol, instillation, and IV delivery (FIG. 4) shows that both methods of pulmonary delivery of sFv directed to pIgR can provide effective apical to basolateral delivery of agent.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method of treating or preventing a lung disease in a subject comprising, administering to the subject via a pulmonary, oropharyngeal, or nasopharyngeal route a compound comprising a therapeutic agent and a targeting element directed to a ligand, wherein the targeting element confers apical to basolateral transcytosis to the therapeutic agent in an in vitro transcytotic assay.

2. The method of claim 1, wherein the ligand is selected from the group consisting of pIgR, pIgR stalk, transferrin receptor, apo-transferrin, holo-transferrin, vitamin B12 receptor, FcRn, an integrin, Flt-1, Flk-1, Flt-4, a GPI-linked protein, a scavenger receptor, folate receptor, and low density lipoprotein receptor.

3. The method of claim 2, wherein the ligand is the pIgR stalk.

4. The method of claim 2, wherein the targeting element binds a non-secretory component region of pIgR.

5. The method of claim 1, wherein the therapeutic agent is a polypeptide or a nucleic acid.

6. The method of claim 5, wherein the therapeutic agent is an immune system modulator.

7. The method of claim 5, wherein the therapeutic agent is selected from the group consisting of an anti-tumor agent, an anti-infective agent, an anti-angiogenesis agent, and an apoptosis inducer.

8. The method of claim 5, wherein the therapeutic agent is selected from the group consisting of an enzyme, an interleukin, an interferon, a cytokine, a chemokine, TNF, taxol, an antibody, and combinations of any two or more thereof.

9. The method of claim 8, wherein the therapeutic agent is selected from the group consisting of IL-2, IL-3, IL-4, IL-5, IL-6, IL-7, IL-9, IL-12, IL-13, IL-15, interferon a, interferon p, interferon-γ, IP-10, I-TAC, MIG, functional derivatives of any thereof, and combinations of any two or more thereof.

10. The method of claim 9, wherein the therapeutic agent is selected from the group consisting of IL-2, interferon a, interferon p, and functional derivatives of any thereof.

11. The method of claim 1, wherein the compound is administered through inhalation.

12. A method according to claim 1, wherein the composition is administered in a form selected from the group consisting of liquid particles and solid particles.

13. A method according to claim 12, wherein the composition is administered as liquid particles having an average size of between about 1 μm and about 20 μm.

14. A method according to claim 13, wherein the composition is administered as liquid particles having an average size of between about 1 μm and about 10 μm.

15. The method of claim 1, wherein the compound, or a therapeutic portion thereof, is delivered into the lung with a pharmacokinetic profile that results in the delivery of an effective dose of the compound or a therapeutic portion thereof.

16. The method of claim 1, wherein at least 10% of the compound, or a therapeutic portion or metabolite thereof, administered to the subject undergoes apical to basolateral transcytosis from the pulmonary lumen.

17. The method of claim 15, wherein at least 20% of the compound, or a therapeutic portion or metabolite thereof, administered to the subject undergoes apical to basolateral transcytosis from the pulmonary lumen.

18. The method of claim 1, wherein the targeting element is selected from the group consisting of a polypeptide, a recombinant polypeptide, an antibody, an antibody fragment, a single-chain variable region fragment, a small molecule, an oligonucleotide, an oligosaccharide, a polysaccharide, a carbohydrate, a cyclic polypeptide, a peptidomimetic, and an aptamer.

19. The method of claim 1, wherein the lung disease is a primary tumor of the lung.

20. The method of claim 1, wherein the lung disease is a pulmonary metastasis from a primary tumor.

21. The method of claim 20, wherein the primary tumor is selected from the group consisting of a sarcoma, an adenocarcinoma, a choriocarcinoma, and a melanoma.

22. The method of claim 21, wherein the primary tumor is selected from the group consisting of a colon adenocarcinoma, a breast adenocarcinoma, an Ewing's sarcoma, an osteosarcoma and a renal cell carcinoma.

23. The method of claim 20, wherein the primary tumor is a renal cell carcinoma.

24. The method of claim 20, wherein the clinical presentation of the pulmonary metastasis is selected from the group consisting of a solitary metastasis, a cannonball, a lymphangitis carcinoimatosa, and a pleural effusion.

25. The method of claim 1, wherein the lung disease is a respiratory tract infection.

26. The method of claim 1, wherein the lung disease is an infection of the lung.

27. The method of claim 1, wherein the lung disease is a bacterial infection.

28. The method of claim 27, wherein the bacterial infection causes tuberculosis.

29. The method of claim 1, wherein the lung disease is a viral infection.

30. The method of claim 29, wherein the viral infection causes severe acute respiratory syndrome (SARS).

31. The method of claim 1, wherein the lung disease is a fungal infection.

32. The method of claim 1, wherein the lung disease causes pneumonia.

33. The method of claim 1, wherein the lung disease is a disorder of the interstitium.

34. The method of claim 1, wherein the lung disease is a disorder of gas exchange or blood circulation.

35. The method of claim 1, wherein the lung disease is a disease of the airways.

36. The method of claim 1, wherein the lung disease is a disorder of the pleura.

37. The method of claim 1, wherein the lung disease is COPD.

38. The method of claim 1, wherein the lung disease is asthma.

39. The method of claim 1, further comprising administering to the subject a second therapeutic agent.

40. The method of claim 1, further comprising administering to the subject a vaccine directed against an infective agent.

41. The method of claim 1, further comprising administering to the subject a vaccine directed against a cancerous agent or a vaccine directed against a cancer-associated polypeptide.

42. The method of claim 2, wherein the targeting element binds to an epitope on pIgR or pIgR stalk that comprises an amino acid sequence selected from the group consisting of LRKED (SEQ ID NO: 37), QLFVNEE (SEQ ID NO: 38), LNQLT (SEQ ID NO: 39), YWCKW (SEQ ID NO: 40), GWYWC (SEQ ID NO: 41), STLVPL (SEQ ID NO: 42), SYRTD (SEQ ID NO: 43), QDPRLF (SEQ ID NO: 44) and KRSSK (SEQ ID NO: 45).

43. The method of claim 2, wherein the targeting element binds to pIgR or pIgR stalk in a region selected from the group consisting of:
R1 From KRSSK (SEQ ID NO: 45) to the carboxy terminus of pIgR,
R2a From SYRTD (SEQ ID NO: 43) to the carboxy terminus of pIgR,
R2b From SYRTD (SEQ ID NO: 43) to KRSSK (SEQ ID NO: 45),
R3a From STLVPL (SEQ ID NO: 42) to the carboxy terminus of pIgR,
R3b From STLVPL (SEQ ID NO: 42) to KRSSK (SEQ ID NO: 45),
R3c From STLVPL (SEQ ID NO: 42) to SYRTD (SEQ ID NO: 43),
R4a From GWYWC (SEQ ID NO: 41) to the carboxy terminus of pIgR,
R4b From GWYWC (SEQ ID NO: 41) to KRSSK (SEQ ID NO: 45),
R4c From GWYWC (SEQ ID NO: 41) to SYRTD (SEQ ID NO: 43),
R4d From GWYWC (SEQ ID NO: 41) to STLVPL (SEQ ID NO: 42),
R5a From YWCKW (SEQ ID NO: 40) to the carboxy terminus of pIgR,
R5b From YWCKW (SEQ ID NO: 40) to KRSSK (SEQ ID NO: 45),
R5c From YWCKW (SEQ ID NO: 40) to SYRTD (SEQ ID NO: 43),
R5d From YWCKW (SEQ ID NO: 40) to STLVPL (SEQ ID NO: 42),
R5e From YWCKW (SEQ ID NO: 40) to GWYWC (SEQ ID NO: 41),
R6a From LNQLT (SEQ ID NO: 39) to the carboxy terminus of pIgR,
R6b From LNQLT (SEQ ID NO: 39) to KRSSK (SEQ ID NO: 45),
R6c From LNQLT (SEQ ID NO: 39) to SYRTD (SEQ ID NO: 43),
R6d From LNQLT (SEQ ID NO: 39) to STLVPL (SEQ ID NO: 42),
R6e From LNQLT (SEQ ID NO: 39) to GWYWC (SEQ ID NO: 41),
R6f From LNQLT (SEQ ID NO: 39) to YWCKW (SEQ ID NO: 40),
R7a From QLFVNEE (SEQ ID NO: 38) to the carboxy terminus of pIgR,
R7b From QLFVNEE (SEQ ID NO: 38) to KRSSK (SEQ ID NO: 45),
R7c From QLFVNEE (SEQ ID NO: 38) to SYRTD (SEQ ID NO: 43),
R7d From QLFVNEE (SEQ ID NO: 38) to STLVPL (SEQ ID NO: 42),
R7e From QLFVNEE (SEQ ID NO: 38) to GWYWC (SEQ ID NO: 41),
R7f From QLFVNEE (SEQ ID NO: 38) to YWCKW (SEQ ID NO: 40),
R7g From QLFVNEE (SEQ ID NO: 38) to LNQLT (SEQ ID NO: 39),
R8a From LRKED (SEQ ID NO: 37) to the carboxy terminus of pIgR,
R8b From LRKED (SEQ ID NO: 37) to KRSSK (SEQ ID NO: 45),
R8c From LRKED (SEQ ID NO: 37) to SYRTD (SEQ ID NO: 43),
R8d From LRKED (SEQ ID NO: 37) to STLVPL (SEQ ID NO: 42),
R8e From LRKED (SEQ ID NO: 37) to GWYWC (SEQ ID NO: 41),
R8f From LRKED (SEQ ID NO: 37) to YWCKW (SEQ ID NO: 40),
R8g From LRKED (SEQ ID NO: 37) to LNQLT (SEQ ID NO: 39), and
R8h From LRKED (SEQ ID NO: 37) to QLFVNEE (SEQ ID NO: 38).

44. The method of claim 1, wherein the compound further comprises a PTD or MTS.

45. The method of claim 1, wherein the compound further comprises a second targeting element.

46. The method of claim 45, wherein the second targeting element is substantially identical to the first targeting element.

47. The method of claim 1, wherein the targeting element comprises two to four binding sites for the ligand.

48. The method of claim 47, wherein the targeting element is selected from the group consisting of an antibody, an Fab fragment, and a single chain variable region fragment (sFv) diabody.

49. The method of claim 1, wherein the targeting element comprises two to four single chain variable region fragments (sFv), each sFv comprising a heavy chain variable domain covalently linked, directly or through a polypeptide linker, to a light chain variable domain, wherein one or more of the sFvs is covalently or noncovalently associated with the therapeutic agent.

50. The method of claim 49, wherein at least one sFv binds to pIgR.

51. The method of claim 50, wherein at least one sFv binds to a non-secretory component region of pIgR.

52. The method of claim 50, wherein at least one sFv binds to pIgR stalk.


SEQUENCE LISTING (54 sequences)

SEQ ID NO: 1 - DNA, 30 bp, Artificial Sequence
  Description of Artificial Sequence Primer
caccatgtacaggatgcaactgctgtcttg

SEQ ID NO: 2 - DNA, 51 bp, Artificial Sequence
  Description of Artificial Sequence Primer
gatttgccgctaccggaagtcgacccagttagtgttgagatgatgctttga

SEQ ID NO: 3 - DNA, 794 bp, Homo sapiens
tcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaa

SEQ ID NO: 4 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaacttga

SEQ ID NO: 5 - protein, 13 aa, Artificial Sequence
  Description of Artificial Sequence Spacer peptide
GSTSGSGKSSEGK

SEQ ID NO: 6 - DNA, 53 bp, Artificial Sequence
  Description of Artificial Sequence Primer
gtagcggcaaatcctctgaaggcaaacaggtgcagctggtgcaatcaggggga

SEQ ID NO: 7 - DNA, 24 bp, Artificial Sequence
  Description of Artificial Sequence Primer
acctaggacggtgaccttggtccc

SEQ ID NO: 8 - DNA, 59 bp, Artificial Sequence
  Description of Artificial Sequence Primer
gatccctaggtggcggcggaagcggcggaggctccgcacctacttcaagttctacaaag

SEQ ID NO: 9 - DNA, 38 bp, Artificial Sequence
  Description of Artificial Sequence Primer
ctcgagttattaagttagtgttgagatgatgctttgac

SEQ ID NO: 10 - protein, 14 aa, Artificial Sequence
  Description of Artificial Sequence Synthetic peptide
EGKSSGSGSESKEF

SEQ ID NO: 11 - DNA, 31 bp, Artificial Sequence
  Description of Artificial Sequence Primer
gatcgatatcatgtacaggatgcaactgctg

SEQ ID NO: 12 - DNA, 34 bp, Artificial Sequence
  Description of Artificial Sequence Primer
cgatgctagcagttagtgttgagatgatgctttg

SEQ ID NO: 13 - DNA, 43 bp, Artificial Sequence
  Description of Artificial Sequence Primer
atggcgttgacctttgcgttactggtggccctcctggtgctca

SEQ ID NO: 14 - DNA, 39 bp, Artificial Sequence
  Description of Artificial Sequence Primer
ccagttttcattccttacttcttaaactttcttgcaagt

SEQ ID NO: 15 - DNA, 567 bp, Homo sapiens
atggcgttgacctttgcgttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaataa

SEQ ID NO: 16 - DNA, 53 bp, Artificial Sequence
  Description of Artificial Sequence Primer
accgtcctaggtggtggcggagggtcatgtgatctgcctcaaacccacagcct

SEQ ID NO: 17 - DNA, 55 bp, Artificial Sequence
  Description of Artificial Sequence Primer
tcctcgaggtcgacgctagcttattattccttacttcttaaactttcttgcaagt

SEQ ID NO: 18 - DNA, 1269 bp, Artificial Sequence
  Description of Artificial Sequence sFv-alpha-IFN chimera nucleotide sequence
atgaaatacctattgcctacggcagccgctggattgttattactcgcggcccagccggccatggcccaggtacagctgcagcaatcagggggaggcgtggtccagcctgggaggtccctgagactctcctgtgcagcctctggattcaccttcagtagctatgctatgcactgggtccgccaggctccagggaaggggctggagtgggtctcagctattagtggtagtggtggtagcacatactacgcagactccgtgaagggccggttcaccatctccagagacaacgccaagaactcactgtatctgcaaatgaacagcctgagagccgaggacacggctgtgtattactgtgcgagagatacccgagggtacttcgatctctggggccgtggcaccctggtcaccgtctcctcaggtggcggagggtcatctgagctgactcaggaccctgctatgtctgtggccttgggacagacagtcagaatcacatgtcaaggggacagtctcagaaagtatcatgcaagctggtatcagcagaagccagggcaggcccctgttcttgtcatctatggtaagaatgaacgtccctcagggatcccagagcgattctctgggtccacctcaggagacacagcttccttgaccatcagtgggctccaggcggaagatgaggctgactattactgtcactcccgagactctaatgctgatcttgtggtgttcggcggagggaccaaggtcaccgtcctaggtggtggcggagggtcatgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaataa

SEQ ID NO: 19 - DNA, 41 bp, Artificial Sequence
  Description of Artificial Sequence Primer
cctcgagatatcgccaccatgaccaacaagtgtctcctcca

SEQ ID NO: 20 - DNA, 37 bp, Artificial Sequence
  Description of Artificial Sequence Primer
ctctagatcttcagctagcgtttcggaggtaacctgt

SEQ ID NO: 21 - DNA, 564 bp, Homo sapiens
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 22 - DNA, 54 bp, Artificial Sequence
  Description of Artificial Sequence Primer
accgtcctaggtggtggcggagggtcaatgagctacaacttgcttggattccta

SEQ ID NO: 23 - DNA, 54 bp, Artificial Sequence
  Description of Artificial Sequence Primer
tcctcgaggtcgacgctagcttattagtttcggaggtaacctgtaagtctgtta

SEQ ID NO: 24 - DNA, 1272 bp, Artificial Sequence
  Description of Artificial Sequence sFv-beta-IFN chimera nucleotide sequence
atgaaatacctattgcctacggcagccgctggattgttattactcgcggcccagccggccatggcccaggtgcagctgcagcaatcagggggaggcgtggtccagcctgggaggtccctgagactctcctgtgcagcctctggattcaccttcagtagctatgctatgcactgggtccgccaggctccagggaaggggctggagtgggtctcagctattagtggtagtggtggtagcacatactacgcagactccgtgaagggccggttcaccatctccagagacaacgccaagaactcactgtatctgcaaatgaacagcctgagagccgaggacacggctgtgtattactgtgcgagagatacccgagggtacttcgatctctggggccgtggcaccctggtcaccgtctcctcaggtggcggagggtcatctgagctgactcaggaccctgctatgtctgtggccttgggacagacagtcagaatcacatgtcaaggggacagtctcagaaagtatcatgcaagctggtatcagcagaagccagggcaggcccctgttcttgtcatctatggtaagaatgaacgtccctcagggatcccagagcgattctctgggtccacctcaggagacacagcttccttgaccatcagtgggctccaggcggaagatgaggctgactattactgtcactcccgagactctaatgctgatcttgtggtgttcggcggagggaccaaggtcaccgtcctaggtggtggcggagggtcaatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaa

SEQ ID NO: 25 - DNA, 99 bp, Artificial Sequence
  Description of Artificial Sequence Primer
ggatatcgccaccatggatgcaatgaagagagggctctgctgtgtgctgctgctgtgtggagcagtcttcgtttcgcccagccaggtacagctgcagca

SEQ ID NO: 26 - DNA, 50 bp, Artificial Sequence
  Description of Artificial Sequence Primer
cgcggccgctcaacctaggacggtgaccttggtccctccgccgaacacca

SEQ ID NO: 27 - DNA, 73 bp, Artificial Sequence
  Description of Artificial Sequence Primer
gtcctaggtggcggcggaagcggcggaggctccatgagctacaacttgcttggattcctacaaagaagcagca

SEQ ID NO: 28 - DNA, 69 bp, Artificial Sequence
  Description of Artificial Sequence Primer
tgcggccgcttagctagcttattagtttcggaggtaacctgtaagtctgttaatgaagtaaaagttcct

SEQ ID NO: 29 - DNA, 1284 bp, Artificial Sequence
  Description of Artificial Sequence TPA SigP-APL10-IFN-beta
atggatgcaatgaagagagggctctgctgtgtgctgctgctgtgtggagcagtcttcgtttcgcccagccaggtacagctgcagcaatcagggggaggcgtggtccagcctgggaggtccctgagactctcctgtgcagcctctggattcaccttcagtagctatgctatgcactgggtccgccaggctccagggaaggggctggagtgggtctcagctattagtggtagtggtggtagcacatactacgcagactccgtgaagggccggttcaccatctccagagacaacgccaagaactcactgtatctgcaaatgaacagcctgagagccgaggacacggctgtgtattactgtgcgagagatacccgagggtacttcgatctctggggccgtggcaccctggtcaccgtctcctcaggtggcggagggtcatctgagctgactcaggaccctgctatgtctgtggccttgggacagacagtcagaatcacatgtcaaggggacagtctcagaaagtatcatgcaagctggtatcagcagaagccagggcaggcccctgttcttgtcatctatggtaagaatgaacgtccctcagggatcccagagcgattctctgggtccacctcaggagacacagcttccttgaccatcagtgggctccaggcggaagatgaggctgactattactgtcactcccgagactctaatgctgatcttgtggtgttcggcggagggaccaaggtcaccgtcctaggtggcggcggaagcggcggaggctccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaa

SEQ ID NO: 30 - DNA, 37 bp, Artificial Sequence
  Description of Artificial Sequence Primer
gactgatatcgccaccatgagtgtgaagggcatggct

SEQ ID NO: 31 - DNA, 42 bp, Artificial Sequence
  Description of Artificial Sequence Primer
atcaaaaaagttgaaagaaagaattttgggggtggaggcagc

SEQ ID NO: 32 - DNA, 42 bp, Artificial Sequence
  Description of Artificial Sequence Primer
gctgcctccacccccaaaattctttctttcaacttttttgat

SEQ ID NO: 33 - DNA, 36 bp, Artificial Sequence
  Description of Artificial Sequence Primer
gggggtggaggcagccaggtacagctgcagcaatca

SEQ ID NO: 34 - DNA, 30 bp, Artificial Sequence
  Description of Artificial Sequence Primer
caaggtcaccgtcctaggttaagcggccgc

SEQ ID NO: 35 - DNA, 30 bp, Artificial Sequence
  Description of Artificial Sequence Primer
gcggccgcttaacctaggacggtgaccttg

SEQ ID NO: 36 - DNA, 1371 bp, Homo sapiens
ctccttccaagaagagcagcaaagctgaagtagcagcaacagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagggcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccagcaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtcaaaagcatctatgtgtcgtaaagcattcctcaaacattttttcatgcaaatacacayttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttgaaatacaaaatgtttttgtctaccaaagaaaaatgttgaaaaataagcaaatgtatacctagcaatcacttttactttttgtaattctgtctcttagaaaaatacataatctaatcaaaaaaaaaaaaaaaaaaaaaa

SEQ ID NO: 37 - protein, 5 aa, Artificial Sequence
  Description of Artificial Sequence Synthetic peptide
LRKED

SEQ ID NO: 38 - protein, 7 aa, Artificial Sequence
  Description of Artificial Sequence Synthetic peptide
QLFVNEE

SEQ ID NO: 39 - protein, 5 aa, Artificial Sequence
  Description of Artificial Sequence Synthetic peptide
LNQLT

SEQ ID NO: 40 - protein, 5 aa, Artificial Sequence
  Description of Artificial Sequence Synthetic peptide
YWCKW

SEQ ID NO: 41 - protein, 5 aa, Artificial Sequence
  Description of Artificial Sequence Synthetic peptide
GWYWC

SEQ ID NO: 42 - protein, 6 aa, Artificial Sequence
  Description of Artificial Sequence Synthetic peptide
STLVPL

SEQ ID NO: 43 - protein, 5 aa, Artificial Sequence
  Description of Artificial Sequence Synthetic peptide
SYRTD

SEQ ID NO: 44 - protein, 6 aa, Artificial Sequence
  Description of Artificial Sequence Synthetic peptide
QDPRLF

SEQ ID NO: 45 - protein, 5 aa, Artificial Sequence
  Description of Artificial Sequence Synthetic peptide
KRSSK

SEQ ID NO: 46 - protein, 5 aa, Artificial Sequence
  Description of Artificial Sequence Linker peptide
GGGGS

SEQ ID NO: 47 - protein, 4 aa, Artificial Sequence
  Description of Artificial Sequence Linker peptide
GSGS

SEQ ID NO: 48 - protein, 4 aa, Artificial Sequence
  Description of Artificial Sequence Linker peptide
GSSG

SEQ ID NO: 49 - protein, 6 aa, Artificial Sequence
  Description of Artificial Sequence 6-His tag
HHHHHH

SEQ ID NO: 50 - protein, 8 aa, Artificial Sequence
  Description of Artificial Sequence Linker peptide
GGGSGGGS

SEQ ID NO: 51 - protein, 6 aa, Artificial Sequence
  Description of Artificial Sequence Linker peptide
GGSGGS

SEQ ID NO: 52 - protein, 5 aa, Artificial Sequence
  Description of Artificial Sequence Linker peptide
GGGGC

SEQ ID NO: 53 - DNA, 774 bp, Artificial Sequence
  Description of Artificial Sequence Nucleotide sequence of pIgR-directed sFv (APLP10)
atgaaatacctattgcctacggcagccgctggattgttattactcgcggcccagccggccatggcccaggtacagctgcagcaatcagggggaggcgtggtccagcctgggaggtccctgagactctcctgtgcagcctctggattcaccttcagtagctatgctatgcactgggtccgccaggctccagggaaggggctggagtgggtctcagctattagtggtagtggtggtagcacatactacgcagactccgtgaagggccggttcaccatctccagagacaacgccaagaactcactgtatctgcaaatgaacagcctgagagccgaggacacggctgtgtattactgtgcgagagatacccgagggtacttcgatctctggggccgtggcaccctggtcaccgtctcctcaggtggcggagggtcatctgagctgactcaggaccctgctatgtctgtggccttgggacagacagtcagaatcacatgtcaaggggacagtctcagaaagtatcatgcaagctggtatcagcagaagccagggcaggcccctgttcttgtcatctatggtaagaatgaacgtccctcagggatcccagagcgattctctgggtccacctcaggagacacagcttccttgaccatcagtgggctccaggcggaagatgaggctgactattactgtcactcccgagactctaatgctgatcttgtggtgttcggcggagggaccaaggtcaccgtcctaggttaataagtcgacctcgac

SEQ ID NO: 54 - DNA, 1197 bp, Artificial Sequence
  Description of Artificial Sequence Nucleotide sequence of pIgR-directed sFv-IL-2 fusion construct
atgaaatacctattgcctacggcagccgctggattgttattactcgcggcccagccggccatggcccaggtacagctgcagcaatcagggggaggcgtggtccagcctgggaggtccctgagactctcctgtgcagcctctggattcaccttcagtagctatgctatgcactgggtccgccaggctccagggaaggggctggagtgggtctcagctattagtggtagtggtggtagcacatactacgcagactccgtgaagggccggttcaccatctccagagacaacgccaagaactcactgtatctgcaaatgaacagcctgagagccgaggacacggctgtgtattactgtgcgagagatacccgagggtacttcgatctctggggccgtggcaccctggtcaccgtctcctcaggtggcggagggtcatctgagctgactcaggaccctgctatgtctgtggccttgggacagacagtcagaatcacatgtcaaggggacagtctcagaaagtatcatgcaagctggtatcagcagaagccagggcaggcccctgttcttgtcatctatggtaagaatgaacgtccctcagggatcccagagcgattctctgggtccacctcaggagacacagcttccttgaccatcagtgggctccaggcggaagatgaggctgactattactgtcactcccgagactctaatgctgatcttgtggtgttcggcggagggaccaaggtcaccgtcctaggtggcggcggaagcggcggaggctccgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaacttaataactcgaggaattc